(19) United States
(12) Patent Application Publication
(10) Pub. No.: US 2020/0093165 A1
(43) Pub. Date: Mar. 26, 2020

(54) NATURALLY SWEET ENHANCER COMPOSITION

(71) Applicant: EPC Natural Products Co., Ltd, Beijing (CN)

(72) Inventors: Jingang Shi, Beijing (CN); Hansheng Wang, Beijing (CN); Thomas Eidenberger, Wels (AT)

(21) Appl. No.: 16/434,292

(22) Filed: Jun. 7, 2019

Related U.S. Application Data
(60) Provisional application No. 62/683,154, filed on Jun. 11, 2018.
(60) Provisional application No. 62/729,524, filed on Sep. 11, 2018.
(60) Provisional application No. 62/857,875, filed on Jun. 6, 2019.

(51) Int. Cl. A23L 27/30

(52) U.S. Cl. CPC A23L 27/36; A23V 2002/00

(57) ABSTRACT

The invention describes the use of one or more steviol glycosides (SGs) having a molecular weight (daltons) of less than or equal to about 965 to improve sweetness profiles of compositions, including sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof.

### CROSS REFERENCE TO RELATED APPLICATIONS

This application claims priority to U.S. Provisional Ser. No. 62/683,154, filed Jun. 11, 2018, U.S. Provisional Ser. No. 62/729,524, filed Sep. 11, 2018 and Provisional Ser. No. 62/857,875, filed Jun. 6, 2019, the contents of which are expressly incorporated herein by reference for all purposes.

### FIELD OF THE INVENTION

The invention relates generally to use of one or more steviol glycosides (SGs) having a molecular weight (daltons) of less than or equal to about 965 to improve sweetness profiles of compositions, including sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof.

### BACKGROUND OF THE INVENTION

Steviol glycosides and other natural sweeteners such as monk fruit extracts and sweet tea extracts are attractive alternative solutions for the reduction of sugar in consumable products. However, the taste profiles of each of these are still not satisfying to the consumer. Many researchers have been focusing on developing sources of large molecules of steviol glycosides, such as Reb D and Reb M, by enzymatic methods, fermentation methods or breeding of new varieties of stevia plants to obtain higher content of these two types of steviol glycosides. However, neither Reb D nor Reb M have been able to replace the sweetness associated with sugar at higher levels in composition due to their mouthfeel void and/or strong sweet lingering taste.

Therefore, there is need to develop a method and compositions to overcome one or more of the current disadvantages and to make steviol glycosides and other natural sweeteners suitable for the reduction of sugar in consumables.

### BRIEF SUMMARY OF THE INVENTION

Up until the present embodiments, little attention has been paid to low molecular weight steviol glycosides. In general, these compounds have been regarded as source of poor taste within the steviol glycoside family. Additionally, their content in stevia leaves is very low. The inventors have surprisingly found great value in these low molecular weight compounds for improving the taste profile of higher molecular weight steviol glycosides. Use of compositions that contain one or more of the lower molecular weight steviol glycosides (SG) having a molecular weight of less than or equal to 965 daltons, more particularly a molecular weight less than or equal to 787, provides a reduction in the lingering of sweetness typically associated with steviol glycoside compositions in comparison to sugar, with enhanced mouthfeel, and/or also providing a synergistic effect between sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof and overall sweetness of the resultant compositions/consumables.

The low molecular weight SGs useful for improving the taste profiles of the noted materials include SGs comprising one or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1.

In some embodiments, the sweetener compositions described herein comprise one or more SGs having a molecular weight less than or equal to 965 daltons, more particularly a molecular weight less than or equal to 787. In some embodiments, the sweetener composition comprises one or more SGs having a molecular weight less than or equal to 949 daltons. In some embodiments, the sweetener composition comprises one or more SGs having a molecular weight less than or equal to 935 daltons. In some embodiments, the sweetener composition comprises one or more SGs having a molecular weight less than or equal to 803 daltons. In some embodiments, the sweetener composition comprises one or more SGs having a molecular weight less than or equal to 787 daltons. In some embodiments, the sweetener composition comprises one or more SGs having a molecular weight less than or equal to 773 daltons. In some embodiments, the sweetener composition comprises one or more SGs having a molecular weight less than or equal to 675 daltons. In some embodiments, the sweetener composition comprises one or more SGs having a molecular weight less than or equal to 641 daltons. In some embodiments, the sweetener composition comprises one or more SGs having a molecular weight less than or equal to 625 daltons. In some embodiments, the sweetener composition comprises one or more SGs having a molecular weight less than or equal to 611 daltons. In some embodiments, the sweetener composition comprises one or more SGs having a molecular weight less than or equal to 479 daltons. In some embodiments, the sweetener composition comprises one or more SGs having a molecular weight less than or equal to 457 daltons.

The present invention surprisingly provides sweetener compositions, products, processes to prepare sweetener compositions described herein and uses of the sweetener compositions described herein comprising one or more steviol glycoside(s) (SGs) having a molecular weight (daltons) of less than or equal to about 965, more particularly a molecular weight less than or equal to 787, to improve sweetness, bitterness, off-taste, licorice after taste, after taste(s) and/or lingering of sweetness profiles of sweetener compositions, including sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 depicts the sweetness threshold of STM.

FIG. 2 depicts the sweetness threshold of STB.

FIG. 3 depicts the sweetness threshold of RU.

FIG. 4 depicts the sweetness threshold of DB.

FIG. 5 depicts an example for sweetness profile.

FIG. 6 depicts a sweetness profile for rebaudioside A (REB A) at concentrations of 3%, 5% and 7% in water.

FIG. 7 depicts a sweetness profile for rebaudioside B (REB B) at concentrations of 3%, 5% and 7% in water.

FIG. 8 depicts a sweetness profile for rebaudioside D (REB D) at concentrations of 3%, 5% and 7% in water.

FIG. 9 depicts a sweetness profile for rubusoside (RUB) at a concentration of 3% in water.

FIG. 10 depicts the sweetness profile for rebaudioside A (Reb-A) with rubusoside (Rub) at concentrations of 3%, 5% and 7% in water.

FIG. 11 depicts the sweetness profile for rebaudioside B (Reb-B) with rubusoside (Rub) at concentrations of 3%, 5% and 7% in water.

FIG. 12 depicts the sweetness profile for rebaudioside D (Reb-D) with rubusoside (Rub) at concentrations of 3%, 5% and 7% in water.

FIG. 13 depicts the sweetness profile of sucrose at concentration of 5% in water.

FIG. 14 depicts the sweetness-intensity/time profile of steviol-glycoside solutions.

FIG. 15 depicts the sweetness-intensity/time profile of 150 ppm Reb-A.

FIG. 16 depicts the sweetness-intensity/time profile of 150 ppm Reb-A plus 50 ppm Rubusoside

FIG. 17 depicts the sweetness-intensity/time profile of 150 ppm Reb-A plus 50 ppm steviolbioside.

FIG. 18 is an exemplary depiction of onset of sweetness (1), maximum sweetness (2), lingering sweetness (no decay of sweetness) (3), lingering off (decay of sweetness) (4) and no sweet taste remaining (5).

FIG. 19 denotes the sweetness/time profile for a 50 ppm solution of EPCalin 45%.

FIG. 20 denote the sweetness/time profile for a 50 ppm solution EPCalin 45% with 90 ppm rubusoside.

FIG. 21 denotes the sweetness/time profile for a 50 ppm solution of EPCalin 45% with 90 ppm steviolbioside.

FIG. 22 depicts the time/sweetness profile of 90% rubusoside (250 ppm solution).

FIG. 23 depicts the time/sweetness profile of 90% steviolbioside (250 ppm solution).

FIG. 24 depicts the sweetness profiles for different ratios of RD and STB.

FIG. 25 depicts the sweetness profiles for different ratios of DB and RD.

FIG. 26 depicts the sweetness profiles for different ratios of STM and RD.

FIG. 27 depicts the sweetness profiles for different ratios of RU and RD.

FIG. 28 depicts the sweetness profiles for different ratios of STB and RM.

FIG. 29 depicts the sweetness profiles for different ratios of DB and RM.

FIG. 30 depicts the sweetness profiles for different ratios of STM and RM.

FIG. 31 depicts the sweetness profiles for different ratios of RU and RM.

FIG. 32 depicts the sweetness profiles for different ratios of RA97 and RU.

FIG. 33 depicts the sweetness profiles for different ratios of RA97 and STB.

FIG. 34 depicts the sweetness profiles for different ratios of RA97 and DB.

FIG. 35 depicts the sweetness profiles for different ratios of RA97 and STM.

FIG. 36 depicts the sweetness profiles for different ratios of STB+RU(1/1) and RD+RM (9/1).

FIG. 37 depicts the sweetness profiles for different ratios of STB+STM(2/3) and RD+RM (5/5).

FIG. 38 depicts the sweetness profiles for different ratios of the hydrolyzate of RA50/SG95 to RM+RD(5/5).

FIG. 39 depicts the effects of added Reb-B on a standard NHDC (10 ppm) solution.

FIG. 40 depicts the effects of added rubusoside on a standard NHDC (10 ppm) solution.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The phrase “sucrose equivalence” or “SE” is the amount of non-sucrose sweetener required to provide the sweetness of a given percentage of sucrose in the same food, beverage, or solution. For instance, a non-diet soft drink typically contains 12 grams of sucrose per 100 ml of water, i.e., 12% sucrose. This means that to be commercially accepted, diet soft drinks must have the same sweetness as a 12% sucrose soft drink, i.e., a diet soft drink must have a 12% SE. Soft drink dispensing equipment assumes an SE of 12%, since such equipment is set up for use with sucrose-based syrups.

The phrase “sucrose equivalence” or “SE” is the amount of non-sucrose sweetener required to provide the sweetness of a given percentage of sucrose in the same food, beverage, or solution. For instance, a non-diet soft drink typically contains 12 grams of sucrose per 100 ml of water, i.e., 12% sucrose. This means that to be commercially accepted, diet soft drinks must have the same sweetness as a 12% sucrose soft drink, i.e., a diet soft drink must have a 12% SE. Soft drink dispensing equipment assumes an SE of 12%, since such equipment is set up for use with sucrose-based syrups. The phrase “taste profile” which is interchangeable with “sensory profile” or “aroma” and is defined as the temporal profile of all basic tastes of a sweetener. The onset and decay of sweetness when a sweetener is consumed, as perceived by trained human tasters and measured in seconds from first contact with a taster's tongue (“onset”) to a cutoff point (typically 180 seconds after onset), is called the “temporal profile of sweetness”. A plurality of such human tasters is called a “sensory panel”. In addition to sweetness, sensory panels can also judge the temporal profile of the other “basic tastes”: bitterness, saltiness, sourness, piquance (aka spiciness), and umami (aka savoriness or meatiness). The onset and decay of bitterness when a sweetener is consumed, as perceived by trained human tasters and measured in seconds from first perceived taste to the last perceived aftertaste at the cutoff point, is called the “temporal profile of bitterness”. Aroma from aroma producing substances are volatile compounds which are perceived by the odor receptor sites of the smell organ, i.e. the olfactory tissue of the nasal cavity. They reach the receptors when drawn in through the nose (orthonasal detection) and via the throat after being released by chewing (retronasal detection). The concept of aroma substances, like the concept of taste substances, should be used loosely, since a compound might contribute to the typical odor or taste of one food, while in another food it might cause a faulty odor or taste, or both, resulting in an off-flavor. Sensory profile includes evaluation of aroma as well.

The phrase “sweetness detection threshold” refers to the minimum concentration at which panelists consisting of 8 persons are able to detect sweetness in a composition, liquid or solid. This is further defined as provided in the Examples herein and are conducted by the methods described in Sensory Testing for Flavorings with Modifying Properties by Christie L. Harman, John B. Hallagan, and the FEMA Science, Committee Sensory Data Task Force, November 2013, Volume 67, No. 11 and Appendix A attached thereto the teachings of which are incorporated herein by reference.

“Threshold of sweetness” refers to a concentration of a material that below a concentration where sweetness can be detected may still impart a flavor to the consumable (including water). When half of a trained panel of testers determines something is “sweet” at a given concentration, then the sample meets the threshold. When less than half of a panel of testers cannot discern sweetness at a given concentration, then concentrations of the substance below the sweetness level are considered a flavoring.

The term “flavor” or “flavor characteristic”, as used herein, is the combined sensory perception of the components of taste, odor, and/or texture. The term “enhance”, as used herein, includes augmenting, intensifying, accentuating, magnifying, and potentiating the sensory perception of a flavor characteristic without changing the nature or quality thereof. The term “modify”, as used herein, includes altering, varying, suppressing, depressing, fortifying and supplementing the sensory perception of a flavor characteristic where the quality or duration of such characteristic was deficient.

The phrase “sweetener composition” as used herein refers to compositions that contain at least one, e.g., two, three, four, five, six or more low molecular weight SG(s) having a molecular weight of less than or equal to 965, more particularly a molecular weight less than or equal to 787, in combination with one or more of sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof and optional additives.

In one aspect, the sweetener compositions described herein advantageously provide a sugar like taste profile where there is a decreased, eliminated or masked aftertaste (of for example a metallic taste or licorice taste) or a decreased, eliminated or masked bitterness or decreased, eliminated or masked lingering of sugar taste in comparison to stevia, sweet tea or mogroside based products without the inclusion of the low molecular weight SG(s) at a level sufficient to decrease, eliminate, or mask the undesired taste profile.

As used herein, the term “rebaudioside” can be abbreviated as “Reb” or “R.” For example, the phrase “Rebaudioside A” has the same meaning as “Reb A” or “RA.” The same holds true for all rebaudiosides.

The term steviol glycoside (“SG”) is recognized in the art and is intended to include the major and minor constituents of stevia as listed, for example, in Table A. These include, but are not limited to components of stevia such as steviol, STB, ST, RA, RB, RC, RD, RE, RF, RM (also referred to as Rebaudioside X (RX)), SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1. Herein, Dulcoside A has the same definition as Dulcoside. The SG can be purified before use.

Non-limiting examples of steviol glycosides are shown in Table A below. The stevia glycosides for use in the present application are not limited by source or origin. Steviol glycosides may be extracted from stevia leaves, synthesized by enzymatic processes, synthesized by chemical syntheses, or produced by fermentation.

As used herein, the terms “steviol glycoside,” (“SG”) refers to a glycoside of steviol, a diterpene compound shown in Formula I.

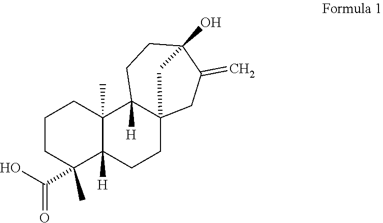

As shown in Formula II, SGs are a steviol molecule glycosylated with a parent or core structure comprising a steviol molecule with glycosylation at the C13 and/or C19 position.

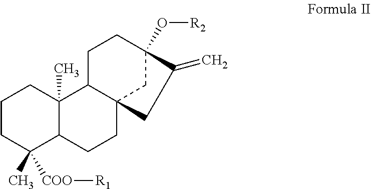

As shown in Formula III, steviol glycosides can also have a parent or core structure comprising an iso-steviol (Iso-Sv) molecule with glycosylation at the C13.

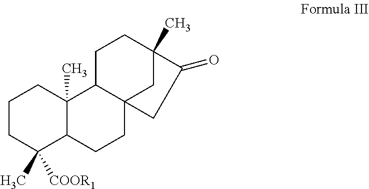

In some embodiments of SGs having a parent or core structure of Formula II or Formula III, R1 and R2 are substituent groups individually selected from the groups comprising glucosyl (G), rhamnosyl (R), xylosyl (X), deoxy-glucosyl (dG), frucosyl (F), arabinosyl (A) or galactosyl (Ga) group. In some further embodiments, the number of the glucosyl group is equal to or greater than 4.

Table A provides a list of about 80 SGs for use in the present application.

TABLE A

| | | | Rhamnose | Xylose | | | |
|---|---|---|---|---|---|---|---|
| | | | (mr = 164) | (mr = 150) | | | |
| | | Glucose | Deoxyhexose | Arabinose | R1 | R2 | |
| Name | [M − H]− | (mr = 180) | (mr = 164) | (mr = 150) | (C-19) | (C-13) | Backbone |
| Related SvGn#1 | 457 | | | | | | |
| Steviol-monoside | 479 | 1 | | | H— | Glcβ1- | Steviol |
| Steviol-monoside | 479 | 1 | 1 | | Glcβ1- | H— | |
| A | | | | | | | |
| SG-4 | 611 | 1 | | 1 | H— | Xylβ(1-2)Glcβ1- | Steviol |
| Dulcoside A1 | 625 | 1 | 1 | | H— | Rhaα(1-2)Glcβ1- | Steviol |
| Iso-Steviolbioside | 641 | 2 | | | H— | Glcβ(1-2)Glcβ1- | Isosteviol |
| Reb-G1 | 641 | 2 | | | H— | Glcβ(1-3)Glcβ1- | Steviol |
| rubusoside | 641 | 2 | | | Glcβ1- | Glcβ1- | Steviol |
| steviolbioside | 641 | 2 | | | H— | Glcβ(1-2)Glcβ1- | Steviol |
| Related SvGn#3 | 675 | | | | | | |
| Reb-F1 | 773 | 2 | | 1 | H— | Xylβ(1-2)[Glcβ(1- | Steviol |
| | | | | | | 3)]Glcβ1- | |
| Reb-R1 | 773 | 2 | | 1 | H— | Glcβ(1-2)[Glcβ(1- | Steviol |
| | | | | | | 3)]Xylβ1- | |
| Stevioside F | 773 | 2 | | 1 | Glcβ1- | Xylβ(1-2)Glcβ1- | Steviol |
| (SG-1) | | | | | | | |
| SG-Unk1 | 773 | 2 | | 1 | — | — | Steviol |
| dulcoside A | 787 | 2 | 1 | | Glcβ1- | Rhaα(1-2)Glcβ1- | Steviol |
| dulcoside B | 787 | 2 | 1 | | H— | Rhaα(1-2)[Glcβ(1- | Steviol |
| (JECFA C) | | | | | | 3)]Glcβ1- | |
| SG-3 | 787 | 2 | 1 | | H— | 6-deoxyGlcβ(1- | Steviol |
| | | | | | | 2)[Glcβ(1-3)]Glcβ1- | |
| Stevioside D | 787 | 2 | 1 | | Glcβ1- | Glcβ(1-2)6-deoxyGlcβ1- | |
| Iso-Reb B | 803 | 3 | | | H— | Glcβ(1-2)[Glcβ(1- | Isosteviol |
| | | | | | | 3)]Glcβ1- | |
| Iso-Stevioside | 803 | 3 | | | Glcβ1- | Glcβ(1-2)Glcβ1- | Isosteviol |
| Reb B | 803 | 3 | | | H— | Glcβ(1-2)[Glcβ(1- | Steviol |
| | | | | | | 3)]Glcβ1- | |
| Reb G | 803 | 3 | | | Glcβ1- | Glcβ(1-3)Glcβ1- | Steviol |
| Reb-KA | 803 | 3 | | | Glcβ(1- | Glcβ1- | Steviol |
| | | | | | 2)Glcβ1- | | |
| SG-13 | 803 | 3 | | | Glcβ1- | Glcβ(1-2)Glcβ1- | Isomeric |
| | | | | | | | steviol |
| | | | | | | | (12a- |
| | | | | | | | hydroxy) |
| Stevioside | 803 | 3 | | | Glcβ1- | Glcβ(1-2)Glcβ1- | Steviol |
| Stevioside B | 803 | 3 | | | Glcβ(1- | Glcβ1- | Steviol |
| (SG-15) | | | | | 3)Glcβ1- | | |
| Reb F | 935 | 3 | | 1 | Glcβ1- | Xylβ(1-2)[Glcβ(1- | Steviol |
| | | | | | | 3)]Glcβ1- | |
| Reb R | 935 | 3 | | 1 | Glcβ1- | Glcβ(1-2)[Glcβ(1- | Steviol |
| | | | | | | 3)]Xylβ1- | |
| SG-Unk2 | 935 | 3 | | 1 | — | — | Steviol |
| SG-Unk3 | 935 | 3 | | 1 | — | — | Steviol |
| REb F3 (SG-11) | 935 | 3 | | 1 | Xylβ(1- | Glcβ(1-2)Glcβ1- | Steviol |
| | | | | | 6)Glcβ1- | | |
| Reb F2 (SG-14) | 935 | 3 | | 1 | Glcβ1- | Glcβ(1-2)[Xylβ(1- | Steviol |
| | | | | | | 3)]Glcβ1- | |
| Reb C | 949 | 3 | 1 | | Glcβ1- | Rhaα(1-2)[Glcβ(1- | Steviol |
| | | | | | | 3)]Glcβ1- | |
| Reb C2/Reb S | 949 | 3 | 1 | | Rhaα(1- | Glcβ(1-2)Glcβ1- | Steviol |
| | | | | | 2)Glcβ1- | | |
| Stevioside E | 949 | 3 | 1 | | Glcβ1- | 6-DeoxyGlcβ(1- | Steviol |
| (SG-9) | | | | | | 2)[Glcβ(1-3)]Glcβ1- | |
| Stevioside E2 | 949 | 3 | 1 | | 6- | Glcβ(1-2)[Glcβ(1- | |
| | | | | | DeoxyGlcβ1- | 3)]Glcβ1- | |
| SG-10 | 949 | 3 | 1 | | Glcβ1- | Glcα(1-3)Glcβ(1- | Steviol |
| | | | | | | 2)[Glcβ(1-3])Glcβ1- | |
| Reb L1 | 949 | 3 | 1 | | H— | Glcβ(1-3)Rhaα(1- | Steviol |
| | | | | | | 2)[Glcβ(1-3)]Glcβ1- | |
| SG-2 | 949 | 3 | 1 | | Glcβ1- | 6-deoxyGlcβ(1- | Steviol |
| | | | | | | 2)[Glcβ(1-3)]Glcβ1- | |
| Reb A3 (SG-8) | 965 | 4 (1 Fru) | | | Glcβ1- | Glcβ(1-2)[Fruβ(1- | |
| | | | | | | 3)]Glcβ1- | |
| Iso-Reb A | 965 | 4 | | | Glcβ1- | Glcβ(1-2)[Glcβ(1- | Isosteviol |
| | | | | | | 3)]Glcβ1- | |
| Reb A | 965 | 4 | | | Glcβ1- | Glcβ(1-2)[Glcβ(1- | Steviol |
| | | | | | | 3)]Glcβ1- | |
| Reb A2 (SG-7) | 965 | 4 | | | Glcβ1- | Glcβ(1-6)[Glcβ(1- | Steviol |
| | | | | | | 2)]Glcβ1- | |
| Reb E | 965 | 4 | | | Glcβ(1- | Glcβ(1-2)Glcβ1- | Steviol |
| | | | | | 2)Glcβ1- | | |
| Reb H1 | 965 | 4 | | | H— | Glcβ(1-6)Glcβ(1- | Steviol |
| | | | | | | 3)[Glcβ1-3)]Glcβ1- | |
| Related SvGn#2 | 981 | | | | | | |
| Related SvGn#5 | 981 | | | | | | |
| Reb U2 | 1097 | 4 | | 1 | Xylβ(1- | Glcβ(1-2)Glcβ1- | |
| | | | | | 2)[Glcβ(1- | | |
| | | | | | 3)Glcβ1- | | |
| Reb T | 1097 | 4 | | 1 | Xylβ(1- | Glcβ(1-2)[Glcβ(1- | |
| | | | | | 2)Glcβ1- | 3)]Glcβ1- | |
| Reb W | 1097 | 4 | | 1 | Glcβ(1- | Glcβ(1-2)Glcβ1- | |
| | | | | | 2)[Araβ(1- | | |
| | | | | | 3)]Glcβ1- | | |
| Reb W2 | 1097 | 4 | | 1 | Araβ(1- | Glcβ(1-2)[Glcβ(1- | |
| | | | | | 2)Glcβ1- | 3)]Glcβ1- | |
| Reb W3 | 1097 | 4 | | 1 | Araβ(1- | Glcβ(1-2)[Glcβ(1- | |
| | | | | | 6)Glcβ1- | 3)]Glcβ1- | |
| Reb U | 1097 | 4 | | 1 | Araα(1-2)- | Glcβ(1-2)[Glcβ(1- | Steviol |
| | | | | | Glcβ1- | 3)]Glcβ1- | |
| SG-12 | 1111 | 4 | 1 | | Rhaα(1- | Glcβ(1-2)[Glcβ(1- | Steviol |
| | | | | | 2)Glcβ1- | 3)]Glcβ1- | |
| Reb H | 1111 | 4 | 1 | | Glcβ1- | Glcβ(1-3)Rhaα(1- | Steviol |
| | | | | | | 2)[Glcβ(1-3)]Glcβ1- | |
| Reb J | 1111 | 4 | 1 | | Rhaα(1- | Glcβ(1-2)[Glcβ(1- | Steviol |
| | | | | | 2)Glcβ1- | 3)]Glcβ1- | |
| Reb K | 1111 | 4 | 1 | | Glcβ(1- | Rhaα(1-2)[Glcβ(1- | Steviol |
| | | | | | 2)Glcβ1- | 3)]Glcβ1- | |
| Reb K2 | 1111 | 4 | 1 | | Glcβ(1- | Rhaα(1-2)[Glcβ(1- | Steviol |
| | | | | | 6)Glcβ1- | 3)]Glcβ1- | |
| SG-Unk4 | 1111 | 4 | 1 | | — | — | Steviol |
| SG-Unk5 | 1111 | 4 | 1 | | — | — | Steviol |
| Reb D | 1127 | 5 | | | Glcβ(1- | Glcβ(1-2)[Glcβ(1- | Steviol |
| | | | | | 2)Glcβ1- | 3)]Glcβ1- | |
| Reb I | 1127 | 5 | | | Glcβ(1- | Glcβ(1-2)[Glcβ(1- | Steviol |
| | | | | | 3)Glcβ1- | 3)]Glcβ1- | |
| Reb L | 1127 | 5 | | | Glcβ1- | Glcβ(1-6)Glcβ(1- | Steviol |
| | | | | | | 2)[Glcβ(1-3)]Glcβ1- | |
| Reb I3 | 1127 | 5 | | | [Glcβ(1- | Glcβ(1-2)Glcβ1- | |
| | | | | | 2)Glcβ | | |
| | | | | | (1-6)]Glcβ1- | | |
| SG-Unk6 | 1127 | 5 | | | — | — | Steviol |
| Reb Q (SG-5) | 1127 | 5 | | | Glcβ1- | Glcα(1-4)Glcβ(1- | Steviol |
| | | | | | | 2)[Glcβ(1-3)]Glcβ1- | |
| Reb I2 (SG-6) | 1127 | 5 | | | Glcβ1- | Glcα(1-3)Glcβ1- | Steviol |
| | | | | | | 2[Glcβ1-3)]Glcβ1- | |
| Reb Q2 | 1127 | 5 | | | Glcα(1- | Glcβ(1-2)Glcβ1- | |
| | | | | | 2)Glcα(1- | | |
| | | | | | 4)Glcβ1- | | |
| Reb Q3 | 1127 | 5 | | | Glcβ1- | Glcα(1-4)Glcβ(1- | |
| | | | | | | 3)[Glcβ(1-2)]Glcβ1- | |
| Reb T1 | 1127 | 5 (1 Gal) | | | Galβ(1- | Glcβ(1-2)[Glcβ(1- | |
| | | | | | 2)Glcβ1- | 3)]Glcβ1- | |
| Related SvGn#4 | 1127 | | | | | | |
| Reb V2 | 1259 | 5 | | 1 | Xylβ(1- | Glcβ(1-2)[Glcβ(1- | Steviol |
| | | | | | 2)[Glcβ(1- | 3)]Glcβ1- | |
| | | | | | 3)]- | | |
| | | | | | Glcβ1- | | |
| Reb V | 1259 | 5 | | 1 | Glcβ(1- | Xylβ(1-2)[Glcβ(1-3)]- | |
| | | | | | 2)[Glcβ(1- | Glcβ1- | |
| | | | | | 3)]Glcβ1- | | |
| Reb Y | 1259 | 5 | | 1 | Glcβ(1- | Glcβ(1-2)[Glcβ(1- | |
| | | | | | 2)[Araβ(1- | 3)]Glcβ1- | |
| | | | | | 3)]Glcβ1- | | |
| Reb N | 1273 | 5 | 1 | | Rhaα(1- | Glcβ(1-2)[Glcβ(1- | Steviol |
| | | | | | 2)[Glcβ(1- | 3)]Glcβ1- | |
| | | | | | 3)]Glcβ1- | | |
| Reb M | 1289 | 6 | | | Glcβ(1-2)[Glcβ(1- | Glcβ(1-2)[Glcβ(1- | Steviol |
| | | | | | 3)]Glcβ1- | 3)]Glcβ1- | |
| 15α-OH Reb M | 1305 | 6 | | | Glcβ1- | Glcβ1-2(Glcβ1-3)Glcβ1- | 15α- |
| | | | | | 2(Glcβ1- | | Hydroxysteviol |
| | | | | | 3)Glcβ1- | | |
| Reb O | 1435 | 6 | 1 | | Glcβ(1- | Glcβ(1-2)[Glcβ(1- | Steviol |
| | | | | | 3)Rhaα(1- | 3)]Glcβ1- | |
| | | | | | 2)[Glcβ(1- | | |
| | | | | | 3)]Glcβ1- | | |
| Reb O2 | 1435 | 6 | 1 | | Glcβ(1- | Glcβ(1-2)[Glcβ(1- | |
| | | | | | 4)Rhaα(1- | 3)]Glcβ1- | |
| | | | | | 2)[Glcβ(1- | | |
| | | | | | 3)]Glcβ1- | | |

“mr” refers to molecular mass
Legend: SG-1 to 16: SGs without a specific name; SG-Unk1-6: SGs without detailed structural proof; Glc: Glucose; Rha: Rhamnose; Xyl: Xylose; Ara: Arabinose.

As used herein, the term “glycosylated steviol glycoside” (“GSG”) refers to an SG with additional glucose residues added relative to the parental SGs present in e.g., Stevia leaves. Preferably, the GSGs are produced by an in vitro enzymatically catalyzed glycosylation process. A GSG may also be produced by chemical synthesis.

The phrase “glycosylated steviol glycoside(s)” (“GSG”, “GSGs”) as referred to herein, pertains to a steviol glycoside that is glycosylated at multiple positions (including partially glycosylated steviol glycosides) obtained, for example, by synthetic manipulation or by enzymatic processes, such as GSG-RA50. It should be understood that GSG(s) essentially contains a glycosylated steviol glycoside(s), but also contains unreacted steviol glycosides, dextrin and other non-steviol glycoside substances found in extracts. It should also be understood that the GSG(s) can be purified and/or separated into purified/isolated components.

The term “glycosylated steviol glycosides” (“GSGs”) refers to compounds obtained by enzymatic processes, for example, by transglycosylating stevia extract containing steviol glycosides, or by common known synthetic manipulation. Herein, the GSGs comprise glycosylated stevia extract containing glycosylated steviol glycoside(s) and also comprises short chain compounds obtained by hydrolyzation of glycosylated product, as well as non-glycosylated components which are the residue of unreacted steviol glycosides, or unreacted components other than steviol glycosides contained in the stevia extract. The methods and GSGs found in KR10-2008-0085811 are herein incorporated by reference. It should be understood that these GSGs can be purified and/or separated into purified/isolated components.

As used herein, the phrase “glycosylated steviol glycoside composition” or “GSG composition” refers to any material comprising one or more GSGs.

As used herein, the term “SG/GSG composition” refers to a generic composition that may comprise one or more SGs and/or one or more GSGs.

The phrase “total glycosides” refers to the total amount of GSGs and SGs in a composition.

In certain embodiments, the GSGs used in the present application are prepared as follows: i) dissolving a glucose-donor material in water to form a liquefied glucose-donor material; ii) adding a starting SG composition to liquefied glucose-donor material to obtain a mixture; iii) adding an effective amount of an enzyme to the mixture to form a reaction mixture, wherein the enzyme catalyzes the transfer of glucose moieties from the glucose-donor material to SGs in the starting SG composition, and incubating the reaction mixture at a desired temperature for a desired length of reaction time to glycosylate SGs with glucose moieties present in the glucose-donor molecule. In some further embodiments, after achieving a desired ratio of GSG- and residual SG contents, the reaction mixture can be heated to a sufficient temperature for a sufficient amount of time to inactivate the enzyme. In some embodiments, the enzyme is removed by filtration in lieu of inactivation. In other embodiments, the enzyme is removed by filtration following inactivation. In some embodiments the resulting solution comprising GSG, residual SGs and dextrin is decolorized. In certain embodiments the resulting solution of GSG, residual SGs and dextrin is dried. In some embodiments, the drying is by spray drying. In some embodiments, step (i) comprises the substeps of (a) mixing a glucose-donor material with a desired amount of water to form a suspension, (b) adding a desired amount of enzyme to the suspension and (c) incubate the suspension at a desired temperature for a desired time to form liquefied glucose-donor material. Starch can be a suitable substitute for dextrin(s) and/or dextrin(s) can be obtained by the hydrolysis of starch.

The term “mogroside” (“MG”) is a triterpene-glycoside and is recognized in the art and is intended to include the major and minor constituents of mogroside extracts.

Extracts from the fruits of Siraitia grosvenorii (Swingle), also known as Momordica grosvenori (Swingle), Luo Han Guo or monk fruit etc. provide a family of triterpene-glycosides and are referred to as mogroside(s) (“MGs”) throughout the specification. The extracts include, for example, mogroside V, mogroside IV, siamenoside I, and 11-oxomogroside V. Constituents of the mogroside extracts are referred to by the designation “MG” followed by symbol, such as “V”, therefore mogroside V is “MGV”. Siamenoside I would be “SSI”, 11-oxomogroside V would be “OGV”.

It should be understood that monk fruit extracts can contain, for example, a mogroside such as MGV, in an amount of 3% by weight, 5% by weight, 20% by weight, 40% by weight, 50% by weight, 60% by weight or higher but containing other mogrosides or non-mogrosides in the extracts. For example, other components include other mogrosides such as mogroside II, mogroside IIIA, mogroside IIIE, mogro side IVA, mogroside IVE, siamenoside I, and 11-oxomogroside V. In addition, some other polysaccharides or flavonoids may be present.

The mogroside(s) of interest can be purified before use.

The term “glycosylated mogroside(s)” (“GMG”, “GMGs”), refers to a mogroside that is glycosylated at least at one or more positions in addition to those positions glycosylated in native form, obtained, for example, by synthetic manipulation or by enzymatic processes.

The terms “glycosylated mogroside”, or “glycosylated swingle extract containing a glycosylated mogroside” refers to compounds obtained by transglycosylating swingle extract containing mogrosides, or transglycosylating purified mogrosides so as to add glucose units, for example, one, two, three, four, five or more than five glucose units, to the native mogrosides by glycosyltransferase, preferably, CGTase enzyme (cyclodextringlycosyltransferase). Herein, the glycosylated mogroside(s), or the glycosylated swingle extract containing glycosylated mogroside(s), comprises short chain compounds obtained by hydrolyzation of glycosylated product and also comprises non-glycosylated ingredients which are the residue of non-reacted mogrosides, or unreacted components other than mogrosides contained in the swingle extract.

A suitable procedure to prepare glycosylated mogrosides (GMGs) or glycosylated swingle extract(s) includes i) dissolving dextrin in water (e.g., reverse osmosis), ii) adding the mogrosides or extract to the solubilized dextrin to obtain a mixture, wherein the ratio of dextrin to mogrosides/extract is optimized in a ratio of between 100:1 to 1:100 with suitable ranges including 3:1, 2:1, 1.5:1 and 1:1, iii) adding CGTase enzyme to the mixture followed by incubating the mixture at 60° C. for a desired length of reaction time to glycosylate mogrosides with glucose molecules derived from dextrin.

After achieving the desired ratio of GMG(s) and residual mogroside(s) contents, the reaction mixture is heated to 90-100° C. for 30 minutes to inactivate the CGTase, which is then removed by filtration.

Optionally, amylase can be added to the mixture and the mixture is incubated at 70° C. for a desired length of reaction time to shorten the length of glucose chain(s) in the GMG molecules.

Decolorization and/or spray drying the resulting mixture of GMG, residual mogrosides and dextrin can then be undertaken.

It should be understood that GMG(s) essentially contains glycosylated mogroside(s), but also contains unreacted mogrosides, dextrin and other non-mogroside substances found in extracts. It should also be understood that the GMG(s) can be purified and/or separated into purified/isolated components.

A “glycosylated sweet tea extract” refers to a sweet tea extract that is glycosylated at least at one or more positions in addition to those positions glycosylated in native form, obtained, for example, by synthetic manipulation or by enzymatic processes.

The terms “glycosylated sweet tea glycosylate”, or “glycosylated sweet tea extract containing a glycosylated rubusoside or kaurane-type diterpene glycosides suaviosides B, G, H, I and J” refers to compounds obtained by transglycosylating sweet tea extract containing rubusoside or suaviosides, or transglycosylating purified sweet tea extract so as to add glucose units, for example, one, two, three, four, five or more than five glucose units, to the native rubusoside or suavioside(s) by glycosyltransferase, preferably, CGTase enzyme (cyclodextringlycosyltransferase). Herein, the glycosylated sweet tea glycosylates, comprises short chain compounds obtained by hydrolyzation of glycosylated product and also comprises non-glycosylated ingredients which are the residue of non-reacted rubusoside or suavioside(s), or unreacted components other than rubusoside or suavioside(s) contained in the sweet tea extract.

Not to be limited by the following, common methods of manufacturing of the sweetening agents (e.g., stevia extract) are as follows. The method presented should not be considered limiting.

Extract stevia leaves with water at 20-80° C. with the ratio of leaves to water being about 1:10 to 1:20 (w/v). The mixture can be clarified by flocculation or membrane filtration. The mixture can then be purified through a macroporous resin and ion exchange resin. The filtrate is then crystallized with a mixture of water/alcohol (ethanol or methanol) to obtain a precipitate which is then filtered and dried.

A swingle extract or mogroside extract containing mogrosides can be produced by the method of extracting the fruit of Siraitia grosvenorii (Swingle) with an alcohol, a mixture of alcohol and water, or water to obtain mixtures of mogrosides, then purified to provide desired mogrosides such as mogroside V. Specifically, a swingle extract containing mogrosides is produced by the method as follows: extraction of the fruit of Siraitia grosvenorii (Swingle) with an alcohol, a mixture of alcohol and water, or water to obtain the mogrosides (such as mogroside V etc.) component ranging from about 1% to 99% by weight of the extract. In a preferred embodiment, the swingle extract contains about 10-90% by weight mogrosides. In another preferred embodiment, the swingle extract contains about 20-80% by weight mogrosides. In another preferred embodiment, the swingle extract contains about 30-70% by weight mogrosides. In another preferred embodiment, the swingle extract contains about 40-60% by weight mogrosides.

A suitable process to obtain a mogroside extract (swingle extract) is provided as follows. Luo Han Guo fruit is extracted with water or a mixture of water/alcohol (ethanol or methanol) at a temperature of from about 40° C. to about 80° C. with the ratio of fruit to solvent being about 1:10 to about 1:20 (weight to volume). The liquid can be clarified by flocculation or membrane filtration followed by purification through a macroporous resin and ion exchange resin. Decolorization can be accomplished with activated carbon. Solids are then filtered and dried.

In one aspect, as an example, glycosylated mogroside V (GMGV), is produced by dissolving dextrin in water (reverse osmosis water). The ratio of GMGV to water is about 1:10 (weight/volume, (w/v)). A swingle extract with a mogroside content of between 1% and 99% is added to dextrin solution. In one embodiment, the dextrin to swingle extract ratio was optimized to a ratio of between 30:70 and 70:30. CGTase enzyme is added to the mixture (ratio of GMGV to CGTase is about 20:1 (w/v) and incubated at 60-70° C. for a desired length of reaction time (typically from about 2 hours to about 72 hours, more preferably from about 8 hours to about 48 hours, even more preferably from about 12 hours to about 24 hours) to glycosylate mogrosides with glucose molecules derived from dextrin, wherein the addition amount by volume is about 0.1-0.5 ml based on 1 g mogrosides. (The ratio of GMGV to CGTase is from about 10:1 to about 20:1 w/v). After the desired ratio of GMGs and residual mogroside and dextrin contents are achieved (monitored by HPLC to analyze the content of unreacted MGV), the reaction mixture is heated to 80-100° C. for 30 min to inactivate the CGTase, which is then removed by filtration. The resulting solution of GMGs, residual mogroside and dextrin is decolored and spray dried.

Low molecular weight steviol glycosides (“LMWSGs”) can be prepared, for example, by the hydrolysis of a given steviol glycoside. For example, treatment of stevioside with sodium hydroxide provides steviolbioside (STB) or a mixture of STB and stevioside with or without purification for use, such as a dried powder. If the hydrolyzed material is not purified, then the mixture contains stevioside, glucose, STB sodium salt and possible caramelized substances. Neutralization of the unpurified material with an acid provides a mixture that includes stevioside, glucose, STB, salt (e.g., sodium chloride, sodium sulfate, etc. depending on the acid utilized) and possible caramelized substances. The acidified products can be further purified by known purifications methods (recrystallization, column chromatography, HPLC, etc.) to provide pure STB or a mixture of STB and stevioside.

Another example is the hydrolysis of rubusoside to steviol monoside (STM). As noted above, the hydrolysis of the rubusoside produces STM or a mixture of STM and rubusoside and can be used with or without further purification. If the hydrolyzed materials is not purified, then the mixture contains rubusoside, glucose, STM and possible caramelized substances. Neutralization of the unpurified material with an acid provides a mixture that includes STM, rubusoside, glucose, salt and possible caramelized substances. The acidified products can be further purified as described above. All these materials can be used for improving the taste profile of the current embodiments.

It should be understood that throughout this specification, when reference is made to a specific sweetening agent, such as an SG, a GSG, an MG, or a GMG and the like, that the example is meant to be inclusive and applicable to sweet tea extracts, stevia extracts, swingle extracts (mogroside extract), single components or mixtures of mogroside(s) (“MGs”), steviol glycosides (“SGs”), sweet tea glycosides, glycosylated mogrosides (“GMGs”), glycosylated steviol glycosides (“GSGs”) and glycosylated sweet tea glycosides.

An acronym of the type “YYxx” refers to a composition, where YY refers to a compound (such as RA) or collection of compounds (e.g., SGs), where “xx” is typically a percent by weight number between 1 and 100 denoting the level of purity of a given compound (such as RA) or collection of compounds, where the weight percentage of YY in the dried product is equal to or greater than xx. Without specific description, the acronym “RAx” refers to a stevia composition containing RA in amount of ≥x % and <(x+10)% with the following exceptions: The acronym “RA100” specifically refers to pure RA; the acronym “RA99.5” specifically refers to a composition where the amount of RA is ≥99.5 wt %, but <100 wt %; the acronym “RA99” specifically refers to a composition where the amount of RA is ≥99 wt %, but <100 wt %; the acronym “RA98” specifically refers to a composition where the amount of RA is ≥98 wt %, but <99 wt %; the acronym “RA97” specifically refers to a composition where the amount of RA is ≥97 wt %, but <98 wt %; the acronym “RA95” specifically refers to a composition where the amount of RA is ≥95 wt %, but <97 wt %; the acronym “RA85” specifically refers to a composition where the amount of RA is ≥85 wt %, but <90 wt %; the acronym “RA75” specifically refers to a composition where the amount of RA is ≥75 wt %, but <80 wt %; the acronym “RA65” specifically refers to a composition where the amount of RA is ≥65 wt %, but <70 wt %; the acronym; the acronym “RA20” specifically refers to a composition where the amount of RA is ≥15 wt %, but <30 wt %.

The acronym “GSG-RAxx” refers to a GSG composition prepared in an enzymatically catalyzed glycosylation process with RAxx as the starting SG material. More generally, acronyms of the type “GSG-YYxx” refer to a composition of the present application where YY refers to a compound (such as RA, RB, RC or RD), or a composition (e.g., RA20), or a mixture of compositions (e.g., RA40+RB8). For example, GSG-RA20 refers to the glycosylation products formed from RA20.

The abbreviation “GX” is noted throughout the specification and refers to glycosyl groups “G” where “X” is a value from 1 to 20 and refers to the number of glycosyl groups present in the molecule. For example, Stevioside G1 (ST-G1) has one (1) glycosyl group (G), thus “G1”, Stevioside G2 (ST-G2) has two (2) glycosyl groups present, Stevioside G3 (ST-G3) has three (3) glycosyl groups present, Stevioside G4 (ST-G4) has four (4) glycosyl groups present, Stevioside G5 (ST-G5) has five (5) glycosyl groups present, Stevioside G6 (ST-G6) has six (6) glycosyl groups present, Stevioside G7 (ST-G7) has seven (7) groups present, Stevioside G8 (ST-G8) has eight (8) glycosyl groups present, Stevioside G9 (ST-G9) has nine (9) glycosyl groups present, etc. The glycosylation of the molecule can be determined by HPLC-MS.

Table B provides various GSG groups that are included herein. Table A depicts GSG groups corresponding to parental SGs with glucose (“G”; i.e., 2nd G after hyphen) moieties added thereto. For example, GSG-1G-2 refers to having one glucose added, and “2” is the series number in the row of Table B.

TABLE B

| | | | GSG group based on parental SG-group given | | | | | |
|---|---|---|---|---|---|---|---|---|
| Parental SG | | | mw = 480 | mw = 642 | mw = 804 | mw = 966 | mw = 1128 | mw = 1290 |
| Steviol-glycoside (GS) | SG-group | mw | SG-1G | SG-2G | SG-3G | SG-4G | SG-5G | SG-6G |
| Steviolmonoside | SG-1G | 480 | | | | | | |
| Steviolmonoside A | | | | | | | | |
| Iso-steviolbioside | SG-2G | 642 | GSG-1G-1 | | | | | |
| Reb-G1 | | | | | | | | |
| Rubusoside | | | | | | | | |
| Steviolbioside | | | | | | | | |
| Iso-Reb B | SG-3G | 804 | GSG-1G-2 | GSG-2G-1 | | | | |
| Iso-Stevioside | | | | | | | | |
| Reb B | | | | | | | | |
| Reb G | | | | | | | | |
| Reb-KA | | | | | | | | |
| SG-13 | | | | | | | | |
| Stevioside | | | | | | | | |
| Stevioside B (SG-15) | | | | | | | | |
| Reb A3 (SG-8) | SG-4G | 966 | GSG-1G-3 | GSG-2G-2 | GSG-3G-1 | | | |
| Iso-Reb A | | | | | | | | |
| Reb A | | | | | | | | |
| Reb A2 (SG-7) | | | | | | | | |
| Reb E | | | | | | | | |
| Reb H1 | | | | | | | | |
| Reb D | SG-5G | 1128 | GSG-1G-4 | GSG-2G-3 | GSG-3G-2 | GSG-4G-1 | | |
| Reb I | | | | | | | | |
| Reb L | | | | | | | | |
| Reb I3 | | | | | | | | |
| SG-Unk6 | | | | | | | | |
| Reb Q (SG-5) | | | | | | | | |
| Reb 12 (SG-6) | | | | | | | | |
| Reb Q2 | | | | | | | | |
| Reb Q3 | | | | | | | | |
| Reb T1 | | | | | | | | |
| Related SvGn#4 | | | | | | | | |
| Reb M | SG-6G | 1290 | GSG-1G-5 | GSG-2G4 | GSG-3G-3 | GSG-4G-2 | GSG-5G-1 | |
| — | — | 1452 | GSG-1G-6 | GSG-2G-5 | GSG-3G-4 | GSG-4G-3 | GSG-5G-2 | GSG-6G-1 |
| — | — | 1614 | GSG-1G-7 | GSG-2G-6 | CSG-3G-5 | GSG-4G-4 | GSG-5G-3 | GSG-6G-2 |
| — | — | 1776 | GSG-1G-8 | GSG-2G-7 | GSG-3G-6 | GSG-4G-5 | GSG-5G-4 | GSG-6G-3 |
| — | — | 1938 | | GSG-2G-8 | GSG-3G-7 | GSG-4G-6 | GSG-5G-5 | GSG-6G-4 |
| — | — | 2300 | | | GSG-3G-8 | GSG-4G-7 | GSG-5G-6 | GSG-6G-5 |

Similarly, other glucose substitutes can be incorporated into the GSG, such as for example, rhamnose or deoxyhexose (see Table C) below. Table C depicts GSG groups corresponding to parental SGs with glucose (“G”; i.e., 2nd G after hyphen) and one moiety of rhamnose or deoxyhexose (“R”) added thereto.

TABLE C

| | | | GSG group based on parental SG-group given | | | | | |
|---|---|---|---|---|---|---|---|---|
| Parental SG | | | mw = 626 | mw = 788 | mw = 950 | mw = 1112 | mw = 1274 | mw = 1436 |
| Steviol-glycoside (GS) | SG-group | mw | SG-1G1R | SG-2G1R | SG-3G1R | SG-4G1R | SG-5G1R | SG-6G1R |
| Dulcoside A1 | SG-1G1R | 626 | | | | | | |
| Dulcoside A | SG-2G1R | 788 | GSG-1G1R-1 | | | | | |
| Dulcoside B (JECFA C) | | | | | | | | |
| SG-3 | | | | | | | | |
| Stevioside D | | | | | | | | |
| Reb C | SG-3G1R | 950 | GSG-1G1R-2 | GSG-2G1R-1 | | | | |
| Reb C2/Reb S | | | | | | | | |
| Stevioside E (SG-9) | | | | | | | | |
| Stevioside E2 | | | | | | | | |
| SG-10 | | | | | | | | |
| Reb L1 | | | | | | | | |
| SG-2 | | | | | | | | |
| SG-12 | SG-4G1R | 1112 | G5G-1G1R-3 | GSG-2G1R-2 | GSG-3G1R-1 | | | |
| Reb H | | | | | | | | |
| Reb J | | | | | | | | |
| Reb K | | | | | | | | |
| Reb K2 | | | | | | | | |
| SG-Unk4 | | | | | | | | |
| SG-Unk5 | | | | | | | | |
| Reb N | SG5-G1R | 1274 | GSG-1G1R-4 | GSG-2G1R-3 | GSG-3G1R-2 | GSG-4GLR-1 | | |
| Reb O | SG-6G1R | 1436 | GSG-1G1R-5 | GSG-2G1R-4 | GSG-3G1R-3 | GSG-4GLR-2 | GSG-5G1R-1 | |
| Reb O2 | | | | | | | | |
| — | — | 1598 | GSG-1G1R-6 | GSG-2G1R-5 | GSG-3G1R-4 | GSG-4G1R-3 | GSG-5G1R-2 | GSG-6G1R-1 |
| — | — | 1760 | GSG-1G1R-7 | GSG-2G1R-6 | GSG-3G1R-5 | GSG-4G1R-4 | GSG-5G1R-3 | GSG-6G1R-2 |
| — | — | 1922 | GSG-1G1R-8 | GSG-2G1R-7 | GSG-3G1R-6 | GSG-4G1R-5 | GSG-5G1R-4 | GSG-6G1R-3 |
| — | — | 2084 | | GSG-2G1R-8 | GSG-3G1R-7 | GSG-4G1R-6 | GSG-5G1R-5 | GSG-6G1R-4 |
| — | — | 2246 | | | GSG-3G1R-8 | GSG-4G1R-7 | GSG-5G1R-6 | GSG-6G1R-5 |

Different sugar donors such as glucose, xylose, rhamnose, etc. could be obtained during degradation of different compositions of stevia glycosides. These combinations of sugar donors could react with different amino acid donors, thus creating many unique and surprisingly pleasant flavors. The reaction removes the typical grassy, bitter, void, lingering and/or aftertaste of stevia glycosides.

In one embodiment, glycosylated steviol glycosides (GSGs) can be obtained for example, by synthetic manipulation or by enzymatic processes. The GSGs obtained by these methods are not naturally occurring steviol glycosides. The methods and GSGs found in KR10-2008-0085811 are herein incorporated by reference. Stevioside G1 (ST-G1), Stevioside G2 (ST-G2), Stevioside G3 (ST-G3), Stevioside G4 (ST-G4), Stevioside G5 (ST-G5), Stevioside G6 (ST-G6), Stevioside G7 (ST-G7), Stevioside G8 (ST-G8), Stevioside G9 (ST-G9), Rebaudioside A G1 (RA-G1), Rebaudioside A G2 (RA-G2), Rebaudioside A G3 (RA-G3), Rebaudioside A G4 (RA-G4), Rebaudioside A G5 (RA-G5), Rebaudioside A G6 (RA-G6), Rebaudioside A G7 (RA-G7), Rebaudioside A G8 (RA-G8), Rebaudioside A G9 (RA-G9), Rebaudioside B G1 (RB-G1), Rebaudioside B G2 (RB-G2), Rebaudioside B G3 (RB-G3), Rebaudioside B G4 (RB-G4), Rebaudioside B G5 (RB-G5), Rebaudioside B G6 (RB-G6), Rebaudioside B G7 (RB-G7), Rebaudioside B G8 (RB-G8), Rebaudioside B G9 (RB-G9), Rebaudioside C G1 (RC-G1), Rebaudioside C G2 (RC-G2), Rebaudioside C G3 (RC-G3), Rebaudioside C G4 (RC-G4), Rebaudioside C G5 (RC-G5), Rebaudioside C G6 (RC-G6), Rebaudioside C G7 (RC-G7), Rebaudioside C G8 (RC-G8), Rebaudioside C G9 (RC-G9), or any combination thereof can be incorporated into the sweetener compositions of the current invention. Alternatively in the current embodiments, the glycosylation process can be modified as to provide partially glycosylated steviol glycosides that can have further unique taste profiles.

A suitable method to prepare the glycosylated steviol glycosides (GSGs) can be found, for example, in KR10-2008-0085811 in Examples 1 and 2. It is also anticipated that other steviol glycosides, for example related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1 can be enzymatically modified to afford their corresponding multiple glycosylated glycosides

In a particular aspect, GSG-RA20, GSG-RA30, GSG-RA40, GSG-RA50, GSG-RA60, GSG-RA70, GSG-RA80, GSG-RA90, GSG-RA95, GSG-RA97, GSG-(RA50+RB8), GSG-(RA30+RC15), and GSG-(RA40+RB8) are GSGs which are used to be combined with steviol glycosides, such as RA, RB, RD, etc. GSG-RA20 is typically prepared from RA20 as a key starting material, GSG-RA30 is typically prepared from RA30 as a key starting material, GSG-RA40 is typically prepared from RA40 as a key starting material, GSG-RA50 is typically prepared from RA50 as a key starting material, GSG-RA60 is typically prepared from RA60 as a key starting material, GSG-RA70 is typically prepared from RA70 as a key starting material, GSG-RA80 is prepared from RA80 as the key starting material, GSG-RA90 is typically prepared from RA90 as a key starting material, GSG-RA95 is typically prepared from RA95 as a key starting material, and GSG-RA97 is prepared from RA97 as a key starting material. Since each composition contains varying concentrations of GSGs and steviol glycosides, then each composition may have different taste profiles. It is envisioned that specific ratios of GSGs and steviol glycosides may have unique and beneficial physical and chemical properties that are unknown and have not been previously disclosed.

All of the components of the compositions disclosed herein can be purchased or be made by processes known to those of ordinary skill in the art and combined (e.g., precipitation/co-precipitation, mixing, blending, grounding, mortar and pestle, microemulsion, solvothermal, sonochemical, etc.) or treated as defined by the current invention. Specifically, as examples, any one or more of GSG-RA20, GSG-RA30, GSG-RA40, GSG-RA50, GSG-RA60, GSG-RA70, GSG-RA80, GSG-RA90, GSG-RA95, GSG-RA97, GSG-(RA50+RB8), GSG-(RA30+RC15), and GSG-(RA40+RB8) can be combined with one or more of steviol, stevioside, steviolbioside, rebaudioside A, rebaudioside B, rebaudioside C, rebaudioside D, rebaudioside E, rebaudioside F, rebaudioside M, rebaudioside O, rebaudioside H, rebaudioside I, rebaudioside L, rebaudioside N, rebaudioside K, rebaudioside J, SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1 to provide suitable sweetening agent compositions. The content of GSG or GSGs from any one or more of GSG-RA20, GSG-RA30, GSG-RA40, GSG-RA50, GSG-RA60, GSG-RA70, GSG-RA80, GSG-RA90, GSG-RA95, GSG-RA97, GSG-(RA50+RB8), GSG-(RA30+RC15), and GSG-(RA40+RB8) mixed with the disclosed steviol glycosides such as the steviol glycosides found in the stevia plant or sweet tea extract can be from 1% wt/wt to 100% wt/wt. A GSG or GSGs, such as any one or more of GSG-RA20, GSG-RA30, GSG-RA40, GSG-RA50, GSG-RA60, GSG-RA70, GSG-RA80, GSG-RA90, GSG-RA95, GSG-RA97, GSG-(RA50+RB8), GSG-(RA30+RC15), and GSG-(RA40+RB8) can be included in the compositions described herein at 1% wt/wt, 2% wt/wt, 3% wt/wt, 4% wt/wt, 5% wt/wt, 6% wt/wt, 7% wt/wt, 8% wt/wt. 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example less than about 70 percentage by weight, less than about 50 percentage by weight, from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, from about 10% wt/wt to about 20% wt/wt, from about 20 to less than about 50 percentage by weight, from about 30 to less than about 50 percentage by weight, from about 40 to less than about 50 percentage by weight, and from about 20 to 45 percentage by weight of the sweetening agent composition.

In another aspect the SG's included in Table A having a molecular weight greater than 965, are contained in the sweetening agent composition. These steviol glycosides of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

In another aspect, the one or more mogrosides (MGs) are contained in the compositions described herein. The MGs of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

In another aspect, the one or more glycosylated steviol glycosides (GSGs) are contained in the composition described herein. The GSGs of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

In another aspect, the one or more glycosylated mogrosides (GMGs) are contained in the compositions described herein. The GMGs of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

In another aspect, the one or more low molecular weight SG(s) (“LMWSGs”), SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1, are included in the compositions described herein.

In particular, the LMWSGs having molecular weights less than or equal to 787 include related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3 and stevioside D.

The LMWSG(s), those having a molecular weight of less than or equal to 965, more particularly less than or equal to a molecular weight of 787, of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

In another aspect, the one or more glycosylated sweet tea glycosides are contained in the composition described herein. The glycosylated sweet tea glycosides of the compositions can make up 1% wt/wt, 2% wt/wt, 3% wt/wt, 4% wt/wt, 5% wt/wt, 6% wt/wt, 7% wt/wt, 8% wt/wt, 9% wt/wt, 10% wt/wt, 11% wt/wt, 12% wt/wt, 13% wt/wt, 14% wt/wt, 15% wt/wt, 16% wt/wt, 17% wt/wt, 18% wt/wt, 19% wt/wt, 20% wt/wt, 21% wt/wt, 22% wt/wt, 23% wt/wt, 24% wt/wt, 25% wt/wt, 26% wt/wt, 27% wt/wt, 28% wt/wt, 29% wt/wt, 30% wt/wt, 31% wt/wt, 32% wt/wt, 33% wt/wt, 34% wt/wt, 35% wt/wt, 36% wt/wt, 37% wt/wt, 38% wt/wt, 39% wt/wt, 40% wt/wt, 41% wt/wt, 42% wt/wt, 43% wt/wt, 44% wt/wt, 45% wt/wt, 46% wt/wt, 47% wt/wt, 48% wt/wt, 49% wt/wt, 50% wt/wt, 51% wt/wt, 52% wt/wt, 53% wt/wt, 54% wt/wt, 55% wt/wt, 56% wt/wt, 57% wt/wt, 58% wt/wt, 59% wt/wt, 60% wt/wt, 61% wt/wt, 62% wt/wt, 63% wt/wt, 64% wt/wt, 65% wt/wt, 66% wt/wt, 67% wt/wt, 68% wt/wt, 69% wt/wt, 70% wt/wt, 71% wt/wt, 72% wt/wt, 73% wt/wt, 74% wt/wt, 75% wt/wt, 76% wt/wt, 77% wt/wt, 78% wt/wt, 79% wt/wt, 80% wt/wt, 81% wt/wt, 82% wt/wt, 83% wt/wt, 84% wt/wt, 85% wt/wt, 86% wt/wt, 87% wt/wt, 88% wt/wt, 89% wt/wt, 90% wt/wt, 91% wt/wt, 92% wt/wt, 93% wt/wt, 94% wt/wt, 95% wt/wt, 96% wt/wt, 97% wt/wt, 98% wt/wt, 99% wt/wt, or 100% wt/wt and all ranges between 1 and 100% wt/wt, for example from about 1% wt/wt to about 99% wt/wt, from about 1% wt/wt to about 98% wt/wt, from about 1% wt/wt to about 97% wt/wt, from about 1% wt/wt to about 95% wt/wt, from about 1% wt/wt to about 90% wt/wt, from about 1% wt/wt to about 80% wt/wt, from about 1% wt/wt to about 70% wt/wt, from about 1% wt/wt to about 60% wt/wt, from about 1% wt/wt to about 50% wt/wt, from about 1% wt/wt to about 40% wt/wt, from about 1% wt/wt to about 30% wt/wt, from about 1% wt/wt to about 20% wt/wt, from about 1% wt/wt to about 10% wt/wt, from about 1% wt/wt to about 5% wt/wt, from about 2% wt/wt to about 99% wt/wt, from about 2% wt/wt to about 98% wt/wt, from about 2% wt/wt to about 97% wt/wt, from about 2% wt/wt to about 95% wt/wt, from about 2% wt/wt to about 90% wt/wt, from about 2% wt/wt to about 80% wt/wt, from about 2% wt/wt to about 70% wt/wt, from about 2% wt/wt to about 60% wt/wt, from about 2% wt/wt to about 50% wt/wt, from about 2% wt/wt to about 40% wt/wt, from about 2% wt/wt to about 30% wt/wt, from about 2% wt/wt to about 20% wt/wt, from about 2% wt/wt to about 10% wt/wt, from about 2% wt/wt to about 5% wt/wt, from about 3% wt/wt to about 99% wt/wt, from about 3% wt/wt to about 98% wt/wt, from about 3% wt/wt to about 97% wt/wt, from about 3% wt/wt to about 95% wt/wt, from about 3% wt/wt to about 90% wt/wt, from about 3% wt/wt to about 80% wt/wt, from about 3% wt/wt to about 70% wt/wt, from about 3% wt/wt to about 60% wt/wt, from about 3% wt/wt to about 50% wt/wt, from about 3% wt/wt to about 40% wt/wt, from about 3% wt/wt to about 30% wt/wt, from about 3% wt/wt to about 20% wt/wt, from about 3% wt/wt to about 10% wt/wt, from about 3% wt/wt to about 5% wt/wt, from about 5% wt/wt to about 99% wt/wt, from about 5% wt/wt to about 98% wt/wt, from about 5% wt/wt to about 97% wt/wt, from about 5% wt/wt to about 95% wt/wt, from about 5% wt/wt to about 90% wt/wt, from about 5% wt/wt to about 80% wt/wt, from about 5% wt/wt to about 70% wt/wt, from about 5% wt/wt to about 60% wt/wt, from about 5% wt/wt to about 50% wt/wt, from about 5% wt/wt to about 40% wt/wt, from about 5% wt/wt to about 30% wt/wt, from about 5% wt/wt to about 20% wt/wt, from about 5% wt/wt to about 10% wt/wt, from about 10% wt/wt to about 99% wt/wt, from about 10% wt/wt to about 98% wt/wt, from about 10% wt/wt to about 97% wt/wt, from about 10% wt/wt to about 95% wt/wt, from about 10% wt/wt to about 90% wt/wt, from about 10% wt/wt to about 80% wt/wt, from about 10% wt/wt to about 70% wt/wt, from about 10% wt/wt to about 60% wt/wt, from about 10% wt/wt to about 50% wt/wt, from about 10% wt/wt to about 40% wt/wt, from about 10% wt/wt to about 30% wt/wt, and from about 10% wt/wt to about 20% wt/wt, of the sweetening composition.

It should be understood that ranges for ratios include the numbers noted as well as all values there between. For example, the ratio of 1:99 to 99:1 includes 1 and 99 at the endpoints and all values there between such as 1:99, 2:98, 3:97, 4:96, 5:95, 6:94, 7:93, 8:92, 9:91, 10:90, 11:89, 12:88, 13:87, 14:86, 15:85, 16:84:17:83, 18:82, 19:81, 20:80, 21:79, 22:78, 23:77, 24:76, 25:75; 26:74, 27:73, 28:72, 29:71, 30:70, 31:69, 32:68, 33:67, 34:66, 35:65, 36:64, 37:63, 38:62, 39:61, 40:60, 41:59, 42:58, 43:57, 44:56, 45:55, 46:54, 47:53, 48:52, 49:51, 50:50, 51:49, 52:48, 53:47, 54:46, 55:45, 56:44, 57:43, 58:42, 59:41, 60:40, 61:39, 62:38, 63:37, 64:36, 65:35, 66:34, 67:33, 68:32, 69:31, 70:30, 71:29, 72:28, 73:27, 74:26, 75:25, 76:24, 77:23, 78:22, 79:21, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2 and 99:1.

Similarly, the ratio of 20:1 to 5:1 includes ratios of 20:1, 19:1, 18:1, 17:1, 16:1, 15:1, 14:1, 13:1, 12:1, 11:1, 10:1, 9:1, 8:1, 7:1, 6:1 and 5:1. Ratios of 1:10 to about 1:1 include 1:10, 2:8, 3:7, 4:6, 5:5 (which is 1:1). Ratios of 10:1 to 1:1 include 10:1, 9:1, 8:1, 7:1, 6:1, 5:1, 4:1, 3:1, 2:1 and 1:1.

It should be noted that the percentages provided above include compositions of combinations of sweetening agents disclosed herein, including low molecular weight SG having a molecular weight of less than or equal to 965 daltons, more particularly a molecular weight less than or equal to 787, sweet tea extracts, sweet tea components, such as rubusoside and suaviosides, glycosylated sweet tea extracts, SG's, GSG's, MG's, GMG's, and mixtures thereof are part of the composition. The weight ratio of low molecular weight SGs, having a molecular weight of less than or equal to 965, more particularly a molecular weight of less than or equal to 787, to other components can range from 100:0.1 to 0.1:100 and all values there between. That is, for example, where a non-low molecular weight SG comprises 90% by weight of the composition, up to 10% by weight of the composition can be a low molecular weight SG, e.g., 90:10 or 9:1. Another example would be where 99% of the composition is a non-low molecular weight SG and 1% by weight would be a low molecular weight SG, having a molecular weight less than or equal to 965, 787, etc., e.g., 99:1, for use in producing sweetening agent compositions.

In another aspect, a sweetener composition of the present application includes one or more low molecular weight SG(s) having a molecular weight equal to or less than 965, more particularly a molecular weight less than or equal to 787, with one or more of sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products, and mixtures thereof and optionally, containing other non-SG or non-MG sweeteners and/or additional additives as further described below.

The terms “non-SG sweetener” and “non-MG sweetener” include, but are not limited to, natural sweeteners, natural high potency sweeteners, synthetic sweeteners, or a combination thereof that are not derived from sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products and does not include low molecular weight SG(s) having a molecular weight equal to or less than 965, more particularly a molecular weight less than or equal to 787.

As used herein, a “natural sweetener” refers to any sweetener found naturally in nature, excluding sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products and does not include low molecular weight SG(s) having a molecular weight equal to or less than 965, more particularly a molecular weight less than or equal to 787. The phrase “natural high potency sweetener” refers to any sweetener found naturally in nature that has a sweetness potency greater than sucrose, fructose, or glucose, yet has less calories. The phrase “synthetic sweetener” refers to any composition which is not found naturally in nature that has a sweetness potency greater than sucrose, fructose, or glucose, yet has less calories. As used herein, the terms “natural sweeteners,” “natural high potency sweeteners” and “synthetic sweeteners” do not include sweet tea extracts, stevia extracts, swingle extracts, glycosylated sweet tea extracts, glycosylated stevia extracts, glycosylated swingle extracts, glycosylated sweet tea glycosides, glycosylated steviol glycosides, glycosylated mogrosides, as well as the individual components of the extracts or glycosylated products and does not include low molecular weight SG(s) having a molecular weight equal to or less than 965, more particularly a molecular weight less than or equal to 787.

In certain embodiments, the non-SG and non-MG sweetener includes at least one carbohydrate sweetener. Exemplary carbohydrate sweeteners are selected from, but not limited to, the group consisting of sucrose, glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, fucose, rhamnose, arabinose, turanose, sialose and combinations thereof.

Other suitable non-SG/non-MG sweeteners include monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, DOLCIA PRIMA™, allulose, inulin, neohesperidine dihydrochalcone (NHDC) and combinations thereof.

The non-SG/non-MG sweetener may be a caloric sweetener or mixture of caloric sweeteners. Caloric sweeteners include sucrose, fructose, glucose, high fructose corn/starch syrup, a beet sugar, a cane sugar, and combinations thereof.

In certain embodiments, the non-SG/non-MG sweetener is a rare sugar selected from sorbose, lyxose, ribulose, xylose, xylulose, D-allose, L-ribose, D-tagatose, L-glucose, L-fucose, L-arabinose, turanose and combinations thereof.

The one or more non-SG/non-MG sweetener of the sweetener compositions of the present application can make up anywhere from about 0.1 wt. % of the sweetener composition to about 80 wt. % of the sweetener composition, specifically about 0.01 wt. %, about 0.02 wt %, about 0.05 wt %, about 0.07 wt %, about 0.1 wt %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, about 55 wt. %, about 56 wt. %, about 57 wt. %, about 58 wt. %, about 59 wt. %, about 60 wt. %, about 61 wt. %, about 62 wt. %, about 63 wt. %, about 64 wt. %, about 65 wt. %, about 66 wt. %, about 67 wt. %, about 68 wt. %, about 69 wt. %, about 70 wt. %, about 71 wt. %, about 72 wt. %, about 73 wt. %, about 74 wt. %, about 75 wt. %, about 76 wt. %, about 77 wt. %, about 78 wt. %, about 79 wt. %, about 80 wt. %, and all ranges there between, including for example from about 0.01 wt % to about 20 wt %, about 0.03 wt % to about 20 wt %, about 0.05 wt % to about 20 wt %, about 0.07 wt % to about 20 wt %, about 0.1 wt % to about 20 wt %, about 0.3 wt % to about 20 wt %, about 0.5 wt % to about 20 wt %, about 0.7 wt % to about 20 wt %, about 1 wt % to about 20 wt %, about 3 wt % to about 20 wt %, about 5 wt % to about 20 wt %, about 7 wt % to about 20 wt %, about 10 wt % to about 20 wt %, about 15 wt % to about 20 wt %, about 0.01 wt % to about 10 wt %, about 0.03 wt % to about 10 wt %, about 0.05 wt % to about 10 wt %, about 0.07 wt % to about 10 wt %, about 0.1 wt % to about 10 wt %, about 0.3 wt % to about 10 wt %, about 0.5 wt % to about 10 wt %, about 0.7 wt % to about 10 wt %, about 1 wt % to about 10 wt %, about 3 wt % to about 10 wt %, about 5 wt % to about 10 wt %, about 7 wt % to about 10 wt %, about 0.01 wt % to about 5 wt %, about 0.03 wt % to about 5 wt %, about 0.05 wt % to about 5 wt %, about 0.07 wt % to about 5 wt %, about 0.1 wt % to about 5 wt %, about 0.3 wt % to about 5 wt %, about 0.5 wt % to about 5 wt %, about 0.7 wt % to about 5 wt %, about 1 wt % to about 5 wt %, about 3 wt % to about 5 wt %, about 0.01 wt % to about 2.5 wt %, about 0.03 wt % to about 2.5 wt %, about 0.05 wt % to about 2.5 wt %, about 0.07 wt % to about 2.5 wt %, about 0.1 wt % to about 2.5 wt %, about 0.3 wt % to about 2.5 wt %, about 0.5 wt % to about 2.5 wt %, about 0.7 wt % to about 2.5 wt %, about 1 wt % to about 2.5 wt %, about 5 wt. % to about 30 wt. %, from about 10 wt. % to about 30 wt. %, from about 20 wt. % to about 40 wt. %, or from about 30 wt. % to about 50 wt. %.

In other embodiments, the sweetener compositions of the present application further includes one or more additional additives selected from the group consisting of flavoring agents, salts, minerals, organic acids and inorganic acids, polyols, nucleotides, bitter compounds, astringent compounds, proteins or protein hydrolysates, surfactants, gums and waxes, antioxidants, polymers, fatty acids, vitamins, preservatives, hydration agents, probiotics/prebiotics, weight management agent and combinations thereof, as further described below.

As used herein, a “flavoring agent” or “flavorant” herein refers to a compound or an ingestibly acceptable salt or solvate thereof that induces a flavor or taste in an animal or a human. The flavoring agent can be natural, semi-synthetic, or synthetic. Suitable flavorants and flavoring ingredient additives for use in the SG compositions of the present application include, but are not limited to, vanillin, vanilla extract, mango extract, cinnamon, citrus, coconut, ginger, viridiflorol, almond, bay, thyme, cedar leaf, nutmeg, allspice, sage, mace, menthol (including menthol without mint), an essential oil, such as an oil derived from a plant or a fruit, such as peppermint oil, spearmint oil, other mint oils, clove oil, cinnamon oil, oil of wintergreen, or an oil of almonds; a plant extract, fruit extract or fruit essence from grape skin extract, grape seed extract, apple, banana, watermelon, pear, peach, grape, strawberry, raspberry, cherry, plum, pineapple, apricot, a flavoring agent comprising a citrus flavor, such as an extract, essence, or oil of lemon, lime, orange, tangerine, grapefruit, citron, kumquat, and combinations thereof.

Non-limiting examples of proprietary flavorants include Dohler™ Natural Flavoring Sweetness Enhancer K14323 (Dohler™, Darmstadt, Germany), Symrise™ Natural Flavor Mask for Sweeteners 161453 and 164126 (Symrise™, Holzminden, Germany), Natural Advantage™ Bitterness Blockers 1, 2, 9 and 10 (Natural Advantage™, Freehold, N.J., U.S.A.), and Sucramask™ (Creative Research Management, Stockton, Calif., U.S.A.).

In some embodiments, the flavoring agent is present in the sweetener composition of the present application in a concentration from about 0.1 ppm to about 4,000 ppm.

One or more salts may be included in the sweetener composition of the present application. The salts may be organic salts or inorganic salts. As used herein, the term “salt” refers to salts that retain the desired chemical activity of the sweetener compositions of the present application and are safe for human or animal consumption in a generally acceptable range.

In some embodiments, the one or more salts are salts formed with metal cations such as calcium, bismuth, barium, magnesium, aluminum, copper, cobalt, nickel, cadmium, sodium, potassium, and the like, or with a cation formed from ammonia, N,N-dibenzylethylenediamine, D-glucosamine, ethanolamine, diethanolamine, triethanolamine, N-methylglucamine tetraethylammonium, or ethylenediamine.

In some embodiments, the one or more salts are formed with inorganic acids, such as hydrochloric acid, hydrobromic acid, sulfuric acid, nitric acid, phosphoric acid, and the like; or formed with organic acids, such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid.

In some embodiments, inorganic salts include, but are not limited to, sodium chloride, sodium carbonate, sodium bicarbonate, sodium acetate, sodium sulfide, sodium sulfate, sodium phosphate, potassium chloride, potassium citrate, potassium carbonate, potassium bicarbonate, potassium acetate, europium chloride (EuCl₃), gadolinium chloride (GdCl₃), terbium chloride (TbCl₃), magnesium sulfate, alum, magnesium chloride, mono-, di-, tri-basic sodium or potassium salts of phosphoric acid (e.g., inorganic phosphates), salts of hydrochloric acid (e.g., inorganic chlorides), sodium carbonate, sodium bisulfate, and sodium bicarbonate. Suitable organic salts include, but are not limited to, choline chloride, alginic acid sodium salt (sodium alginate), glucoheptonic acid sodium salt, gluconic acid sodium salt (sodium gluconate), gluconic acid potassium salt (potassium gluconate), guanidine HCl, glucosamine HCl, amiloride HCl, monosodium glutamate (MSG), adenosine monophosphate salt, magnesium gluconate, potassium tartrate (monohydrate), and sodium tartrate (dihydrate).

In certain embodiments, the salt is a metal or metal alkali halide, a metal or metal alkali carbonate or bicarbonate, or a metal or metal alkali phosphate, biphosphate, pyrophosphate, triphosphate, metaphosphate, or metabisulfate thereof. In certain particular embodiments, the salt is an inorganic salt that comprises sodium, potassium, calcium, or magnesium. In some embodiments, the salt is a sodium salt or a potassium salt.

Alternative salts include various chloride or sulfate salts, such as sodium chloride, potassium chloride, magnesium chloride, sodium sulfate, magnesium sulfate, and potassium sulfate, or any edible salt. In some embodiments, the one or more salts comprise one or more SG, MG, GSG, or GMG salts. Additionally, the low molecular weight SG having a molecular weight of less than or equal 965, more particularly a molecular weight less than or equal to 787, can also be in salt form.

Suitable LMWSG acids (having a carboxyl, COOH group) for the preparation of corresponding LMWSG salts include steviol-monoside, SG-4, dulcoside A1, iso-steviolbioside, reb-G1, steviolbioside, reb-F1, reb-R1, dulcoside, SG-3, iso-reb B, reb B and reb L1. The one or more salts can make up anywhere from about 0.01 wt. % of the sweetener composition to about 50 wt. % of the sweetener composition, specifically about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, and all ranges there between, including for example from about 0.01 wt % to about 10 wt %, about 0.03 wt % to about 10 wt %, about 0.05 wt % to about 10 wt %, about 0.07 wt % to about 10 wt %, about 0.1 wt % to about 10 wt %, about 0.3 wt % to about 10 wt %, about 0.5 wt % to about 10 wt %, about 0.7 wt % to about 10 wt %, about 1 wt % to about 10 wt %, about 3 wt % to about 10 wt %, about 5 wt % to about 10 wt %, about 7 wt % to about 10 wt %, about 0.01 wt % to about 3 wt %, about 0.03 wt % to about 3 wt %, about 0.05 wt % to about 3 wt %, about 0.07 wt % to about 3 wt %, about 0.1 wt % to about 3 wt %, about 0.3 wt % to about 3 wt %, about 0.5 wt % to about 3 wt %, about 0.7 wt % to about 3 wt %, about 1 wt % to about 3 wt %, about 0.01 wt % to about 1 wt %, about 0.03 wt % to about 1 wt %, about 0.05 wt % to about 1 wt %, about 0.07 wt % to about 1 wt %, about 0.1 wt % to about 1 wt %, about 0.3 wt % to about 1 wt %, about 0.5 wt % to about 1 wt %, about 0.7 wt % to about 1 wt %, about 0.01 wt % to about 0.3 wt %, about 0.03 wt % to about 0.3 wt %, about 0.05 wt % to about 0.3 wt %, about 0.07 wt % to about 0.3 wt %, about 0.1 wt % to about 0.3 wt %, about 0.01 wt % to about 0.1 wt %, about 0.03 wt % to about 0.1 wt %, about 0.05 wt % to about 0.1 wt %, about 0.07 wt % to about 0.1 wt %, about 0.01 wt % to about 0.03 wt %, about 0.01 wt % to about 0.05 wt %, about 0.01 wt % to about 0.07 wt %, about 5 wt. % to about 30 wt. %, from about 10 wt. % to about 30 wt. %, or from about 20 wt. % to about 30 wt. %.

Alternatively, the LMWSG carboxylic acids including steviol-monoside, SG-4, dulcoside A1, iso-steviolbioside, reb-G1, steviolbioside, reb-F1, reb-R1, dulcoside, SG-3, iso-reb B, reb B and reb L1 can make up anywhere from about 0.01 wt. % of the sweetener composition to about 50 wt. % of the sweetener composition, specifically about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, about 31 wt. %, about 32 wt. %, about 33 wt. %, about 34 wt. %, about 35 wt. %, about 36 wt. %, about 37 wt. %, about 38 wt. %, about 39 wt. %, about 40 wt. %, about 41 wt. %, about 42 wt. %, about 43 wt. %, about 44 wt. %, about 45 wt. %, about 46 wt. %, about 47 wt. %, about 48 wt. %, about 49 wt. %, about 50 wt. %, and all ranges there between, including for example from about 0.01 wt % to about 10 wt %, about 0.03 wt % to about 10 wt %, about 0.05 wt % to about 10 wt %, about 0.07 wt % to about 10 wt %, about 0.1 wt % to about 10 wt %, about 0.3 wt % to about 10 wt %, about 0.5 wt % to about 10 wt %, about 0.7 wt % to about 10 wt %, about 1 wt % to about 10 wt %, about 3 wt % to about 10 wt %, about 5 wt % to about 10 wt %, about 7 wt % to about 10 wt %, about 0.01 wt % to about 3 wt %, about 0.03 wt % to about 3 wt %, about 0.05 wt % to about 3 wt %, about 0.07 wt % to about 3 wt %, about 0.1 wt % to about 3 wt %, about 0.3 wt % to about 3 wt %, about 0.5 wt % to about 3 wt %, about 0.7 wt % to about 3 wt %, about 1 wt % to about 3 wt %, about 0.01 wt % to about 1 wt %, about 0.03 wt % to about 1 wt %, about 0.05 wt % to about 1 wt %, about 0.07 wt % to about 1 wt %, about 0.1 wt % to about 1 wt %, about 0.3 wt % to about 1 wt %, about 0.5 wt % to about 1 wt %, about 0.7 wt % to about 1 wt %, about 0.01 wt % to about 0.3 wt %, about 0.03 wt % to about 0.3 wt %, about 0.05 wt % to about 0.3 wt %, about 0.07 wt % to about 0.3 wt %, about 0.1 wt % to about 0.3 wt %, about 0.01 wt % to about 0.1 wt %, about 0.03 wt % to about 0.1 wt %, about 0.05 wt % to about 0.1 wt %, about 0.07 wt % to about 0.1 wt %, about 0.01 wt % to about 0.03 wt %, about 0.01 wt % to about 0.05 wt %, about 0.01 wt % to about 0.07 wt %, about 5 wt. % to about 30 wt. %, from about 10 wt. % to about 30 wt. %, or from about 20 wt. % to about 30 wt. %.

Minerals, in accordance with the teachings of this application, comprise inorganic chemical elements required by living organisms. Minerals are comprised of a broad range of compositions (e.g., elements, simple salts, and complex silicates) and also vary broadly in crystalline structure. They may naturally occur in foods and beverages, may be added as a supplement, or may be consumed or administered separately from foods or beverages.

Minerals may be categorized as either bulk minerals, which are required in relatively large amounts, or trace minerals, which are required in relatively small amounts. Bulk minerals generally are required in amounts greater than or equal to about 100 mg per day and trace minerals are those that are required in amounts less than about 100 mg per day.

In particular embodiments of the present application, the mineral is chosen from bulk minerals, trace minerals or combinations thereof. Non-limiting examples of bulk minerals include calcium, chlorine, magnesium, phosphorous, potassium, sodium, and sulfur. Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In certain particular embodiments, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

The minerals embodied herein may be in any form known to those of ordinary skill in the art. For example, in a particular embodiment the minerals may be in their ionic form, having either a positive or negative charge. In another particular embodiment the minerals may be in their molecular form. For example, sulfur and phosphorous often are found naturally as sulfates, sulfides, and phosphates.

Suitable organic acid additives include any compound which comprises a —COOH moiety, such as, for example, C2-C30 carboxylic acids, substituted hydroxyl C2-C30 carboxylic acids, butyric acid (ethyl esters), substituted butyric acid (ethyl esters), benzoic acid, substituted benzoic acids (e.g., 2,4-dihydroxybenzoic acid), substituted cinnamic acids, hydroxyacids, substituted hydroxybenzoic acids, anisic acid substituted cyclohexyl carboxylic acids, tannic acid, aconitic acid, lactic acid, tartaric acid, citric acid, isocitric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, creatine, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration.

The examples of the organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfinyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount effective to provide a concentration from about 10 ppm to about 5,000 ppm when present in an orally consumable composition, such as, for example, a beverage.

Organic acids also include amino acids such as, aspartic acid, arginine, glycine, glutamic acid, proline, threonine, threonine, cysteine, cystine, alanine, valine, tyrosine, leucine, arabinose, trans-4-hydroxyproline, isoleucine, asparagine, serine, lysine, histidine, ornithine, methionine, carnitine, aminobutyric acid (α-, β-, and/or δ-isomers), glutamine, hydroxyproline, taurine, norvaline and sarcosine. The amino acid may be in the D- or L-configuration and in the mono-, di-, or tri-form of the same or different amino acids. Additionally, the amino acids may be α-, β-, γ- and/or δ-isomers if appropriate. Combinations of the foregoing amino acids and their corresponding salts (e.g., sodium, potassium, calcium, magnesium salts or other alkali or alkaline earth metal salts thereof, or acid salts) also are suitable additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids.

As used herein, amino acids also encompass both peptides and polypeptides (e.g., dipeptides, tripeptides, tetrapeptides, and pentapeptides) such as glutathione and L-alanyl-L-glutamine. Suitable polyamino acid additives include poly-L-aspartic acid, poly-L-lysine (e.g., poly-L-α-lysine or poly-L-γ-lysine), poly-L-ornithine (e.g., poly-L-α-ornithine or poly-L-γ-ornithine), poly-L-arginine, other polymeric forms of amino acids, and salt forms thereof (e.g., calcium, potassium, sodium, or magnesium salts such as L-glutamic acid mono sodium salt). The poly-amino acid additives also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable additives in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-lysine with a MW of 1,500, MW of 6,000, MW of 25,200, MW of 63,000, MW of 83,000, or MW of 300,000.

In particular embodiments, the amino acid is present in the sweetener composition in an amount effective to provide a concentration from about 10 ppm to about 50,000 ppm when present in an orally consumable composition, such as, for example, a beverage. In another embodiment, the amino acid is present in the sweetener composition in an amount effective to provide a concentration from about 1,000 ppm to about 10,000 ppm when present in an orally consumable composition, such as, for example, from about 2,500 ppm to about 5,000 ppm or from about 250 ppm to about 7,500 ppm.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g., inositol hexaphosphate Mg/Ca).

The inorganic acid additive is present in the sweetener composition in an amount effective to provide a concentration from about 25 ppm to about 25,000 ppm when present in an orally consumable composition, such as, for example, a beverage.

The term “polyol,” as used herein, refers to a molecule that contains more than one hydroxyl group. A polyol may be a diol, triol, or a tetraol which contains 2, 3, and 4 hydroxyl groups respectively. A polyol also may contain more than 4 hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group.

Non-limiting examples of polyols in some embodiments include maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligo saccharides, reduced maltose syrup, reduced glucose syrup, and sugar alcohols or any other carbohydrates capable of being reduced which do not adversely affect taste.

In certain embodiments, the polyol is present in the sweetener compositions in an amount effective to provide a concentration from about 100 ppm to about 250,000 ppm when present in an orally consumable composition. In other embodiments, the polyol is present in the sweetener compositions in an amount effective to provide a concentration from about 400 ppm to about 80,000 ppm when present in an orally consumable composition, such as, for example, from about 5,000 ppm to about 40,000 ppm.

Suitable nucleotide additives include, but are not limited to, inosine monophosphate (“IMP”), guanosine monophosphate (“GMP”), adenosine monophosphate (“AMP”), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil).

The nucleotide is present in the sweetener composition in an amount effective to provide a concentration from about 5 ppm to about 1,000 ppm when present in an orally consumable composition, such as, for example, a beverage.

Suitable bitter compound additives include, but are not limited to, caffeine, quinine, urea, bitter orange oil, naringin, quassia, and salts thereof.

When present in a consumable, such as, for example, a beverage, the bitter compound is present in the sweetener composition in an amount effective to provide a concentration from about 25 ppm to about 25,000 ppm.

Suitable astringent compound additives include, but are not limited to, tannic acid, europium chloride (EuCl₃), gadolinium chloride (GdCl₃), terbium chloride (TbCl₃), alum, tannic acid, and polyphenols (e.g., tea polyphenols). The astringent additive is present in the sweetener composition in an amount effective to provide a concentration from about 10 ppm to about 5,000 ppm when present in a consumable, such as, for example, a beverage.

Suitable protein or protein hydrolysate additives include, but are not limited to, bovine serum albumin (BSA), whey protein (including fractions or concentrates thereof such as 90% instant whey protein isolate, 34% whey protein, 50%>hydrolyzed whey protein, and 80%>whey protein concentrate), soluble rice protein, soy protein, protein isolates, protein hydrolysates, reaction products of protein hydrolysates, glycoproteins, and/or proteoglycans containing amino acids (e.g., glycine, alanine, serine, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolysates (e.g., porcine collagen hydrolysate).

The protein hydrolysate is present in the sweetener composition in an amount effective to provide a concentration from about 200 ppm to about 50,000 ppm when present in a consumable, such as, for example, a beverage.

Suitable surfactant additives include, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltnmethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

The surfactant additive is present in the sweetener composition in an amount effective to provide a concentration from about 30 ppm to about 2,000 ppm when present in an orally consumable composition, such as, for example, a beverage.

Gums and mucilages represent a broad array of different branched structures. Guar gum, derived from the ground endosperm of the guar seed, is a galactomannan. Guar gum is commercially available (e.g., Benefiber by Novartis AG). Other gums, such as gum arabic and pectins, have still different structures. Still other gums include xanthan gum, gellan gum, tara gum, psyllium seed husk gum, and locust bean gum.

Waxes are esters of ethylene glycol and two fatty acids, generally occurring as a hydrophobic liquid that is insoluble in water.

As used herein “antioxidant” refers to any substance which inhibits, suppresses, or reduces oxidative damage to cells and biomolecules. Without being bound by theory, it is believed that antioxidants inhibit, suppress, or reduce oxidative damage to cells or biomolecules by stabilizing free radicals before they can cause harmful reactions. As such, antioxidants may prevent or postpone the onset of some degenerative diseases.

Examples of suitable antioxidants for embodiments of this application include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, and combinations thereof. In some embodiments, the antioxidant is vitamin A, vitamin C, vitamin E, ubiquinone, mineral selenium, manganese, melatonin, a-carotene, β-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone, phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-a-lipoic acid, N-acetylcysteine, emblicanin, apple extract, apple skin extract (applephenon), rooibos extract red, rooibos extract, green, hawthorn berry extract, red raspberry extract, green coffee antioxidant (GCA), aronia extract 20%, grape seed extract (VinOseed), cocoa extract, hops extract, mangosteen extract, mangosteen hull extract, cranberry extract, pomegranate extract, pomegranate hull extract, pomegranate seed extract, hawthorn berry extract, pomella pomegranate extract, cinnamon bark extract, grape skin extract, bilberry extract, pine bark extract, pycnogenol, elderberry extract, mulberry root extract, wolfberry (gogi) extract, blackberry extract, blueberry extract, blueberry leaf extract, raspberry extract, turmeric extract, citrus bioflavonoids, black currant, ginger, acai powder, green coffee bean extract, green tea extract, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this application include, but are not limited to, fruits, vegetables, tea, cocoa, chocolate, spices, herbs, rice, organ meats from livestock, yeast, whole grains, or cereal grains.

Particular antioxidants belong to the class of phytonutrients called polyphenols (also known as “polyphenolics”), which are a group of chemical substances found in plants, characterized by the presence of more than one phenol group per molecule. A variety of health benefits may be derived from polyphenols, including prevention of cancer, heart disease, and chronic inflammatory disease and improved mental strength and physical strength, for example. Suitable polyphenols for embodiments of this application include catechins, proanthocyanidins, procyanidins, anthocyanins, quercerin, rutin, reservatrol, isoflavones, curcumin, punicalagin, ellagitannin, hesperidin, naringin, citrus flavonoids, chlorogenic acid, other similar materials, and combinations thereof.

In particular embodiments, the antioxidant is a catechin such as, for example, epigallocatechin gallate (EGCG). Suitable sources of catechins for embodiments of this application include, but are not limited to, green tea, white tea, black tea, oolong tea, chocolate, cocoa, red wine, grape seed, red grape skin, purple grape skin, red grape juice, purple grape juice, berries, pycnogenol, and red apple peel.

In some embodiments, the antioxidant is chosen from proanthocyanidins, procyanidins or combinations thereof. Suitable sources of proanthocyanidins and procyanidins for embodiments of this application include, but are not limited to, red grapes, purple grapes, cocoa, chocolate, grape seeds, red wine, cacao beans, cranberry, apple peel, plum, blueberry, black currants, choke berry, green tea, sorghum, cinnamon, barley, red kidney bean, pinto bean, hops, almonds, hazelnuts, pecans, pistachio, pycnogenol, and colorful berries.

In particular embodiments, the antioxidant is an anthocyanin. Suitable sources of anthocyanins for embodiments of this application include, but are not limited to, red berries, blueberries, bilberry, cranberry, raspberry, cherry, pomegranate, strawberry, elderberry, choke berry, red grape skin, purple grape skin, grape seed, red wine, black currant, red currant, cocoa, plum, apple peel, peach, red pear, red cabbage, red onion, red orange, and blackberries.

In some embodiments, the antioxidant is chosen from quercetin, rutin or combinations thereof. Suitable sources of quercetin and rutin for embodiments of this application include, but are not limited to, red apples, onions, kale, bog whortleberry, lingonberrys, chokeberry, cranberry, blackberry, blueberry, strawberry, raspberry, black currant, green tea, black tea, plum, apricot, parsley, leek, broccoli, chili pepper, berry wine, and ginkgo.

In some embodiments, the antioxidant is reservatrol. Suitable sources of reservatrol for embodiments of this application include, but are not limited to, red grapes, peanuts, cranberry, blueberry, bilberry, mulberry, Japanese Itadori tea, and red wine.

In particular embodiments, the antioxidant is an isoflavone. Suitable sources of isoflavones for embodiments of this application include, but are not limited to, soy beans, soy products, legumes, alfalfa sprouts, chickpeas, peanuts, and red clover.

In some embodiments, the antioxidant is curcumin. Suitable sources of curcumin for embodiments of this application include, but are not limited to, turmeric and mustard.

In particular embodiments, the antioxidant is chosen from punicalagin, ellagitannin or combinations thereof. Suitable sources of punicalagin and ellagitannin for embodiments of this application include, but are not limited to, pomegranate, raspberry, strawberry, walnut, and oak-aged red wine.

In some embodiments, the antioxidant is a citrus flavonoid, such as hesperidin or naringin. Suitable sources of citrus flavonoids, such as hesperidin or naringin, for embodiments of this application include, but are not limited to, oranges, grapefruits, and citrus juices.

In particular embodiments, the antioxidant is chlorogenic acid. Suitable sources of chlorogenic acid for embodiments of this application include, but are not limited to, green coffee, yerba mate, red wine, grape seed, red grape skin, purple grape skin, red grape juice, purple grape juice, apple juice, cranberry, pomegranate, blueberry, strawberry, sunflower, Echinacea, pycnogenol, and apple peel.

Suitable polymer additives include, but are not limited to, chitosan, pectin, pectic, pectinic, polyuronic, polygalacturonic acid, starch, food hydrocolloid or crude extracts thereof (e.g., gum acacia Senegal (Fibergum™) gum acacia seyal, carageenan), poly-L-lysine (e.g., poly-L-α-lysine or poly-L-ε-lysine), poly-L-ornithine (e.g., poly-L-α-ornithine or poly-L-ε-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers.

The polymer is present in the sweetener composition in an amount effective to provide a concentration from about 30 ppm to about 2,000 ppm when present in an orally consumable composition, such as, for example, a beverage.

As used herein, “fatty acid” refers to any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. As used herein, “omega-3 fatty acid” refers to any polyunsaturated fatty acid having a first double bond as the third carbon-carbon bond from the terminal methyl end of its carbon chain. In particular embodiments, the omega-3 fatty acid may comprise a long chain omega-3 fatty acid. As used herein, “omega-6 fatty acid” refers to any polyunsaturated fatty acid having a first double bond as the sixth carbon-carbon bond from the terminal methyl end of its carbon chain.

Suitable omega-3 fatty acids for use in embodiments of the present application can be derived from algae, fish, animals, plants, or combinations thereof, for example. Examples of suitable omega-3 fatty acids include, but are not limited to, linolenic acid, alpha-linolenic acid, eicosapentaenoic acid, docosahexaenoic acid, stearidonic acid, eicosatetraenoic acid and combinations thereof. In some embodiments, suitable omega-3 fatty acids can be provided in fish oils, (e.g., menhaden oil, tuna oil, salmon oil, bonito oil, and cod oil), microalgae omega-3 oils or combinations thereof. In particular embodiments, suitable omega-3 fatty acids may be derived from commercially available omega-3 fatty acid oils, such as Microalgae DHA oil (from Martek, Columbia, Md.), OmegaPure (from Omega Protein, Houston, Tex.), Marinol C-38 (from Lipid Nutrition, Channahon, Ill.), Bonito oil and MEG-3 (from Ocean Nutrition, Dartmouth, NS), Evogel (from Symrise, Holzminden, Germany), Marine Oil, from tuna or salmon (from Arista Wilton, Conn.), OmegaSource 2000, Marine Oil, from menhaden and Marine Oil, from cod (from OmegaSource, RTP, NC).

Suitable omega-6 fatty acids include, but are not limited to, linoleic acid, gamma-linolenic acid, dihommo-gamma-linolenic acid, arachidonic acid, eicosadienoic acid, docosadienoic acid, adrenic acid, docosapentaenoic acid and combinations thereof.

Suitable esterified fatty acids for embodiments of the present application may include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

Vitamins are organic compounds that the human body needs in small quantities for normal functioning. The body uses vitamins without breaking them down, unlike other nutrients such as carbohydrates and proteins. To date, thirteen vitamins have been recognized, and one or more can be used in the compositions herein. Suitable vitamins and their alternative chemical names are provided in the accompanying parentheses which follow include, vitamin A (retinol, retinaldehyde), vitamin D (calciferol, cholecalciferol, lumisterol, ergocalciferol, dihydrotachysterol, 7-dehydrocholesterol), vitamin E (tocopherol, tocotrienol), vitamin K (phylloquinone, naphthoquinone), vitamin B1 (thiamin), vitamin B2 (riboflavin, vitamin G), vitamin B3 (niacin, nicotinic acid, vitamin PP), vitamin B5 (pantothenic acid), vitamin B6 (pyridoxine, pyridoxal, pyridoxamine), vitamin B7 (biotin, vitamin H), vitamin B9 (folic acid, folate, folacin, vitamin M, pteroyl-L-glutamic acid), vitamin B12 (cobalamin, cyanocobalamin), and vitamin C (ascorbic acid).

Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins.

In some embodiments, the vitamin is a fat-soluble vitamin chosen from vitamin A, D, E, K and combinations thereof. In other embodiments, the vitamin is a water-soluble vitamin chosen from vitamin B 1, vitamin B2, vitamin B3, vitamin B6, vitamin B12, folic acid, biotin, pantothenic acid, vitamin C and combinations thereof.

In particular embodiments of this application, the preservative is chosen from antimicrobials, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone.

According to a particular embodiment, the preservative is a sulfite. Sulfites include, but are not limited to, sulfur dioxide, sodium bisulfite, and potassium hydrogen sulfite.

According to another particular embodiment, the preservative is a propionate. Propionates include, but are not limited to, propionic acid, calcium propionate, and sodium propionate.

According to yet another particular embodiment, the preservative is a benzoate. Benzoates include, but are not limited to, sodium benzoate and benzoic acid.

In another particular embodiment, the preservative is a sorbate. Sorbates include, but are not limited to, potassium sorbate, sodium sorbate, calcium sorbate, and sorbic acid.

In still another particular embodiment, the preservative is a nitrate and/or a nitrite. Nitrates and nitrites include, but are not limited to, sodium nitrate and sodium nitrite.

In yet another particular embodiment, the at least one preservative is a bacteriocin, such as, for example, nisin.

In another particular embodiment, the preservative is ethanol or ozone.

Non-limiting examples of antienzymatics suitable for use as preservatives in particular embodiments of the application include ascorbic acid, citric acid, and metal chelating agents such as ethylenediaminetetraacetic acid (EDTA).

Hydration products help the body to replace fluids that are lost through excretion. For example, fluid is lost as sweat in order to regulate body temperature, as urine in order to excrete waste substances, and as water vapor in order to exchange gases in the lungs. Fluid loss can also occur due to a wide range of external causes, non-limiting examples of which include physical activity, exposure to dry air, diarrhea, vomiting, hyperthermia, shock, blood loss, and hypotension. Diseases causing fluid loss include diabetes, cholera, gastroenteritis, shigellosis, and yellow fever. Forms of malnutrition that cause fluid loss include the excessive consumption of alcohol, electrolyte imbalance, fasting, and rapid weight loss.

In a particular embodiment, the hydration product is a composition that helps the body replace fluids that are lost during exercise. Accordingly, in a particular embodiment, the hydration product is an electrolyte, non-limiting examples of which include sodium, potassium, calcium, magnesium, chloride, phosphate, bicarbonate, and combinations thereof. Suitable electrolytes for use in particular embodiments of this application are also described in U.S. Pat. No. 5,681,569, the disclosure of which is expressly incorporated herein by reference. In particular embodiments, the electrolytes are obtained from their corresponding water-soluble salts. Non-limiting examples of salts for use in particular embodiments include chlorides, carbonates, sulfates, acetates, bicarbonates, citrates, phosphates, hydrogen phosphates, tartrates, sorbates, citrates, benzoates, or combinations thereof. In other embodiments, the electrolytes are provided by juice, fruit extracts, vegetable extracts, tea, or teas extracts.

In particular embodiments of this application, the hydration product is a carbohydrate to supplement energy stores burned by muscles. Suitable carbohydrates for use in particular embodiments of this application are described in U.S. Pat. Nos. 4,312,856, 4,853,237, 5,681,569, and 6,989,171, the disclosures of which are expressly incorporated herein by reference. Non-limiting examples of suitable carbohydrates include monosaccharides, disaccharides, oligosaccharides, complex polysaccharides or combinations thereof. Non-limiting examples of suitable types of monosaccharides for use in particular embodiments include trioses, tetroses, pentoses, hexoses, heptoses, octoses, and nonoses. Non-limiting examples of specific types of suitable monosaccharides include glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, and sialose. Non-limiting examples of suitable disaccharides include sucrose, lactose, and maltose. Non-limiting examples of suitable oligosaccharides include saccharose, maltotriose, and maltodextrin. In other particular embodiments, the carbohydrates are provided by a corn syrup, a beet sugar, a cane sugar, a juice, or a tea.

In another particular embodiment, the hydration agent is a flavanol that provides cellular rehydration. Flavanols are a class of natural substances present in plants, and generally comprise a 2-phenylbenzopyrone molecular skeleton attached to one or more chemical moieties. Non-limiting examples of suitable flavanols for use in particular embodiments of this application include catechin, epicatechin, gallocatechin, epigallocatechin, epicatechin gallate, epigallocatechin 3-gallate, theaflavin, theaflavin 3-gallate, theaflavin 3′-gallate, theaflavin 3,3′ gallate, thearubigin or combinations thereof. Several common sources of flavanols include tea plants, fruits, vegetables, and flowers. In preferred embodiments, the flavanol is extracted from green tea.

In a particular embodiment, the hydration product is a glycerol solution to enhance exercise endurance. The ingestion of a glycerol containing solution has been shown to provide beneficial physiological effects, such as expanded blood volume, lower heart rate, and lower rectal temperature.

Probiotics, in accordance with the teachings of this invention, comprise microorganisms that benefit health when consumed in an effective amount. Desirably, probiotics beneficially affect the human body's naturally-occurring gastrointestinal microflora and impart health benefits apart from nutrition. Probiotics may include, without limitation, bacteria, yeasts, and fungi.

Prebiotics, in accordance with the teachings of this invention, are compositions that promote the growth of beneficial bacteria in the intestines. Prebiotic substances can be consumed by a relevant probiotic, or otherwise assist in keeping the relevant probiotic alive or stimulate its growth. When consumed in an effective amount, prebiotics also beneficially affect the human body's naturally-occurring gastrointestinal microflora and thereby impart health benefits apart from just nutrition. Prebiotic foods enter the colon and serve as substrate for the endogenous bacteria, thereby indirectly providing the host with energy, metabolic substrates, and essential micronutrients. The body's digestion and absorption of prebiotic foods is dependent upon bacterial metabolic activity. which salvages energy for the host from nutrients that escaped digestion and absorption in the small intestine.

According to particular embodiments, the probiotic is a beneficial microorganism that beneficially affects the human body's naturally-occurring gastrointestinal microflora and imparts health benefits apart from nutrition. Examples of probiotics include, but are not limited to, bacteria of the genus Lactobacilli, Bifidobacteria, Streptococci, or combinations thereof, that confer beneficial effects to humans.

In particular embodiments of the invention, the at least one probiotic is chosen from the genus Lactobacilli. Lactobacilli (i.e., bacteria of the genus Lactobacillus, hereinafter “L.”) have been used for several hundred years as a food preservative and for promoting human health. Non-limiting examples of species of Lactobacilli found in the human intestinal tract include L. acidophilus, L. casei. L. fermentum, L. saliva roes, L. brevis, L. leichmaniiii. L. plantarwn. L. cellobios s, L. reuteri, L. rhamnos s, L. GG, L. bulgaricus, and L. thermophilus.

According to other particular embodiments of this invention, the probiotic is chosen from the genus Bifidobacteria. Bifidobacteria also are known to exert a beneficial influence on human health by producing short chain fatty acids (e.g., acetic, propionic, and butyric acids), lactic, and formic acids as a result of carbohydrate metabolism. Non-limiting species of Bifidobacteria found in the human gastrointestinal tract include B. angulatum, B. animalis. B. asteroides. B. bifidum, B. bourn. B. breve, B. catenulatum. B. choerinum, B. coryneforme, B. cuniculi, B. dentium, B. gallicum, B. gallinarum. B indicum, B. longum, B. magnum. B. merycicum, B. minimum, B. pseudocatenulatum, B. pseudolongum. B. psychraerophilum, B. pullorum, B. ruminantium, B. saeculare, B. scardovii, B. simiae. B. subtile, B. thermnnacidophilum, B. thermophilum, B. urinalis, and B. sp.

According to other particular embodiments of this invention, the probiotic is chosen from the genus Streptococcus, Streptococcus thermophilus is a gram-positive facultative anaerobe. It is classified as a lactic acid bacteria and commonly is found in milk and milk products, and is used in the production of yogurt. Other non-limiting probiotic species of this bacteria include Streptococcus salivarus and Streptococcus cremoris.

Prebiotics, in accordance with the embodiments of this invention, include, without limitation, mucopolysaccharides, oligosaccharides, polysaccharides, amino acids, vitamins, nutrient precursors, proteins and combinations thereof.

According to a particular embodiment of this invention, the prebiotic is chosen from dietary fibers, including, without limitation, polysaccharides and oligosaccharides. These compounds have the ability to increase the number of probiotics, which leads to the benefits conferred by the probiotics. Non-limiting examples of oligosaccharides that are categorized as prebiotics in accordance with particular embodiments of this invention include fructooligosaccharides, inulins, isomalto-oligosaccharides, lactilol, lactosucrose, lactulose, pyrodextrins, soy oligosaccharides, transgalacto-oligosaccharides, and xylo-oligosaccharides.

According to other particular embodiments of the invention, the prebiotic is an amino acid. Although a number of known prebiotics break down to provide carbohydrates for probiotics, some probiotics also require amino acids for nourishment.

Prebiotics are found naturally in a variety of foods including, without limitation, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans).

As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrient selected from the group consisting of proteins, carbohydrates, dietary fats, and combinations thereof. Consumption of proteins, carbohydrates, and dietary fats stimulates the release of peptides with appetite-suppressing effects. For example, consumption of proteins and dietary fats stimulates the release of the gut hormone cholecytokinin (CCK), while consumption of carbohydrates and dietary fats stimulates release of Glucagon-like peptide 1 (GLP-1).

Suitable macronutrient weight management agents also include carbohydrates. Carbohydrates generally comprise sugars, starches, cellulose and gums that the body converts into glucose for energy. Carbohydrates often are classified into two categories, digestible carbohydrates (e.g., monosaccharides, disaccharides, and starch) and non-digestible carbohydrates (e.g., dietary fiber). Studies have shown that non-digestible carbohydrates and complex polymeric carbohydrates having reduced absorption and digestibility in the small intestine stimulate physiologic responses that inhibit food intake. Accordingly, the carbohydrates embodied herein desirably comprise non-digestible carbohydrates or carbohydrates with reduced digestibility. Non-limiting examples of such carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates.

In another particular embodiment weight management agent is a dietary fat. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols.

In a particular embodiment, the weight management agents is an herbal extract. Extracts from numerous types of plants have been identified as possessing appetite suppressant properties. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema Sylvestre, Kola Nut, Citrus Auran tium, Yerba Mate, Griffonia Simplicifolia, Guarana, myrrh, guggul Lipid, and black current seed oil.

Another aspect of the present application is directed to an orally consumable composition comprising a sweetener composition of the present application.

“Orally consumable composition,” as used herein, refer to substances which are contacted with the mouth of man or animal, including substances which are taken into and subsequently ejected from the mouth and substances which are drunk, eaten, swallowed or otherwise ingested, and are safe for human or animal consumption when used in a generally acceptable range.

The sweetener compositions noted herein can be used in beverages, broths, and beverage preparations selected from the group comprising carbonated, non-carbonated, frozen, semi-frozen (“slush”), non-frozen, ready-to-drink, concentrated (powdered, frozen, or syrup), dairy, non-dairy, herbal, non-herbal, caffeinated, non-caffeinated, alcoholic, non-alcoholic, flavored, non-flavored, vegetable-based, fruit-based, root/tuber/corm-based, nut-based, other plant-based, cola-based, chocolate-based, meat-based, seafood-based, other animal-based, algae-based, calorie enhanced, calorie-reduced, and calorie-free products, optionally dispensed in open containers, cans, bottles or other packaging. Such beverages and beverage preparations can be in ready-to-drink, ready-to-cook, ready-to-mix, raw, or ingredient form and can use the composition as a sole sweetener or as a co-sweetener.

The sweetener compositions noted herein can be used in foods and food preparations (e.g., sweeteners, soups, sauces, flavorings, spices, oils, fats, and condiments) from dairy-based, cereal-based, baked, vegetable-based, fruit-based, root/tuber/corm-based, nut-based, other plant-based, egg-based, meat-based, seafood-based, other animal-based, algae-based, processed (e.g., spreads), preserved (e.g., meals-ready-to-eat rations), and synthesized (e.g., gels) products.

The sweetener compositions noted herein can be used in candies, confections, desserts, and snacks selected from the group comprising dairy-based, cereal-based, baked, vegetable-based, fruit-based, root/tuber/corm-based, nut-based, gum-based, other plant-based, egg-based, meat-based, seafood-based, other animal-based, algae-based, processed (e.g., spreads), preserved (e.g., meals-ready-to-eat rations), and synthesized (e.g., gels) products. Such candies, confections, desserts, and snacks can be in ready-to-eat, ready-to-cook, ready-to-mix, raw, or ingredient form, and can use the compositions as a sole sweetener or as a co-sweetener.

The sweetener compositions noted herein can be used in prescription and over-the-counter pharmaceuticals, assays, diagnostic kits, and therapies selected from the group comprising weight control, nutritional supplement, vitamins, infant diet, diabetic diet, athlete diet, geriatric diet, low carbohydrate diet, low fat diet, low protein diet, high carbohydrate diet, high fat diet, high protein diet, low calorie diet, non-caloric diet, oral hygiene products (e.g., toothpaste, mouthwash, rinses, floss, toothbrushes, other implements), personal care products (e.g., soaps, shampoos, rinses, lotions, balms, salves, ointments, paper goods, perfumes, lipstick, other cosmetics), professional dentistry products in which taste or smell is a factor (e.g., liquids, chewables, inhalables, injectables, salves, resins, rinses, pads, floss, implements), medical, veterinarian, and surgical products in which taste or smell is a factor (e.g., liquids, chewables, inhalables, injectables, salves, resins, rinses, pads, floss, implements), and pharmaceutical compounding fillers, syrups, capsules, gels, and coating products.

The sweetener compositions noted herein can be used in consumer goods packaging materials and containers selected from the group comprising plastic film, thermoset and thermoplastic resin, gum, foil, paper, bottle, box, ink, paint, adhesive, and packaging coating products.

The sweetener compositions noted herein can be used in goods including sweeteners, co-sweeteners, coated sweetener sticks, frozen confection sticks, medicine spoons (human and veterinary uses), dental instruments, presweetened disposable tableware and utensils, sachets, edible sachets, potpourris, edible potpourris, artificial flowers, edible artificial flowers, clothing, edible clothing, massage oils, and edible massage oils.

Thus the compositions herein are included as suitable sweetener compositions. It should be understood that the singular notations also include plural forms of the abbreviations, e.g., GMG includes GMGs.

The abbreviation “LMWSG” refers to a low molecular weight SG having a molecular weight of equal to or less than 965, e.g., 949, 935, 803, 787, etc.

(1) A LMWSG or mixtures of LMWSGs.

(2) A LMWSG in combination with a stevia extract.

(3) A LMWSG in combination with a sweet tea extract.

(4) A LMWSG in combination with a mogroside extract.

(5) A LMWSG in combination with a stevia glycoside having a molecular weight greater than 965 (daltons).

(6) A LMWSG in combination with a sweet tea component.

(7) A LMWSG in combination with a mogroside component.

(8) A LMWSG in combination with a GSG.

(9) A LMWSG in combination with a GMG.

(10) A LMWSG in combination with a GSG and a GMG.

(11) Any of the above ten combinations further including one or more salts.

(12) Any of the above eleven combinations further including one or more non-SG and non-MG sweeteners.

(13) Any of the above twelve combinations, wherein the resultant sweetener composition reduces, eliminates or masks an undesired taste profile property, such as metallic taste, aftertaste, bitterness, lingering of sweetness, licorice taste, that is associated with an SG or an MG or their constituents in naturally occurring compositions such as extracts.

All stevia glycosides with carboxylic acid group have generally poor solubility. Blending of stevia glycosides, especially stevia glycosides without carboxylic acid groups with a high temperature treatment, such as at temperatures above 20-200 centigrade, preferably 60-90 centigrade, would increase the solubility of stevia glycosides with carboxylic acid group.

The following paragraphs enumerated consecutively from 1 through 201 provide for various aspects of the present invention. In one embodiment, in a first paragraph (1), the present invention provides a composition comprising SGs from Table A, the composition comprising at least two SGs having a molecular weight of equal to or less than 965 daltons (LMWSG).

2. The composition of paragraph 1, wherein the SGs comprise two or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1.

3. The composition of either of paragraphs 1 or 2, comprising two or more of the SGs having a molecular weight equal to or less than 949 daltons.

4. The composition of either of paragraphs 1 or 2, comprising two or more of the SGs having a molecular weight equal to or less than 935 daltons.

5. The composition of either of paragraphs 1 or 2, comprising two or more of the SGs having a molecular weight equal to or less than 803 daltons.

6. The composition of either of paragraphs 1 or 2, comprising two or more of the SGs having a molecular weight equal to or less than 787 daltons.

7. The composition of either of paragraphs 1 or 2, comprising two or more of the SGs having a molecular weight equal to or less than 773 daltons.

8. The composition of either of paragraphs 1 or 2, comprising two or more of the SGs having a molecular weight equal to or less than 675 daltons.

9. The composition of either of paragraphs 1 or 2, comprising two or more of the SGs having a molecular weight equal to or less than 641 daltons.

10. The composition of either of paragraphs 1 or 2, comprising two or more of the SGs having a molecular weight equal to or less than 625 daltons.

11. The composition of either of paragraphs 1 or 2, comprising two or more of the SGs having a molecular weight equal to or less than 611 daltons.

12. The composition of either of paragraphs 1 or 2, comprising two or more of the SGs having a molecular weight equal to or less than 479 daltons.

13. The composition of either of paragraphs 1 or 2, comprising two or more of the SGs having a molecular weight equal to or less than 457 daltons.

14. The composition of paragraph 1, wherein the SGs are steviol-monoside, steviolbioside or rubusoside.

15. The composition of any of paragraphs 1 through 14, wherein the composition is dissolved in a solution.

16. The composition of paragraph 15, wherein the composition is present in a concentration of from about 1 ppm to about 2000 ppm.

17. A composition comprising SGs from Table A, the composition comprising RA from about 50 to about 70% by weight of the total SGs in the composition and one or more SGs having a molecular weight of less than or equal to 965 daltons present in greater than about 10 to about 30% by weight of the total SGs in the composition.

18. The composition of paragraph 17, wherein the SGs comprise one or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1.

19. The composition of either of paragraphs 17 or 18, comprising one or more of the SGs having a molecular weight equal to or less than 949 daltons.

20. The composition of either of paragraphs 17 or 18, comprising one or more of the SGs having a molecular weight equal to or less than 935 daltons.

21. The composition of either of paragraphs 17 or 18, comprising one or more of the SGs having a molecular weight equal to or less than 803 daltons.

22. The composition of either of paragraphs 17 or 18, comprising one or more of the SGs having a molecular weight equal to or less than 787 daltons.

23. The composition of either of paragraphs 17 or 18, comprising one or more of the SGs having a molecular weight equal to or less than 773 daltons.

24. The composition of either of paragraphs 17 or 18, comprising one or more of the SGs having a molecular weight equal to or less than 675 daltons.

25. The composition of either of paragraphs 17 or 18, comprising one or more of the SGs having a molecular weight equal to or less than 641 daltons.

26. The composition of either of paragraphs 17 or 18, comprising one or more of the SGs having a molecular weight equal to or less than 625 daltons.

27. The composition of either of paragraphs 17 or 18, comprising one or more of the SGs having a molecular weight equal to or less than 611 daltons.

28. The composition of either of paragraphs 17 or 18, comprising one or more of the SGs having a molecular weight equal to or less than 479 daltons.

29. The composition of either of paragraphs 17 or 18, comprising one or more of the SGs having a molecular weight equal to or less than 457 daltons.

30. The composition of paragraph 17, wherein the SGs are steviol-monoside, steviolbioside or rubusoside.

31. The composition of any of paragraphs 17 through 30, further comprising from about 5 to about 15% by weight RB of the total SGs in the composition.

32. The composition of any of paragraphs 17 through 31, wherein the composition is dissolved in a solution.

33. The composition of paragraph 32, wherein the composition is present in a concentration of from about 1 ppm to about 2000 ppm, preferably 5-2000 ppm, more preferably 5-1000 ppm, more preferably 5-500 ppm, most preferably 5-200 ppm.

34. A composition comprising SGs from Table A, the composition comprising a stevia extract comprising RA from about 50 to about 70% by weight and from about 5 to about 15% by weight RB of the total SGs in the composition and one or more SGs having a molecular weight of less than or equal to 965 daltons present in greater than 10 to about 30% by weight of the total SGs in the composition.

35. The composition of paragraph 34, wherein the SGs comprise one or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1.

36. The composition of either of paragraphs 34 or 35, comprising one or more of the SGs having a molecular weight equal to or less than 949 daltons.

37. The composition of either of paragraphs 34 or 35, comprising one or more of the SGs having a molecular weight equal to or less than 935 daltons.

38. The composition of either of paragraphs 34 or 35, comprising one or more of the of the SGs having a molecular weight equal to or less than 803 daltons.

39. The composition of either of paragraphs 34 or 35, comprising one or more of the SGs having a molecular weight equal to or less than 787 daltons.

40. The composition of either of paragraphs 34 or 35, comprising one or more of the SGs having a molecular weight equal to or less than 773 daltons.

41. The composition of either of paragraphs 34 or 35, comprising one or more of the SGs having a molecular weight equal to or less than 675 daltons.

42. The composition of either of paragraphs 34 or 35, comprising one or more of the SGs having a molecular weight equal to or less than 641 daltons.

43. The composition of either of paragraphs 34 or 35, comprising one or more of the SGs having a molecular weight equal to or less than 625 daltons.

44. The composition of either of paragraphs 34 or 35, comprising one or more of the SGs having a molecular weight equal to or less than 611 daltons.

45. The composition of either of paragraphs 34 or 35, comprising one or more of the SGs having a molecular weight equal to or less than 479 daltons.

46. The composition of either of paragraphs 34 or 35, comprising one or more of the SGs having a molecular weight equal to or less than 457 daltons.

47. The composition of paragraph 34, wherein the SGs are steviol-monoside, steviolbioside or rubusoside.

48. The composition of any of paragraphs 34 through 47, wherein the composition is dissolved in a solution.

49. The composition of paragraph 48, wherein the composition is present in a concentration of from about 1 ppm to about 2000 ppm, preferably 5-2000 ppm, more preferably 5-1000 ppm, more preferably 5-500 ppm, most preferably 5-200 ppm.

50. A composition comprising a sweet tea extract, a stevia extract, a swingle extract, a glycosylated sweet tea extract, a glycosylated stevia extract, a glycosylated swingle extract, a glycosylated sweet tea glycoside, a glycosylated steviol glycoside, a glycosylated mogroside and mixtures thereof and one or more SGs having a molecular weight of less than or equal to 965 daltons present in greater than about 10 to about 50% by weight of the total SGs in the composition.

51. The composition of paragraph 50, wherein the stevia extract comprises one or more stevia extract components.

52. The composition of paragraph 51, wherein the stevia extract component is one or more of rebaudioside A, rebaudioside B, rebaudioside D, rebaudioside E, rebaudioside M, rebaudioside 0, or mixtures thereof.

53. The composition of paragraph 52, wherein the stevia extract component is rebaudioside A with a purity of 20%, 30%, 40%, 50%, 60%, 80%, 90%, 95%, 97%, 98%, 99% or 100%.

54. The composition of paragraph 52, wherein the stevia extract component is a salt form.

55. The composition of paragraph 50, wherein the swingle extract comprises one or more mogroside extract components.

56. The composition of paragraph 55, wherein the mogroside extract component is one or more of mogroside V, mogroside IV, siamenoside I, 11-oxomogroside V and mixtures thereof.

57. The composition of paragraph 56, wherein the mogroside extract component is a salt form.

58. The composition of paragraph 50, wherein the glycosylated stevia extract comprises glycosylation products of steviol, stevioside, steviolbioside, rebaudioside A, rebaudioside B, rebaudioside C, rebaudioside D, rebaudioside E, rebaudioside F, rebaudioside M, rebaudioside 0, rebaudioside H, rebaudioside I, rebaudioside L, rebaudioside N, rebaudioside K, rebaudioside J, rubusoside, dulcoside A and mixtures thereof.

59. The composition of paragraph 50, wherein the glycosylated steviol glycoside comprises glycosylation products of steviol, stevioside, steviolbioside, rebaudioside A, rebaudioside B, rebaudioside C, rebaudioside D, rebaudioside E, rebaudioside F, rebaudioside M, rebaudioside 0, rebaudioside H, rebaudioside I, rebaudioside L, rebaudioside N, rebaudioside K, rebaudioside J, rubusoside, dulcoside A and mixtures thereof.

60. The composition of paragraph 59, wherein the glycosylated steviol glycoside is a salt form.

61. The composition of paragraph 50, wherein the glycosylated swingle extract comprises a glycosylated mogroside II, a glycosylated mogroside III, a glycosylated mogroside IV, a glycosylated mogroside V, a glycosylated siamenoside I or a glycosylated 11-oxomogroside V and mixtures thereof.

62. The composition of paragraph 50, wherein the glycosylated mogroside comprises a glycosylated mogroside II, a glycosylated mogroside III, a glycosylated mogroside IV, a glycosylated mogroside V, a glycosylated siamenoside I or a glycosylated 11-oxomogroside V and mixtures thereof.

63. The composition of paragraph 62, wherein the glycosylated mogroside is a salt form.

64. The composition of any of paragraphs 1 through 63, further comprising a salt.

65. The composition of paragraph 64, wherein the salt comprises sodium carbonate, sodium bicarbonate, sodium chloride, potassium chloride, magnesium chloride, sodium sulfate, magnesium sulfate, potassium sulfate and mixtures thereof.

66. The composition of any of paragraphs 1 through 65, further comprising a non-SG and non-MG sweetener.

67. The composition of paragraph 66, wherein the sweetener comprises sorbitol, xylitol, mannitol, aspartame, acesulfame-K, neotame, erythritol, trehalose, raffinose, cellobiose, tagatose, DOLCIA PRIMA™ allulose, inulin, N—[N-[3-(3-hydroxy-4-methoxyphenyl)propyl]-alpha-aspartyl]-L-phenylalanine 1-methyl ester, glycyrrhizin, sodium cyclamate, and mixtures thereof.

68. The composition of any of paragraphs 1 through 67, wherein the one or more SGs are low molecular weight SG(s).

69. The composition of paragraph 68, wherein the one or more SG(s) have a molecular weights less than or equal to 787 comprising related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D and mixtures thereof.

70. The composition of any of paragraphs 1 through 67, wherein the one or more SGs include a free carboxyl group comprising steviol-monoside, SG-4, dulcoside A1, iso-steviolbioside, reb-G1, steviolbioside, reb-F1, reb-R1, dulcoside, SG-3, iso-reb B, reb B, reb L1 and mixtures thereof.

71. The composition of paragraph 70, wherein the carboxyl group is present as a salt form.

72. A composition comprising rebaudioside D, rebaudioside M, a mixture of rebaudioside D and rebaudioside M or a mixture of rebaudioside A, rebaudioside D and rebaudioside M and a SG having a molecular weight of equal to or less than 965 daltons (LMWSG).

73. The composition of paragraph 72, wherein the LMWSG has a molecular weight of less than or equal to 787.

74. The composition of either 72 or 73, wherein the LMWSG comprises related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D and mixtures thereof.

75. The composition of any of paragraphs 72 through 74, wherein the LMWSG include a free carboxyl group comprising steviol-monoside, SG-4, dulcoside A1, iso-steviolbioside, reb-G1, steviolbioside, reb-F1, reb-R1, dulcoside, SG-3, iso-reb B, reb B, reb L1 and mixtures thereof.

76. The composition of paragraph 75, wherein the carboxyl group is present as a salt form.

77. The composition of any of paragraphs 1 through 76, wherein a process to prepare a steviol glycoside having a carboxylic acid salt or enzyme-transformed steviol glycoside from a steviol glycoside starting material is utilized, comprising the step:

subjecting a starting material of steviol glycoside to a base or an enzyme to provide a hydrolyzed steviol glycoside having a carboxylic acid salt moiety or enzyme-transformed steviol glycoside as a product mixture, such that the composition further includes the product mixture.

78. The composition of paragraph 77, wherein the steviol glycoside starting material comprises Rebaudioside A, Rebaudioside C, Rebaudioside D, Rebaudioside E, Stevioside, Rubusoside and mixtures thereof.

79. The composition of paragraph 77, wherein the product mixture comprises a steviol glycoside carboxylic acid salt, unhydrolyzed steviol glycoside starting material, base and optionally, caramelized product(s).

80. The composition of paragraph 79, further comprising the step

subjecting the steviol glycoside carboxylic acid salt, the unhydrolyzed steviol glycoside starting material, base and optionally caramelized product(s) to an acid to provide a free carboxylic acid steviol glycoside, unhydrolyzed steviol glycoside starting material, salt and the optionally caramelized product(s) as a reaction mixture, such that the composition further includes the reaction mixture.

81. The composition of any of paragraphs 77 through 80, further comprising the step of separating each component of the reaction mixture to provide one or more purified product(s) from the reaction mixture in the composition.

82. The composition of any of paragraphs 1 through 81, wherein the percentage of each low molecular weight SG in the composition reduces, eliminates or masks lingering.

83. The composition of any of paragraphs 1 through 81, wherein the percentage of each low molecular weight SG in the composition reduces, eliminates or masks aftertaste, metallic taste, bitterness, or licorice taste.

84. The composition of any of paragraphs 1 through 81, wherein the percentage of each low molecular weight SG in the composition provides an improved taste profile compared to an untreated sweet tea extract, an SG extract, an MG extract, a GSG or an GMG composition.

85. The composition of any of paragraphs 1 through 84, wherein the composition is used as a flavor or as a sweetener.

86. An orally consumable composition comprising the composition of any one of paragraphs 1 through 84.

87. The orally consumable composition of paragraph 86, wherein the one or more LMWSG(s) having a molecular weight of less than or equal to 965 daltons constitute at least 1 ppm, 5 ppm, 10 ppm, 20 ppm, 30 ppm, 40 ppm, 50 ppm, 60 ppm, 70 ppm, 80 ppm, 90 ppm, 100 ppm, 150 ppm, 200 ppm, 300 ppm, 400 ppm, 600 ppm, 800 ppm, 1000 ppm, 1200 ppm, 1500 ppm, 2000 ppm or 5000 ppm of the total orally consumable composition.

88. A process to prepare a steviol glycoside having a carboxylic acid salt or an enzyme transformed steviol glycoside from a steviol glycoside starting material, comprising the step:

subjecting a starting material of steviol glycoside to a base or an enzyme to provide a hydrolyzed steviol glycoside having a carboxylic acid salt moiety or an enzyme transformed steviol glycoside as a product mixture.

89. The process of paragraph 88, wherein the steviol glycoside starting material comprises Rebaudioside A, Rebaudioside C, Rebaudioside D, Rebaudioside E, Stevioside, Rubusoside and mixtures thereof.

90. The process of paragraph 88, wherein the product mixture comprises a steviol glycoside carboxylic acid salt, unhydrolyzed steviol glycoside starting material, base and optionally, caramelized product(s).

91. The process of paragraph 90, further comprising the step

subjecting the steviol glycoside carboxylic acid salt, the unhydrolyzed steviol glycoside starting material, base and optionally caramelized product(s) to an acid to provide a free carboxylic acid steviol glycoside, unhydrolyzed steviol glycoside starting material, salt and the optionally caramelized product(s) as a reaction mixture.

92. The process of any of paragraphs 88 through 91, further comprising the step of separating each component of the reaction mixture to provide one or more purified product(s) from the reaction mixture.

93. The process of any of paragraphs 88 through 92, wherein the product mixture, the reaction mixture or the purified products from the product mixture or reaction mixture can be added to any of the compositions of paragraphs 1 through 76.

94. A flavoring or a sweetener composition comprising steviol glycosides wherein low molecular weight steviol glycosides having a molecular weight of less than or equal to 787 are present in solution at 1 ppm, 5 ppm, 10 ppm, 20 ppm, 30 ppm, 40 ppm, 50 ppm, 60 ppm, 70 ppm, 80 ppm, 90 ppm, 100 ppm, 150 ppm, 200 ppm, 300 ppm, 400 ppm, 600 ppm, 800 ppm, 1000 ppm, 1200 ppm, 1500 ppm, 2000 ppm or 5000 ppm.

95. A flavoring or a sweetener composition comprising stevia glycosides consisting of at least one LMWSGs.

96. The flavoring or sweetener composition of paragraph 95, wherein the content of LMWSGs in the composition is from about 0.5% to about 99.5% by weight of the composition.

97. A flavoring or a sweetener composition comprising steviol glycosides and at least one LMWSG.

98. The flavoring or sweetener composition of paragraph 96, wherein the content of LMWSGs in the composition is from about 0.5% to about 99.5% by weight of the composition.

99. A stevia composition comprising one or more steviol glycosides selected from steviol monoside, steviolbioside, rubusoside, dulcoside.

100. The stevia composition according to paragraph 99, wherein the stevia composition could be used as flavor or sweetener enhancer.

101. The stevia composition according to paragraph 100, wherein the stevia composition improves the taste profile including improving mouthfeel and/or reducing lingering.

102. The stevia composition according to any of paragraphs 99 through 101, wherein the concentration of stevia composition is above 5 ppm, 10 ppm, 50 ppm, 100 ppm, 200 ppm, 500 ppm, 1,000 ppm, 2,000 ppm, 5,000 ppm.

103. The stevia composition according to any of paragraphs 99 through 101, wherein the stevia composition is obtained by fermentation, enzymatic processing, or chemical synthesis such as by hydrolysis of stevia glycosides.

104. A composition of steviol glycosides comprising at least two steviol glycosides.

105. The composition of paragraph 104, wherein one of the steviol glycoside is selected from rebaudioside A (RA), rebaudioside B (RB), rebaudioside D (RD) or rebaudioside M (RM) and mixtures thereof.

106. The composition of paragraph 104, wherein one steviol glycoside is selected from LMWSGs and mixtures thereof.

107. The composition of paragraph 1 or paragraph 2, wherein the SGs are derived from sweet tea, stevia leaves, enzymatic conversion, fermentation or chemical synthesis.

108. The composition of paragraph 1 or paragraph 2, wherein the SGs have the following parent structures of formulas Sv or Iso-Sv:

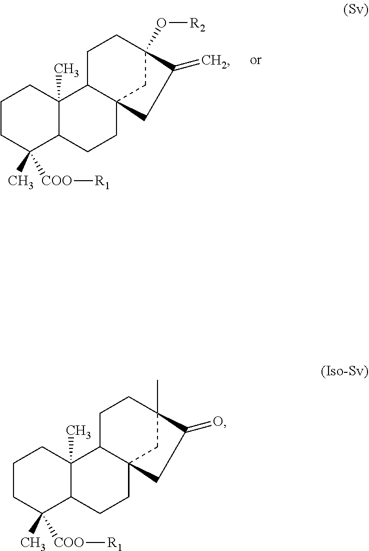

wherein R₁ and R₂ are substituent groups selected from the groups comprising glucosyl (G), rhamnosyl (R), xylosyl (X), deoxy-glucosyl (dG), frucosyl (F), arabinosyl (A), and galactosyl (Ga).

109. The composition of paragraph 108, wherein the SGs have the structures represented by the formulas: SvGₐ, SvR₁Gₚ, SvX₁Gₘ, SvdG₁G_{q}, Iso-SvGᵣ, SvF₁G₃, SvA₁G₄ or SvGa₁G₄, wherein:

the values of n, p, m, q and r are integers, and represent the number of corresponding substituent groups, respectively, and

n is from 1 to 6; p is from 1 to 6; m is from 1 to 5; q is from 2 to 3; and r is from 2 to 4.

110. The composition of any of paragraphs 1, 2, or 108 through 109, comprising one or more of the SGs having a molecular weight equal to or less than 949 daltons.

111. The composition of any of paragraphs 1, 2, or 108 through 109, comprising one or more of the SGs having a molecular weight equal to or less than 935 daltons.

112. The composition of any of paragraphs 1, 2, or 108 through 109, comprising one or more of the SGs having a molecular weight equal to or less than 803 daltons.

113. The composition of any of paragraphs 1, 2, or 108 through 109, comprising one or more of the SGs having a molecular weight equal to or less than 787 daltons.

114. The composition of any of paragraphs 1, 2, or 108 through 109, comprising one or more of the SGs having a molecular weight equal to or less than 773 daltons.

115. The composition of any of paragraphs 1, 2, or 108 through 109, comprising one or more of the SGs having a molecular weight equal to or less than 675 daltons.

116. The composition of any of paragraphs 1, 2, or 108 through 109, comprising one or more of the SGs having a molecular weight equal to or less than 641 daltons.

117. The composition of any of paragraphs 1, 2, or 108 through 109, comprising one or more of the SGs having a molecular weight equal to or less than 625 daltons.

118. The composition of any of paragraphs 1, 2, or 108 through 109, comprising one or more of the SGs having a molecular weight equal to or less than 611 daltons.

119. The composition of any of paragraphs 1, 2, or 108 through 109, comprising one or more of the SGs having a molecular weight equal to or less than 479 daltons.

120. The composition of any of paragraphs 1, 2, or 108 through 109, comprising one or more of the SGs having a molecular weight equal to or less than 457 daltons.

121. The composition of any of paragraphs 1, 2, or 108 through 109, wherein the SGs are steviol-monoside, steviolbioside, rubusoside or dulcoside B.

122. The composition of any of paragraphs 1, 2 or 108 through 121, wherein the composition is dissolved in a solution.

123. The composition of paragraph 122, wherein the composition is present in a concentration of from about 0 ppm, preferably about 1 ppm, preferably about 5 ppm, still preferably about 10 ppm, still preferably about 20 ppm to about 200 ppm, preferably 1000 ppm, preferably about 2000 ppm.

124. The composition of paragraph 121 or paragraph 122, wherein the rubusoside is present in a concentration of less than about 100 ppm.

125. The composition of paragraph 121 or paragraph 122, wherein the steviol-monoside is present in a concentration of less about 65 ppm.

126. The composition of paragraph 121 or paragraph 122, wherein the dulcoside B is present in a concentration of less than about 160 ppm.

127. The composition of paragraph 121 or paragraph 122, wherein the steviolbioside is present in a concentration of less than about 100 ppm.

128. The composition of any of paragraph 1, 2 or 107 through 127, wherein the composition is used as flavor modifier or flavor enhancer.

129. The composition of paragraph 128, wherein the composition is used for enhancing the mouthfeel and decreased lingering.

130. A method for preparing the composition of any of paragraphs 1, 2 or 107 through 121, wherein the method comprises steps of hydrolyzing the stevia extract by adding alkali.

131. A composition comprising two groups of SG, the first group of SG comprises one or more SGs from any of paragraphs 1, 2 or 107 through 109, the second group of SG comprises one or more SGs selected from the group consisting of RA, RB, RD, RM, Stevioside, RC and the combination thereof.

132. The composition of paragraph 131, wherein the ratio of the weight of the first group and the second group of SG is 1:99, 2:98, 3:97, 4:96; 5:95; 6:94; 7:93; 8:94; 9:91; 10:90; 11:89; 12:88; 13:87; 14:86; 15:85; 16:84; 17:83; 18:82; 19:81; 20:80; 21:79; 22:78; 23:77; 24:76; 25:75; 26:74; 27:73; 28:72; 29:71; 30:70; 31:69; 32:68; 33:67; 34:66; 35:65; 36:64; 37:63; 38:62; 39:61; 40:60; 41:59; 42:58; 43:57; 44:56; 45:55; 46:54; 47:53; 48:52; 49:51; 50:50; 51:49; 52:48; 53:47; 54:45; 55:45; 56:44; 57:43; 58:42; 59:41; 60:40; 61:39; 62:38; 63:37; 64:36; 65:35; 66:37; 67:33; 68:32; 69:31; 70:30; 71:29; 72:28; 73:27; 74:26; 75:25; 76:24; 77:23; 78:22; 79:21; 80:20; 81:19; 82:18; 83:17; 84:16; 85:15; 86:14; 87:13; 88:12; 89:11; 90:10; 91:9; 92:8; 93:7; 94:6; 95:5; 96:4; 97:3; 98:2 or 99:1.

133. The composition of paragraph 131 or paragraph 132, wherein the first group of SGs is present in an amount of less than 50 wt %, preferably less than 40 wt %, preferably less than wt 30%, preferably less than 20 wt %, preferably less than 10 wt %, more preferably less than 5 wt %, and even more preferably less than 1 wt % by weight of the composition.

134. A method to reduce the lingering of sweetness in a sweetened composition comprising the step:

providing a sweetener composition having a sweetness lingering time period of X;

adding a low molecular weight steviol glycoside (LMWSG) to the sweetener composition, wherein the sweetness lingering time period of X is reduced by at least 30 percent.

135. The method of paragraph 134, wherein the sweetness lingering time period is reduced by at least 20 percent.

136. The method of paragraph 134, wherein the sweetness lingering time period is reduced by at least 10 percent.

137. The method of any of paragraphs 134 through 136, wherein the LMWSG is selected from the group consisting of steviolmonoside, steviolbioside, dulcoside, rubusoside and mixtures thereof.

138. The method of any of paragraphs 134 through 137, wherein the sweetener composition comprises thaumatin.

139. The composition of paragraph 50, wherein, the one or more SGs having a molecular weight of less than or equal to 965 daltons are present in greater than about 10 to about 30% by weight of the total SGs in the composition.

140. The orally consumable composition of paragraph 86, wherein, the orally consumable is a beverage, a foodstuff, a pharmaceutical composition, an edible gel mix and compositions, a dental composition or a beverage product.

141. The orally consumable composition of paragraph 86, wherein the one or more LMWSG(s) having a molecular weight of less than or equal to 965 daltons constitute from about 1 ppm to about 5000 ppm of the total orally consumable composition.

142. The orally consumable composition of paragraph 141, wherein the one or more LMWSG(s) constitute from about 5 ppm to about 5000 ppm.

143. The orally consumable composition of paragraph 142, wherein the one or more LMWSG(s) constitute from about 5 ppm to about 3000 ppm.

144. The orally consumable composition of paragraph 143, wherein the one or more LMWSG(s) constitute from about 5 ppm to about 1000 ppm.

145. The orally consumable composition of paragraph 144, wherein the one or more LMWSG(s) constitute from about 5 ppm to about 500 ppm.

146. The orally consumable composition of paragraph 145, wherein the one or more LMWSG(s) constitute from about 5 ppm to about 200 ppm.

147. The flavoring or the sweetener composition of paragraph 94, wherein the low molecular weight steviol glycosides are present in solution in a range of from about 5 ppm to about 5000 ppm.

148. The flavoring or the sweetener composition of paragraph 94, wherein the low molecular weight steviol glycosides are present in solution in a range of from about 5 ppm to about 3000 ppm.

149. The flavoring or the sweetener composition of paragraph 94, wherein the low molecular weight steviol glycosides are present in solution in a range of from about 5 ppm to about 1000 ppm.

150. The flavoring or the sweetener composition of paragraph 94, wherein the low molecular weight steviol glycosides are present in solution in a range of from about 5 ppm to about 500 ppm.

151. The flavoring or the sweetener composition of paragraph 94, wherein the low molecular weight steviol glycosides are present in solution in a range of from about 5 ppm to about 200 ppm.

152. The flavoring or the sweetener composition of paragraph 94, wherein the low molecular weight steviol glycosides are present in solution in a range of about 1 ppm, 5 ppm, 10 ppm, 20 ppm, 30 ppm, 40 ppm, 50 ppm, 60 ppm, 70 ppm, 80 ppm, 90 ppm, 100 ppm, 150 ppm, 200 ppm, 300 ppm, 400 ppm, 600 ppm, 800 ppm, 1000 ppm, 1200 ppm, 1500 ppm, 2000 ppm or 5000 ppm.

153. The stevia composition according to any of paragraphs 99 through 101, wherein the concentration of the stevia composition is from about 5 ppm to about 3000 ppm.

154. The stevia composition according to any of paragraphs 99 through 101, wherein the concentration of the stevia composition is from about 5 ppm to about 1000 ppm.

155. The stevia composition according to any of paragraphs 99 through 101, wherein the concentration of the stevia composition is from about 5 ppm to about 500 ppm.

156. The stevia composition according to any of paragraphs 99 through 101, wherein the concentration of the stevia composition is from about 5 ppm to 200 ppm.

157. The stevia composition according to any of paragraphs 99 through 101, wherein the concentration of stevia composition is above 5 ppm, 10 ppm, 50 ppm, 100 ppm, 200 ppm, 500 ppm, 1,000 ppm, 2,000 ppm, or 5,000 ppm.

158. The composition of paragraph 122, wherein the composition is present in a concentration of from about 5 ppm to about 2000 ppm.

159. The composition of paragraph 122, wherein the composition is present in a concentration of from about 5 ppm to about 1000 ppm.

160. The composition of paragraph 122, wherein the composition is present in a concentration of from about 5 ppm to about 500 ppm.

161. The composition of paragraph 122, wherein the composition is present in a concentration of from about 5 ppm to about 200 ppm.

162. The composition of paragraph 122, wherein the composition is present in a concentration of from about 1 ppm, about 5 ppm, about 10 ppm, about 20 ppm, about 200 ppm, about 1000 ppm or about 2000 ppm.

163. The composition of paragraph 131, wherein the ratio of the weight of the first group and the second group of SG is 1:99, 2:98, 3:97, 4:96; 5:95; 6:94; 7:93; 8:94; 9:91; 10:90; 11:89; 12:88; 13:87; 14:86; 15:85; 16:84; 17:83; 18:82; 19:81; 20:80; 21:79; 22:78; 23:77; 24:76; 25:75; 26:74; 27:73; 28:72; 29:71; 30:70; 31:69; 32:68; 33:67; 34:66; 35:65; 36:64; 37:63; 38:62; 39:61; 40:60; 41:59; 42:58; 43:57; 44:56; 45:55; 46:54; 47:53; 48:52; 49:51; 50:50; 51:49; 52:48; 53:47; 54:45; 55:45; 56:44; 57:43; 58:42; 59:41; 60:40; 61:39; 62:38; 63:37; 64:36; 65:35; 66:37; 67:33; 68:32; 69:31; 70:30; 71:29; 72:28; 73:27; 74:26; 75:25; 76:24; 77:23; 78:22; 79:21; 80:20; 81:19; 82:18; 83:17; 84:16; 85:15; 86:14; 87:13; 88:12; 89:11; 90:10; 91:9; 92:8; 93:7; 94:6; 95:5; 96:4; 97:3; 98:2 or 99:1.

164. The composition of any of paragraphs 1 through 85, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

165. The orally consumable compositions of either paragraphs 86 or 87, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N— [N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

166. The process of any of paragraphs 88 through 93, further comprising the inclusion of one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N— [N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

167. The flavoring or sweetener composition of any of paragraphs 94 through 98 further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

168. The stevia composition of any of paragraphs 99 through 103, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N— [N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

169. The composition of any of paragraphs 104 through 133, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

170. The method to reduce the lingering sweetness in a sweetened composition of any of paragraphs 134 through 138, further comprising the inclusion of one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

171. The composition of paragraph 139, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

172. The orally consumable composition of any of paragraphs 140 through 146, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N— [N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

173. The flavoring or sweetener composition of any of paragraphs 147 through 152, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

174. The stevia composition of any of paragraphs 153 through 157, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N— [N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

175. The composition of any of paragraphs 158 through 163, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N— [N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

176. A composition comprising one or more of rubusoside (RU), steviolmonoside (STM) or steviolbioside (STB) with rebaudioside A (RA) and rebaudioside B (RB), wherein the composition has a reduced sweet lingering aspect that is less than that of the RA/RB combination without the RU, STM or STB present.

177. The composition of paragraph 176, wherein the combination of RA to RB is from about 10:90 to about 90:10 by weight.

178. The composition of paragraph 177, wherein the combination of RA to RB is about 75:15 by weight.

179. The composition of paragraph 176, wherein the ratio of the RU, STM or STB is about 1:4 by weight relative to the weight of the RA/RB combination.

180. The composition of paragraph 176, wherein the combination of RA to RB is about 75:15 by weight and the ratio of the RU, STM or STB is about 1:4 by weight of the weight of the RA/RB combination.

181. A composition comprising one or more of rebaudioside A (RA), rebaudioside B (RB) or rebaudioside D (RD) with rubusoside (RU), wherein a sweet lingering aspect of the composition is decreased in comparison to that of the RA, RB or RD without the RU present.

182. The composition of paragraph 181, wherein the ratio of RA, RB, or RD to RU is about 20:1 to about 5:1.

183. The composition of paragraph 182, wherein the ratio of RA, RB or RD to RU is about 9:1 by weight.

184. A composition comprising thaumatin and one or more of rubusoside (RU) or steviolbioside (STB), wherein a sweet lingering aspect of the composition is decreased in comparison to that of the thaumatin without the RU or STB present.

185. The composition of paragraph 184, wherein the ratio of thaumatin to the RU or STB is about 1:10 to about 1:1.

186. The composition of paragraph 185, wherein the ratio of the thaumatin to the RU or STB is about 5:9.

187. A composition comprising rebaudioside D (RD) in combination with one or more of steviolbioside (STB), dulcoside B (DB), steviolmonoside (STM) or rubusoside (RU), wherein a sweet lingering aspect of the composition is decreased in comparison to that of the RD without the STB, DB, STM or RU present.

188. The composition of paragraph 187, wherein the ratio of the RD to STB, DB, STM or RU is from about 20:1 to about 5:1.

189. The composition of paragraph 188, wherein the ratio of the RD to STB, DB, STM or RU is from about 9:1 to about 7.5:2.5.

190. A composition comprising rebaudioside M (RM) in combination with one or more of steviolbioside (STB), dulcoside B (DB), steviolmonoside (STM) or rubusoside (RU), wherein a sweet lingering aspect of the composition is decreased in comparison to that of the RM without the STB, DB, STM or RU present.

191. The composition or paragraph 190, wherein the ratio of the RM to STB, DB, STM or RU is from about 20:1 to about 5:1.

192. The composition of paragraph 191, wherein the ratio of the RM to STB, DB, STM or RU is from about 9:1 to about 7.5:2.5.

193. A composition comprising rebaudioside A (RA) in combination with one or more of steviolbioside (STB), dulcoside B (DB), steviolmonoside (STM) or rubusoside (RU), wherein a sweet lingering aspect of the composition is decreased in comparison to that of the RA without the STB, DB, STM or RU present.

194. The composition of paragraph 93, wherein the ratio of the RA to STB, DB, STM or RU is from about 20:1 to about 5:1.

195. The composition of paragraph 194, wherein the ratio of the RA to STB, DB, STM or RU is from about 9:1 to about 7.5:2.5.

196. A composition comprising neohesperidine dihydrochalcone (NHDC) in combination with a low molecular weight steviol glycoside (LMWSG) having a molecular weight of less than or equal to about 965 daltons, wherein a menthol aspect of the composition is increased in comparison to that of the NHDC without a LMWSG present.

197. The composition of paragraph 196, wherein the ratio of NHDC to the LMWSG is from about 10:1 to about 1:1.

198. The composition of paragraph 197, wherein the ratio of NHDC to the LMWSG is from about 5:1 to about 2:1.

199. The composition of paragraph 196, wherein the LMWSG comprises rebaudioside B (RB) or rubusoside (RU).

200. The composition of paragraph 199, wherein the ratio of the NHDC to RB or RU is from about 10:1 to about 1:1.

201. The composition of paragraph 200, wherein the ratio of the NHDC to RB or RU is from about 5:1 to about 2:1.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

### Example 1

Evaluate the Sweetness Threshold of Steviolmonoside.

Samples:

Steviolmonoside (STM90, available from Sweet Green Fields, the content of STM is 99.51 wt %) was dissolved in deionized water to prepare 6 solutions with different concentrations.

| Sample # | Concentration/ppm |
|---|---|
| 111 | 100 |
| 167 | 125 |
| 985 | 150 |
| 621 | 175 |
| 322 | 200 |
| 102 | 225 |

Test Method:

The test panel included 6 persons. Each person was requested to rank the solutions based on the sweetness level. Each person was also requested to determine and rank the solutions not considered sweet.

The standard used to consider a solution as non-sweet was a control solution of 1.5% sugar.

Test Results:

| | |
|---|---|
| person | Order of sweetness (from low to high) |

| | | | | | | |
|---|---|---|---|---|---|---|
| #1 | 167 | 111 | 985 | 621 | 102 | 322 |
| #2 | 167 | 111 | 985 | 621 | 322 | 102 |
| #3 | 111 | 322 | 621 | 102 | 167 | 985 |
| #4 | 111 | 985 | 167 | 322 | 621 | 102 |
| #5 | 167 | 621 | 102 | 985 | 111 | 322 |
| #6 | 111 | 167 | 985 | 621 | 322 | 102 |

Note:
data in bold represented that the solution was not considered sweet by the evaluation panel member.

Data Analysis

| | Ratio of | Ratio of |
|---|---|---|
| concentration | no sweetness | sweetness |
| 100 | 83.30% | 16.70% |
| 125 | 83.30% | 16.70% |
| 150 | 83.30% | 16.70% |
| 175 | 33.30% | 66.70% |
| 200 | 33.30% | 66.70% |
| 225 | 16.70% | 83.30% |

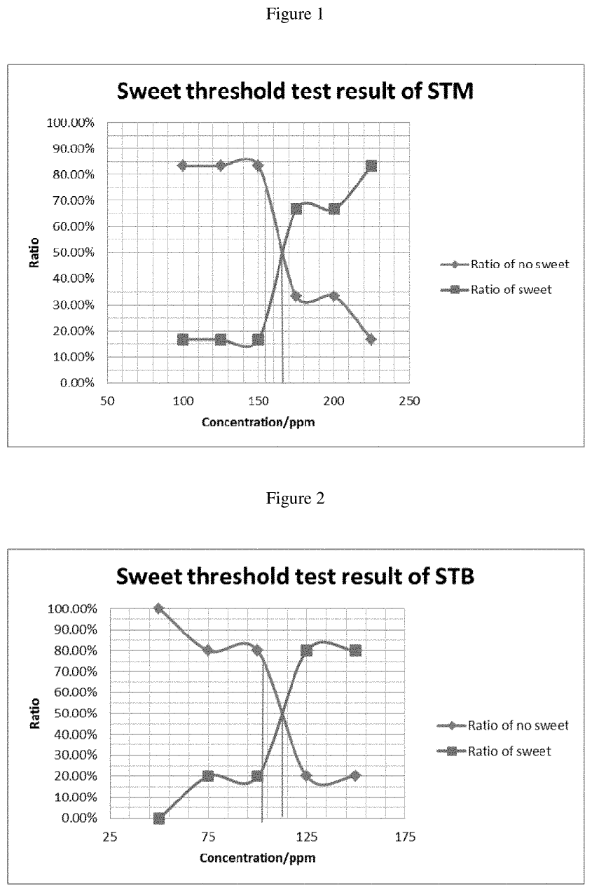

FIG. 1 depicts the concentration at which 50% of panel did not determine that the solution was sweet was about 165 ppm. The concentration at which 75% of panel couldn't taste sweetness was about 155 ppm. According to the results, the average use level is 155 ppm and the average maximum use level is 165 ppm. Therefore, the threshold above about 160 ppm to about 165 ppm results in a sweet tasting solution.

### Example 2

Evaluate the Sweetness Threshold of Steviolbioside.

Steviolbioside (STB90, available from Sweet Green Fields, the content of STB is 90.05 wt %) was dissolved in deionized water to prepare 5 solutions with different concentrations.

| Sample # | Concentration/ppm |
|---|---|
| 102 | 50 |
| 621 | 75 |
| 322 | 100 |
| 111 | 125 |
| 985 | 150 |

Test Method:

The test panel included 5 persons. Each person was requested to rank the solutions based on the sweetness level. Each person was also requested to determine and rank the solutions not considered sweet.

The standard used to consider a solution as non-sweet was a control solution of 1.5% sugar.

Test Results:

| person | Order of sweetness (from low to high) | | | | |
|---|---|---|---|---|---|
| #1 | 621 | 102 | 322 | 111 | 985 |
| #2 | 322 | 102 | 985 | 621 | 111 |
| #3 | 102 | 322 | 621 | 111 | 985 |
| #4 | 102 | 621 | 111 | 322 | 985 |
| #5 | 102 | 621 | 322 | 111 | 985 |

Note: data in bold represented that the solution was not considered sweet by the evaluation panel member.

Data Analysis

| | Ratio of | Ratio of |
|---|---|---|
| concentration | no sweetness | sweetness |
| 50 | 100.00% | 0.00% |
| 75 | 80.00% | 20.00% |
| 100 | 80.00% | 20.00% |
| 125 | 20.00% | 80.00% |
| 150 | 20.00% | 80.00% |

FIG. 2 depicts the concentration at which 50% of panel did not determine that the solution was sweet was about 110 ppm. The concentration at which 75% of panel couldn't taste sweetness was about 100 ppm. According to the results, the average use level is 100 ppm and the average maximum use level is 110 ppm. Therefore, the threshold above about 105 ppm to about 110 ppm results in a sweet tasting solution.

### Example 3

Evaluate the Sweetness Threshold of Rubusoside.

Rubusoside (RU90, available from LAYN, China, the content of RU is 92.57 wt %) was dissolved in deionized water to prepare 5 solutions with different concentrations.

| Sample # | Concentration/ppm |
|---|---|
| 201 | 50 |
| 173 | 75 |
| 164 | 100 |
| 312 | 125 |
| 230 | 150 |

Test Method:

The test panel included 5 persons. Each person was requested to rank the solutions based on the sweetness level. Each person was also requested to determine and rank the solutions not considered sweet.

The standard used to consider a solution as non-sweet was a control solution of 1.5% sugar.

Test Results:

| person | Order of sweetness (from low to high) | | | | |
|---|---|---|---|---|---|
| #1 | 173 | 201 | 164 | 312 | 230 |
| #2 | 164 | 173 | 312 | 201 | 230 |
| #3 | 201 | 173 | 164 | 312 | 230 |
| #4 | 201 | 230 | 173 | 312 | 164 |
| #5 | 201 | 173 | 164 | 312 | 230 |

Note: data in bold represented that the solution was not considered sweet by the evaluation panel member.

Data Analysis

| | Ratio of | Ratio of |
|---|---|---|
| concentration | no sweetness | sweetness |
| 50 | 80.00% | 20.00% |
| 75 | 100.00% | 0.00% |
| 100 | 60.00% | 40.00% |
| 125 | 20.00% | 80.00% |
| 150 | 20.00% | 80.00% |

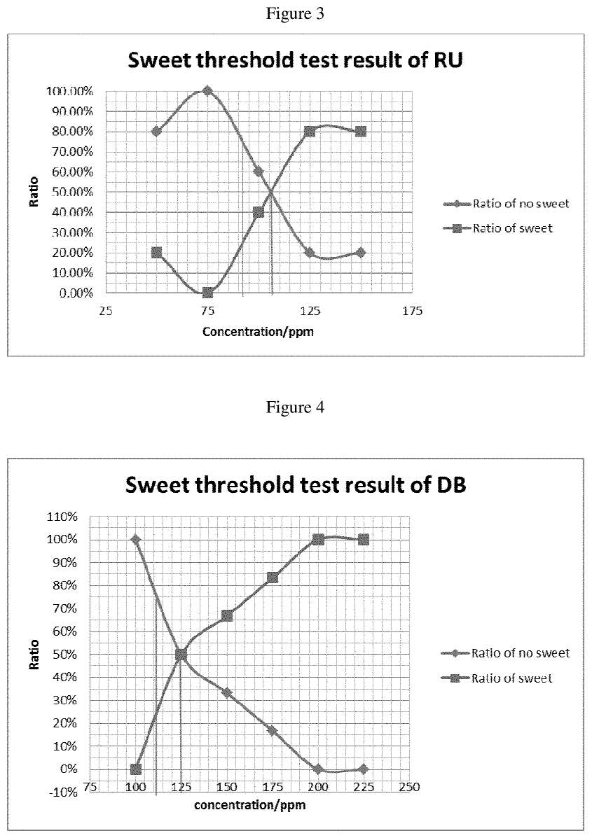

FIG. 3 depicts the concentration at which 50% of panel did not determine that the solution was sweet was about 105 ppm. The concentration at which 75% of panel couldn't taste sweetness was about 90 ppm. According to the results, the average use level is 90 ppm and the average maximum use level is 105 ppm. Therefore, the threshold above about 100 ppm to about 105 ppm results in a sweet tasting solution.

### Example 4

The Taste Improvement of Rubusoside (RU), Steviolbioside (STB) and Steviolmonoside (STM) to stevia Extract.

Materials: Stevia extract RA75/RB15, Steviolbioside (90%, STB90) and Steviolmonoside (90%, STM90) are available from Sweet Green Fields. Rubusoside (90%, RU90) is available from LAYN, China. Stevia extract RA75/RB15 contains 77.72 wt % RA, 16.78% wt RB, and 95.5 wt % TSG(9SG).

Test Method:

The samples were dissolved in deionized water with ultrasound at room temperature and left to sit for 30 min. The concentrations of the solutions were as follow.

| | concentration | | | |
|---|---|---|---|---|
| Solution # | RA75/RB15 | RU90 | STB90 | STM90 |
| 001 | 500 ppm | — | — | — |
| 002 | 400 ppm | 100 ppm | — | — |
| 003 | 400 ppm | — | 100 ppm | — |
| 004 | 400 ppm | — | — | 100 ppm |

Panel: 4 Persons

Method: For evaluation of taste profile, the samples were tested by panel. They were asked to describe the taste profile and score 0-5 according to increasing sugar likeness, bitterness, aftertaste and lingering taste profiles. The results were recorded as the mean value of the results provided by the panel.

| | | Sugar | | |
|---|---|---|---|---|
| Solution # | Taste profile description | like | Bitterness | lingering |
| 001 | Sugar-like front taste; flat; | 4 | 0 | 3 |
| | sweet lingering | | | |
| 002 | full body; bitter taste and | 3.5 | 1.5 | 2 |
| | aftertaste; shorter sweet | | | |
| | lingering than 001 | | | |
| 003 | flat; shorter sweet lingering | 4 | 0.5 | 2.5 |
| | than 001 | | | |
| 004 | full body; more sugar-like | 4.5 | 0 | 1.5 |
| | than 001; shorter sweet | | | |
| | lingering than 001 | | | |

Rubusoside (RU), Steviolbioside (STB) and Steviolmonoside (STM) all improved the taste of RA75/RB15, especially giving an effect of reducing sweet lingering.

Evaluate the Sweet Threshold of Dulcoside B.

Samples:

Dulcoside B (DB90, available from Sweet Green Fields, the content of DB is 93.60 wt %) was dissolved in deionized water to prepare 6 solutions with different concentration.

| Sample # | Concentration/ppm |
|---|---|
| 125 | 100 |
| 167 | 125 |
| 562 | 150 |
| 260 | 175 |
| 473 | 200 |
| 321 | 225 |

Test Method:

The test panel included 6 persons. Each person was requested to rank the solutions based on the sweetness level. Each person was also requested to determine and rank the solutions not considered sweet.

The standard used to consider a solution as non-sweet was a control solution of 1.5% sugar.

Test Results:

| | |
|---|---|
| person | Order of sweetness (from low to high) |

| | | | | | | |
|---|---|---|---|---|---|---|
| #1 | 125 | 562 | 167 | 260 | 473 | 321 |
| #2 | 167 | 125 | 260 | 562 | 473 | 321 |
| #3 | 125 | 167 | 562 | 260 | 321 | 473 |
| #4 | 125 | 167 | 562 | 260 | 473 | 321 |
| #5 | 125 | 167 | 562 | 260 | 473 | 321 |
| #6 | 167 | 125 | 562 | 473 | 260 | 321 |

Note:
data in bold represented that the solution was not considered sweet by the evaluation panel member.

Data Analysis

| | Ratio of | Ratio of |
|---|---|---|
| concentration | no sweet | sweet |
| 100 | 100% | 0 |
| 125 | 50% | 50% |
| 150 | 33.30% | 66.70% |
| 175 | 16.70% | 83.30% |
| 200 | 0 | 100% |
| 225 | 0 | 100% |

FIG. 4 depicts the concentration at which 50% of panel did not determine that the solution was sweet was about 125 ppm. The concentration at which 75% of panel couldn't taste sweetness was about 110 ppm. According to the results, the average use level is 110 ppm and the average maximum use level is 125 ppm. Therefore, the threshold above about 120 ppm to about 125 ppm results in a sweet tasting solution.

The taste improvement of Dulcoside B (DB) to stevia extract.

Materials:

Stevia extract RA75/RB15 and Dulcoside B (90%, DB90) are available from Sweet Green Fields.

Test Method:

The samples were dissolved in deionized water with ultrasound at room temperature and left to sit for 30 min. The concentrations of the solutions were as follow.

Concentration

| Solution # | RA75/RB15 | Dulcoside B 90 |
|---|---|---|
| 001 | 500 ppm | — |
| 002 | 400 ppm | 100 ppm |

Panel: 4 Persons

Method: For evaluation of taste profile, the samples were tested by panel. They were asked to describe the taste profile and score 0-5 according to increasing sugar likeness, bitterness, aftertaste and lingering taste profiles. The results were recorded as the mean value of the results provided by the panel.

Results:

| | | Sugar | | |
|---|---|---|---|---|
| Solution # | Taste profile description | like | Bitterness | lingering |
| 001 | Sugar-like front taste; | 4 | 0 | 3 |
| | flat; sweet lingering | | | |
| 002 | full body; more sugar-like | 4.5 | 0 | 2 |
| | mouth feel than 001; shorter | | | |
| | sweet lingering than 001 | | | |

Dulcoside B (DB) can improve the taste of RA75/RB15, especially giving an effect of reducing the sweet lingering taste.

Method to Produce Rubusoside

Air-dried leaves of Rubus suavissimus S. Lee were extracted with distilled water at approximately 1:15 w/v ratio for 2 hours at 40-45° C. The liquid extract was separated from the solids by centrifugation. The filtered supernatant liquid extract was concentrated and subsequently spray-dried to a powder and designated as the crude extract. The crude extract was dissolved to 80% ethanol aqueous solution at 1:4 w/v. The solution was then heated to 75-80° C. and stirred for 1 hour. The solution was allowed to stand for an hour at 20-25° C. Supernatant and precipitant were separated through centrifugation. The resulted precipitant was blended with 90% ethanol aqueous solution at 1:3 w/v. The resultant mixture was stirred for 30 minutes at room temperature. Supernatant and precipitant were separated through centrifugation. The resulting precipitate was dried in a hot air oven at 60° C. for 8 hours to provide a white powder containing rubusoside which content is about 85-90%.

Method to Produce Steviolmonoside

10 g Rubusoside, 100 mL potassium hydroxide and 100 mL methanol were blended and then refluxed for 1.5 hours. After the reaction mixture gradually returned to room temperature, the pH of the mixture was adjusted to a pH of 2.5 with an aqueous HCl solution (10%). With the addition of HCl, a white solid precipitated. The mixture was stirred continuously for 1 hour in order to complete precipitation. When no additional solid precipitated, the reaction mixture was separated through centrifugation and the resultant precipitate was washed with distilled water. The solid was dried in a hot air oven at 60° C. for 8 hours and a white powder (Steviolmonoside) was obtained.

Method to Produce Steviolbioside

10 g stevioside, 100 mL potassium hydroxide and 100 mL methanol were blended and then refluxed for 1.5 hours. After the reaction mixture gradually returned to room temperature, the pH of the mixture was adjusted to a pH of 2.5 with an aqueous HCl solution (10%). With the addition of HCl, a white solid precipitated. The mixture was stirred continuously for 1 hour in order to complete precipitation. When no additional solid precipitated, the reaction mixture was separated through centrifugation and the resultant precipitate was washed with distilled water. The solid was dried in a hot air oven at 60° C. for 8 hours and a white powder (Steviolbioside) was obtained.

Method to Produce Dulcoside B

10 g Rebaudioside C, 100 mL potassium hydroxide and 100 mL methanol were blended and then refluxed for 1.5 hours. After the reaction mixture gradually returned to room temperature, the pH of the mixture was adjusted to a pH of 2.5 with an aqueous HCl solution (10%). With the addition of HCl, a white solid precipitated. The mixture was stirred continuously for 1 hour in order to complete precipitation. When no additional solid precipitated, the reaction mixture was separated through centrifugation and the resultant precipitate was washed with distilled water. The solid was dried in a hot air oven at 60° C. for 8 hours and a white powder (Dulcoside B) was obtained.

Concentrations of steviol glycosides to reach the sweetness of a 3% sucrose solution, 5% sucrose solution and a 7% sucrose solution

Experimental:

Different solutions of steviol glycosides were prepared. The steviol glycoside solutions were compared with 3%, 5% and 7% sucrose solutions. The target was to determine the amount required to reach the same maximum sweetness as the reference solutions.

Results:

TABLE I

| Steviol | sucrose | |
|---|---|---|
| glycosides | solution | result [ppm] |
| REB-A | 3% | 74 |
| REB-B | 3% | 120 |
| REB-D | 3% | 91 |
| Stevioside | 3% | 160 |
| Rubusoside | 3% | —* |
| REB-A | 5% | 200 |
| REB-B | 5% | 305 |
| REB-D | 5% | 210 |
| Stevioside | 5% | —* |
| Rubusoside | 5% | —* |
| REB-A | 7% | 260 |
| REB-B | 7% | 493 |
| REB-D | 7% | 309 |
| Stevioside | 7% | —* |
| Rubusoside | 7% | —* |

*Denotes that sweetness could not be achieved due to poor taste (bitterness)

Sweetness profile of REB-A, REB-B, REB-D, Stevioside, Rubusoside in different concentrations. Evaluation of onset, time to maximum sweetness, lingering and no taste.

FIG. 5 depicts an example for sweetness profile.

Experimental:

Each person of the test panel drank different steviol glycoside—solutions with defined concentrations (see Table I). During the test, all persons had a time clock. They had to note the appearance-time for five specific points of a sweetness profile (onset, maximum sweetness, lingering on, lingering off and no taste). The results were recorded in following chart:

| | | | | |
|---|---|---|---|---|
| | | LINGERING | LINGERING | NO |
| ONSET | MAX | ON | OFF | TASTE |
| [sec] | [sec] | [sec] | [sec] | [sec] |

REB-A
REB-B
REB-D
Stevioside
Rubusoside

Results

A mean value of all test person results was determined.

Sweetness profile of Rebaudioside A (REB A)

| | | | | | LINGERING | LINGERING | NO |
|---|---|---|---|---|---|---|---|
| | SE | | ONSET | MAX | ON | OFF | TASTE |
| | (%) | | [sec] | [sec] | [sec] | [sec] | [sec] |
| REB A | 3% | person 1 | 2 | 6 | 10 | 15 | 17 |
| REB A | 3% | person 2 | 1.3 | 7 | 9 | 19 | 45 |
| REB A | 3% | person 3 | 1.5 | 10 | 15 | 20 | 55 |
| REB A | 3% | person 4 | 11 | 18 | 31 | 44 | 50 |
| | | mean value | 4.0 | 10.3 | 16.3 | 24.5 | 41.8 |
| REB A | 5% | person 1 | 1 | 3 | 7 | 18 | 40 |
| REB A | 5% | person 2 | 1.3 | 7 | 14 | 20 | 80 |
| REB A | 5% | person 3 | 1 | 15 | 22 | 35 | 80 |
| REB A | 5% | person 4 | 5 | 17 | 30 | 49 | 78 |
| | | mean value | 2.1 | 10.5 | 18.3 | 30.5 | 69.5 |
| REB A | 7% | person 1 | 1 | 5 | 9 | 17 | 28 |
| REB A | 7% | person 2 | 1 | 8 | 15 | 25 | 86 |
| REB A | 7% | person 3 | 0.5 | 8 | 18 | 45 | 100 |
| REB A | 7% | person 4 | 3 | 11 | 18 | 42 | 104 |
| | | mean value | 1.4 | 8 | 15 | 32.3 | 79.5 |

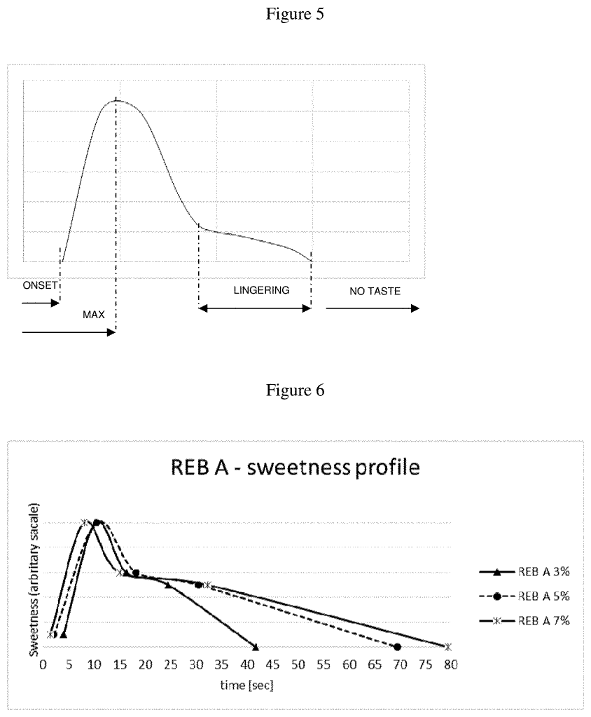

FIG. 6 depicts the sweetness profile for rebaudioside A (REB A) at concentrations of 3%, 5% and 7% in water based on the test data above.

Sweetness profile Rebaudioside B (REB B)

| | | | | | LINGERING | LINGERING | NO |
|---|---|---|---|---|---|---|---|
| | SE | | ONSET | MAX | ON | OFF | TASTE |
| | (%) | | [sec] | [sec] | [sec] | [sec] | [sec] |
| REB B | 3% | person 1 | 2 | 5 | 10 | 13 | 30 |
| REB B | 3% | person 2 | 2 | 6 | 10 | 16 | 34 |
| REB B | 3% | person 3 | 1.5 | 9 | 16 | 25 | 45 |
| | | mean value | 1.8 | 6.7 | 12 | 18 | 36.3 |
| REB B | 5% | person 1 | 1 | 5 | 8 | 16 | 37 |
| REB B | 5% | person 2 | 1 | 9 | 14 | 21 | 58 |
| REB B | 5% | person 3 | 1 | 9 | 15 | 30 | 35 |
| | | mean value | 1 | 7.7 | 12.3 | 22.3 | 43.3 |
| REB B | 7% | person 1 | 0.5 | 4 | 9 | 19 | 35 |
| REB B | 7% | person 2 | 1 | 9 | 16 | 36 | 80 |
| REB B | 7% | person 3 | 1 | 12 | 20 | 30 | 65 |
| | | mean value | 0.8 | 8.3 | 15 | 28.3 | 60 |

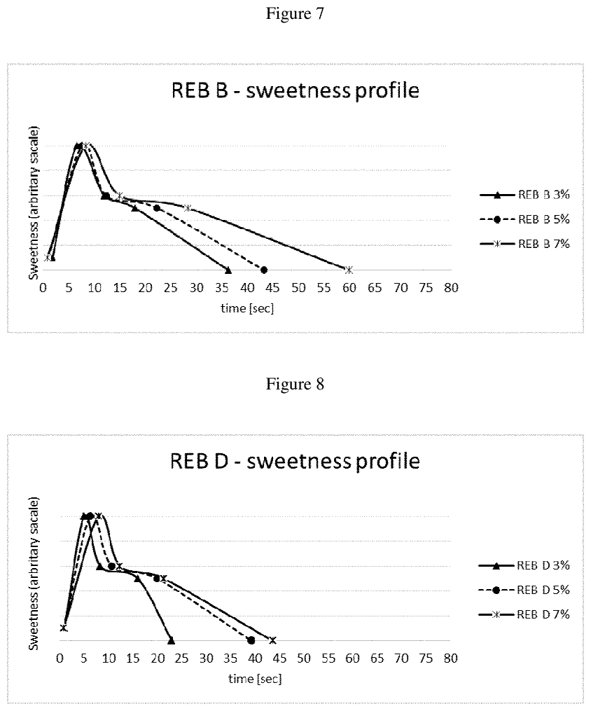

FIG. 7 depicts the sweetness profile for rebaudioside B (REB B) at concentrations of 3%, 5% and 7% in water based on the test data above.

Sweetness profile Rebaudioside D (REB D).

| | | | | | LINGERING | LINGERING | NO |
|---|---|---|---|---|---|---|---|
| | SE | | ONSET | MAX | ON | OFF | TASTE |
| | % | | [sec] | [sec] | [sec] | [sec] | [sec] |
| REB D | 3% | person 1 | 1 | 5 | 9 | 17 | 28 |
| REB D | 3% | person 2 | 1.5 | 5 | 8 | 16 | 21 |
| REB D | 3% | person 3 | 0.5 | 5 | 8 | 15 | 20 |
| | | mean value | 1 | 5 | 8.3 | 16 | 23 |
| REB D | 5% | person 1 | 1 | 6 | 10 | 21 | 47 |
| REB D | 5% | person 2 | 1 | 5 | 10 | 19 | 41 |
| REB D | 5% | person 3 | 0.5 | 8 | 12 | 20 | 30 |
| | | mean value | 0.8 | 6.3 | 10.7 | 20 | 39.3 |
| REB D | 7% | person 1 | 1 | 6 | 10 | 22 | 45 |
| REB D | 7% | person 2 | 1 | 6 | 10 | 16 | 48 |
| REB D | 7% | person 3 | 0.5 | 12 | 17 | 26 | 38 |
| | | mean value | 0.8 | 8 | 12.3 | 21.3 | 43.7 |

FIG. 8 depicts the sweetness profile for rebaudioside D (REB D) at concentrations of 3%, 5% and 7% in water based on the test data above.

Sweetness profile rubusoside (RUB)

| | | | | | LINGERING | LINGERING | NO |
|---|---|---|---|---|---|---|---|
| | SE | | ONSET | MAX | ON | OFF | TASTE |
| | % | | [sec] | [sec] | [sec] | [sec] | [sec] |
| RUB | 3% | person 1 | 0.5 | 4 | 4 | 12 | 21 |
| RUB | 3% | person 2 | 0.5 | 4 | 5 | 10 | 22 |
| RUB | 3% | person 3 | 0.5 | 4 | 6 | 9 | 15 |
| | | mean value | 1 | 4 | 5.0 | 10 | 19 |

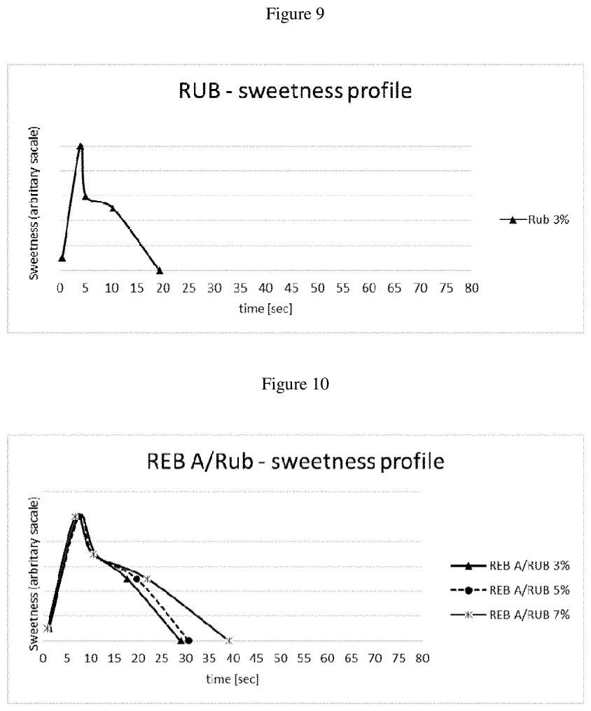

FIG. 9 depicts the sweetness profile for rubusoside (RUB) at a concentration of 3% in water based on the test data above.

Sweetness Profile of Mixtures

Reb-A was mixed with rubusoside to achieve 3, 5 or 7% SE.

The ratio of Reb-A to rubusoside was 9:1 (i.e. for 5% it was 180 ppm to 20 ppm).

FIG. 10 depicts the sweetness profile for rebaudioside A (Reb-A) with rubusoside (Rub) at concentrations of 3%, 5% and 7% in water.

Reb-B was mixed with rubusoside to achieve 3, 5 or 7% SE.

The ratio of Reb-B to rubusoside was 9:1 (i.e. for 5% it was 275 ppm to 27 ppm).

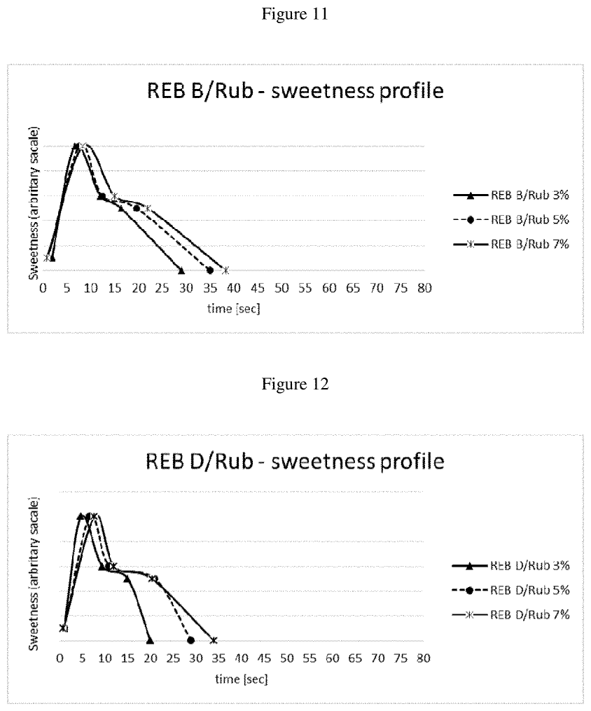

FIG. 11 depicts the sweetness profile for rebaudioside B (Reb-B) with rubusoside (Rub) at concentrations of 3%, 5% and 7% in water.

Reb-D was mixed with rubusoside to achieve 3, 5 or 7% SE.

The ratio of Reb-D to rubusoside was 9:1 (i.e. for 5% it was 189 ppm to 21 ppm).

FIG. 12 depicts the sweetness profile for rebaudioside D (Reb-D) with rubusoside (Rub) at concentrations of 3%, 5% and 7% in water.

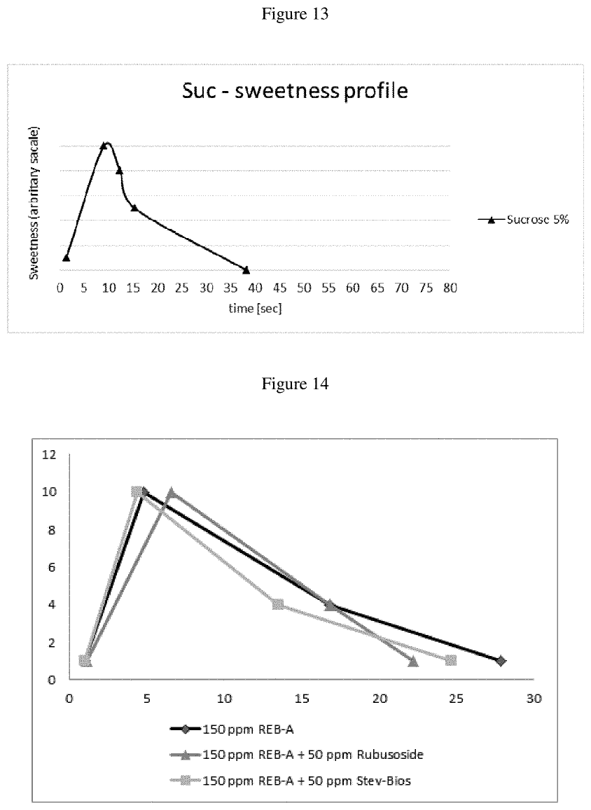

FIG. 13 depicts the sweetness profile of sucrose (Suc) at a concentration of 5% in water.

Sweetness profiles (tabulated mean values)

| | | Time in seconds to | | | | |
|---|---|---|---|---|---|---|
| | SE | | | LINGERING | LINGERING | NO |
| | (%) | ONSET | MAX | ON | OFF | TASTE |
| REB-A | 3% | 4.0 | 10.3 | 16.3 | 24.5 | 41.8 |
| REB | 3% | 1.3 | 7.3 | 11.0 | 17.8 | 29.0 |
| A/RUB | | | | | | |
| REB B | 3% | 1.8 | 6.7 | 12.0 | 18.0 | 36.3 |
| REB | 3% | 1.8 | 6.2 | 11.0 | 16.3 | 29.0 |
| B/RUB | | | | | | |
| REB D | 3% | 1.0 | 5.0 | 8.3 | 16.0 | 23.0 |
| REB | 3% | 1.0 | 4.7 | 9.3 | 15.0 | 20.0 |
| D/RUB | | | | | | |
| Sucrose | 5% | 1.3 | 9.0 | 12.3 | 15.3 | 38.3 |
| REB-A | 5% | 2.1 | 10.5 | 18.3 | 30.5 | 69.5 |
| REB | 5% | 1.1 | 7.5 | 10.5 | 19.8 | 30.8 |
| A/RUB | | | | | | |
| REB B | 5% | 1.0 | 7.7 | 12.3 | 22.3 | 43.3 |
| REB | 5% | 1.0 | 7.8 | 12.1 | 19.7 | 35.0 |
| B/RUB | | | | | | |
| REB D | 5% | 0.8 | 6.3 | 10.7 | 20.0 | 39.3 |
| REB | 5% | 1.0 | 6.7 | 11.0 | 21.0 | 29.0 |
| D/RUB | | | | | | |
| REB-A | 7% | 1.4 | 8.0 | 15.0 | 32.3 | 79.5 |
| REB | 7% | 0.9 | 6.8 | 10.5 | 22.0 | 39.3 |
| A/RUB | | | | | | |
| REB B | 7% | 0.8 | 8.3 | 15.0 | 28.3 | 60.0 |
| REB | 7% | 0.8 | 8.1 | 14.6 | 22.0 | 38.3 |
| B/RUB | | | | | | |
| REB D | 7% | 0.8 | 8.0 | 12.3 | 21.3 | 43.7 |
| REB | 7% | 0.8 | 7.7 | 12.0 | 20.3 | 34.0 |
| D/RUB | | | | | | |

Rubusoside shortens the lingering time and the time to No Taste in mixtures of RA, RB and RD with rubusoside. That makes the samples with rubusoside more similar to sugar in the sweetness profiles.

Test of flavor modifying properties of LMWSGs

150 mg Reb-A were dissolved in 1 liter water. To 200 ml of this solution was added 10 mg rubusoside or 10 mg steviolbioside.

Sensory tests were performed to evaluate the influence of rubusoside and steviolbioside on the sweetness intensity/time profile.

Each person of the test panel drank 10 ml of the corresponding solutions. During the test, all persons had a time clock. They had to note the appearance-time for five specific points of a sweetness profile (onset, maximum sweetness, lingering on, lingering off and no taste). The results were recorded in following table, mean values were calculated from at least 5 individual test persons.

TABLE
Sweetness-Intensity/Time profile of steviol-glycoside
solutions.

| | ONSET | | LINGERING | LINGERING | NO |
|---|---|---|---|---|---|
| | [sec] | MAX | ON | OFF | TASTE |
| REB-A | 1.0 | 4.8 | 16.8 | 27.9 | 52.4 |
| REB- | 1.1 | 6.6 | 16.8 | 22.2 | 47.9 |
| A/RUB | | | | | |
| REB- | 1.0 | 4.4 | 13.5 | 24.7 | 46.4 |
| A/STVB | | | | | |

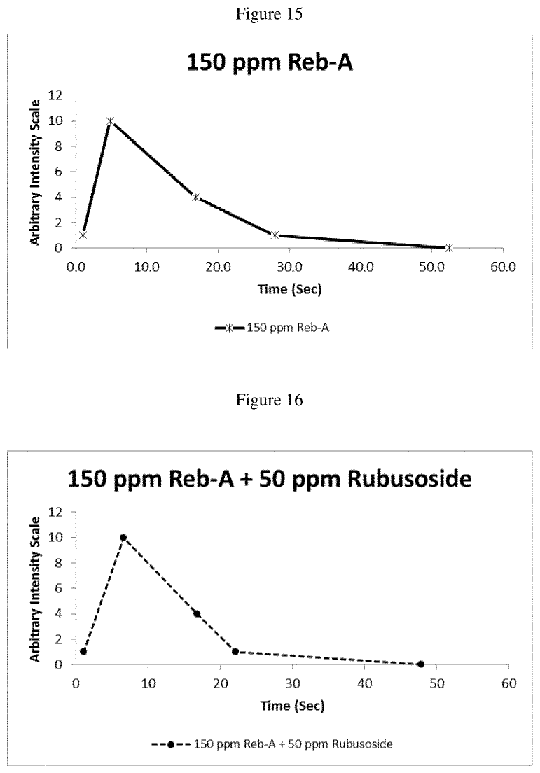

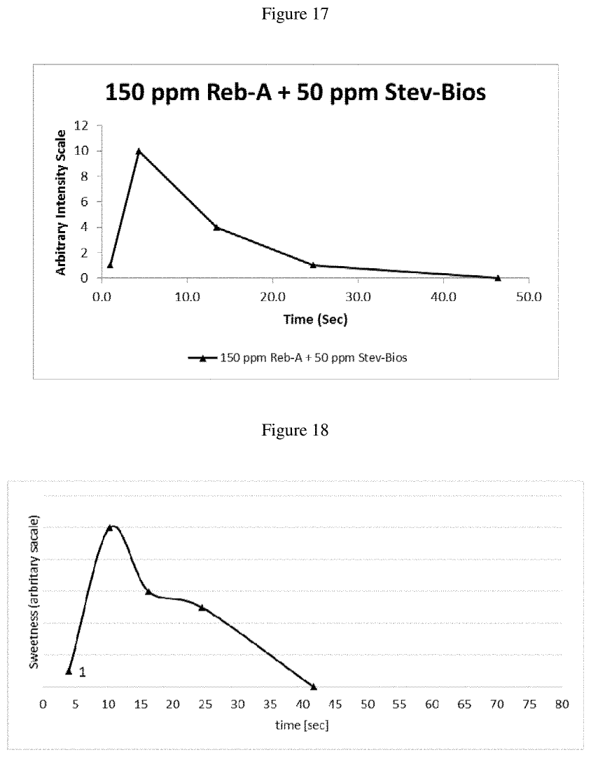

FIGS. 14 through 17 depict the onset/max/lingering/no taste profiles of the above identified solutions.

Sweetness Profile of Thaumatin with/without Rubusoside or Steviolbioside

Each member of the test panel drank the different samples. During the test, all members had a time clock. They had to note the appearance-time for five specific points of a sweetness profile (For example see FIG. 18): onset of sweetness; maximum sweetness; lingering sweetness (no decay of sweetness); lingering off (decay of sweetness, ceiling out phase); and no sweet taste.

For all tests, a minimum of 5 tasters participated. The results obtained are the combined opinion of all 5 tasters. All tests were introduced with an open training session with 50 ppm thaumatin (EPCalin 45). In between the test series, tasters had a break of 30-45 minutes before the next sample.

Thaumatin (EPCalin 45% #20180201) was tested at a concentration of 50 ppm (representing a Sugar Equivalence of 4.5 SE).

Rubusoside (≥90% content) and Steviolbioside (≥90% content) were added in concentrations of 90 ppm (for both proven at a concentration well below 1.5 SE, hence application is flavor modifying).

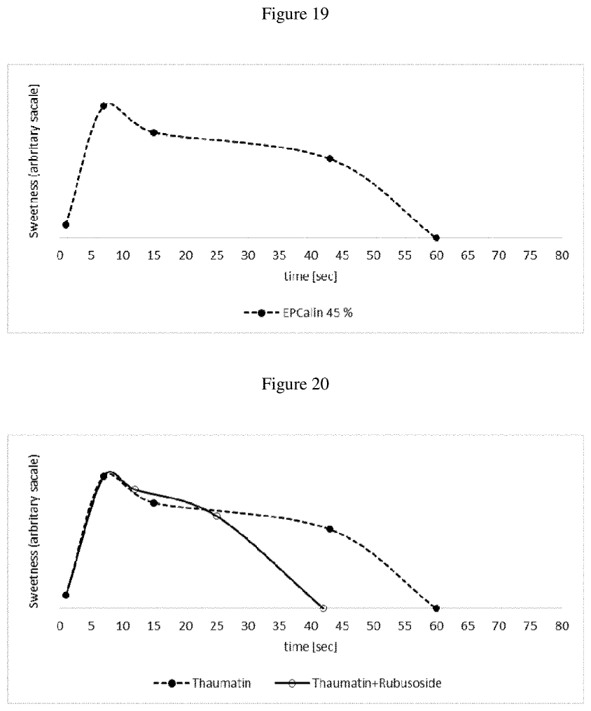

FIG. 19 denotes the sweetness/time profile for a 50 ppm solution of EPCalin 45%.

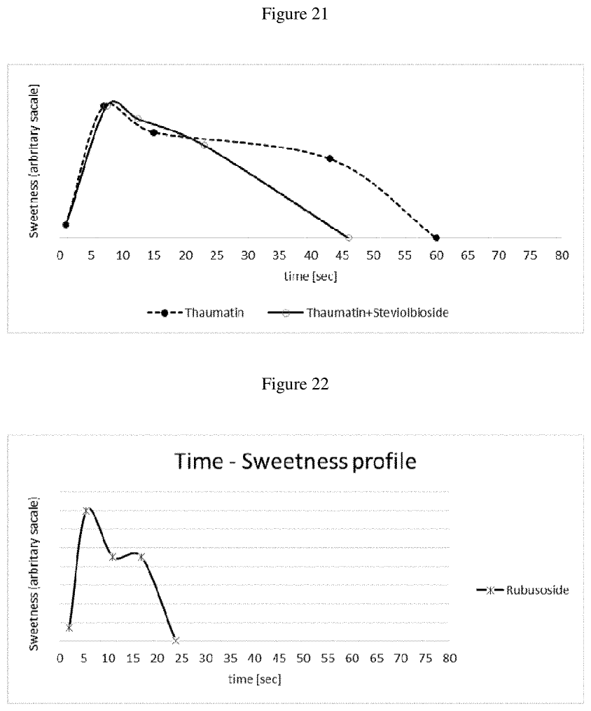

FIGS. 20 and 21 denote the sweetness/time profile for 50 ppm solution EPCalin 45% with either 90 ppm rubusoside (FIG. 20) or 90 ppm steviolbioside (FIG. 21).

As seen from the test results (FIGS. 19 through 21), rubusoside and steviolbioside were shown to reduce lingering considerably by 18 and 14 seconds with rubusoside and steviolbioside, respectively.

Flavor Modifying Properties of Steviolmonoside, Steviolbioside, Dulcoside and Rubusoside

For the tests following samples were prepared.

Reference sample: A commercial Energy Drink.

Test sample 1: The Energy Drink was diluted 8:2 with carbonated water (80 ml Drink+20 ml carbonated water).

Test samples 2 a-d: The Energy Drink was diluted 8:2 with carbonated water (80 ml Drink+20 ml carbonated water). Steviolmonoside (a), steviolbioside (b), dulcoside (c) or rubusoside (d) were added to the diluted Energy drink at concentrations of 65, 100, 160 or 100 μm.

24 test persons were chosen to establish with a 95% probability (100-β) a scenario where 50% of the panelists (pd) can recognize an existing difference at a significance level α=0.05.

The test persons were randomly allocated to following sequences of the two samples A and B: ABB, BAA, AAB, ABA and BAB.

The samples were marked with random 3 digit numbers.

Correct identification of the different samples by test persons were counted and compared to the total number of test persons. The statistical determination was based on published Tables for the minimum required correct answers.

This test design is a triangle test with a 3-AFC test design (3 Alternative Forced Choice Test).

TABLE
Test Design for triangle tests with a 3-AFC test design

| Target | Test | A | B |
|---|---|---|---|
| Recognition of Difference | #1 | Reference Sample | Test sample 1 |
| | #2 | Reference Sample | Test Sample 2a |
| | #3 | Reference Sample | Test Sample 2b |
| | #4 | Reference Sample | Test Sample 2c |
| | #5 | Reference Sample | Test Sample 2d |
| Recognition of Difference | #6 | Test sample 1 | Test Sample 2a |
| | #7 | Test sample 1 | Test Sample 2b |
| | #8 | Test sample 1 | Test Sample 2c |
| | #9 | Test sample 1 | Test Sample 2c |

TABLE
Results for tests investigating the degree of sweetness

| | Correctly identified | | |
|---|---|---|---|
| | and rated sweeter/ | | |
| Test | total number | Statistical | |
| # | of test persons | significance | Conclusion |
| #1 | 21/24 | significant | Reference Sample is |
| | | (p < 0.01) | sweeter |
| #2 | 14/24 | significant | Reference Sample is |
| | | (p < 0.05) | sweeter |
| #3 | 12/24 | significant | Reference Sample is |
| | | (p < 0.10) | sweeter (p = 0.05), Both are |
| | | | equally sweet (p = 0.10) |
| #4 | 17/24 | significant | Reference Sample is |
| | | (p < 0.01) | sweeter |
| #5 | 12/24 | significant | Reference Sample is |
| | | (p < 0.10) | sweeter (p = 0.05), Both are |
| | | | equally sweet (p = 0.10) |
| #6 | 19/24 | significant | Test Sample 2a is sweeter |
| | | (p < 0.01) | |
| #7 | 21/24 | significant | Test Sample 2b is sweeter |
| | | (p < 0.01) | |
| #8 | 18/24 | significant | Test Sample 2c is sweeter |
| | | (p < 0.01) | |
| #9 | 23/24 | significant | Test Sample 2d is sweeter |
| | | (p < 0.01) | |

These tests show that the flavor modifying properties of all 4 tested increase the perceived sweetness of an Energy Drink. It was also shown that steviolbioside (100 ppm) and (rubusoside 100 ppm) exert a more pronounced effect when compared to steviolmonoside (65 ppm) and dulcoside B (160 ppm).

As the above concentrations were chosen on basis of being less sweet than a 1.5% sugar solution and without mentionable off-tastes (i.e. bitterness), the test results are valid for a flavor use of the compounds tested.

Test for Equisweetness of Rubusoside and Steviolbioside with 1.5% Sucrose

As above, 24 test persons were chosen to establish with a 95% probability (100-β) a scenario where 50% of the panelists (pd) can recognize an existing difference at a significance level α=0.05.

The test persons were randomly allocated to following sequences of the two samples A and B: ABB, BAA, AAB, ABA and BAB

The table below provides results for the samples tested in 2 independent trials.

TABLE
Test Design for triangle tests

| Target | Test | A¹⁾ | B |
|---|---|---|---|
| Recognition | #1 | 110 ppm rubusoside 90% | 1.5% sucrose |
| of difference | | | |
| Recognition | #2 | 100 ppm steviolbioside 90% | 1.5% sucrose |
| of difference | | | |

¹⁾concentrations were chosen on basis of pre-tests with 5 test persons

Results

In Test #1 4/24 identified the different samples and rate it as sweeter. The corresponding test result for test 2 was 7/24.

The tests show conclusively that 110 ppm rubusoside 90% and 100 ppm steviolbioside 90% are less or equal in sweetness in comparison to 1.5% sucrose.

Threshold Tests for Steviolmonoside and Dulcoside B

In pre-tests, the test persons failed to identify a concentration with an equi-sweetness to a 1.5% sucrose solution. On one hand the sweetness of both substances is rather low and on the other hand bitterness (and other off-tastes) are very strong.

For steviolmonoside concentrations ≥200 ppm, the taste is very bitter and unpleasant while sweetness remains significantly below a 1.5% sucrose solution. For dulcoside B concentrations ≥400 ppm, the is taste bitter and unpleasant while sweetness remains significantly below a 1.5% sucrose solution.

By serial dilution, a bitterness threshold value for steviolmonoside and dulcoside B of 65 ppm and 160 ppm, respectively, could be established.

At this concentration both substances can be included in a flavor without imparting sweetness or bitter taste.

Time/Sweetness Profile of Rubusoside and Steviolbioside

Solutions of 250 ppm rubusoside 90% and steviolbioside 90% were prepared.

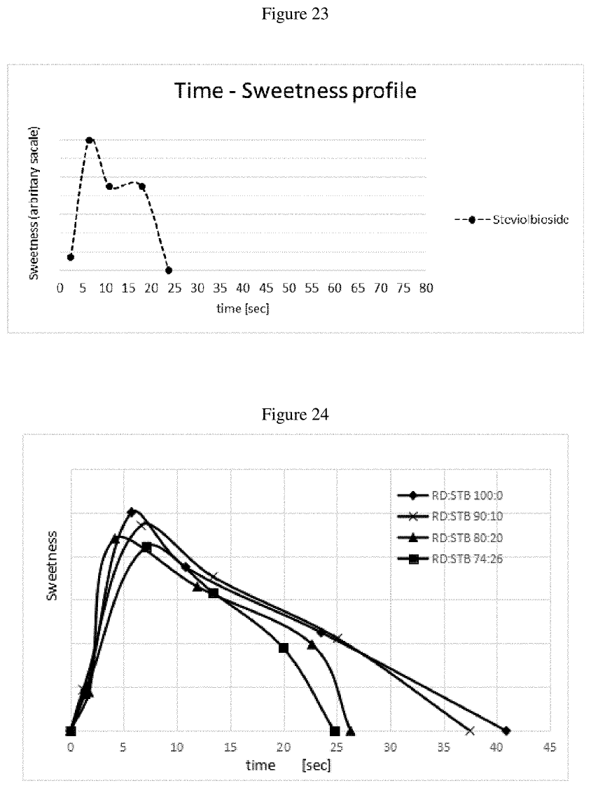

FIGS. 22 and 23 provide the sweetness time profile for rubusoside (90%) and steviolbioside (90%).

### Example 5 the Improvement of Steviolbioside (STB) to the Sweetness Profile of Rebaudioside D (RD)

Materials

STB is available from Sweet Green Fields and is produced according to the method described in Example 4.

RD (90%) is available from Sweet Green Fields.

Common Process:

STB and RD were weighed and uniformly mixed according to the weight shown in Table 5-1. Pure water was added to make the total volume to 100 ml, and subjected to a sensory evaluation test.

TABLE 5-1

| # | The ratio of RD to STB | Weight of RD (mg) | Weight of STB (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 7.40 | 10.80 | 23.50 | 34.00 |
| | person 2 | 2.53 | 4.09 | 10.84 | 23.57 | 47.80 |
| | mean value | 1.57 | 5.75 | 10.82 | 23.54 | 40.90 |
| 2 | person 1 | 0.90 | 7.00 | 11.70 | 20.80 | 28.00 |
| | person 2 | 1.50 | 6.29 | 15.10 | 29.34 | 47.00 |
| | mean value | 1.20 | 6.65 | 13.40 | 25.07 | 37.50 |
| 3 | person 1 | 1.10 | 4.70 | 11.00 | 17.30 | 24.10 |
| | person 2 | 2.40 | 3.65 | 12.82 | 28.04 | 28.50 |
| | mean value | 1.75 | 4.18 | 11.91 | 22.67 | 26.30 |
| 4 | person 1 | 1.30 | 7.40 | 11.90 | 15.80 | 22.90 |
| | person 2 | 1.38 | 6.90 | 14.90 | 24.10 | 26.63 |
| | mean value | 1.34 | 7.15 | 13.40 | 19.95 | 24.77 |

The sweetness profiles for different ratios of RD and STB based on the test data above was as shown in FIG. 24. The results showed that the lingering of RD was shortened as the content of STB was increased. The effect of decreased lingering was especially obvious when the ratio of STB to RD was higher than 20:80.

### Example 6 the Improvement of Dulcoside B (DB) to the Sweetness Profile of Rebaudioside D (RD)

Materials:

DB is available from Sweet Green Fields and is produced according to the method described in Example 4.

RD (90%) is available from Sweet Green Fields.

Common Process:

DB and RD were weighed and uniformly mixed according to the weight shown in Table 6-1. Pure water was added to make the total volume to 100 ml, and subjected to a sensory evaluation test.

TABLE 6-1

| # | The ratio of RD to DB | Weight of RD (mg) | Weight of DB (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 7.40 | 10.80 | 23.50 | 34.00 |
| | person 2 | 2.53 | 4.09 | 10.84 | 23.57 | 47.80 |
| | mean value | 1.57 | 5.75 | 10.82 | 23.54 | 40.90 |
| 2 | person 1 | 1.10 | 7.40 | 10.50 | 21.10 | 28.00 |
| | person 2 | 0.80 | 7.50 | 10.00 | 18.10 | 25.60 |
| | mean value | 0.95 | 7.45 | 10.25 | 19.60 | 26.80 |
| 3 | person 1 | 0.60 | 9.00 | 12.30 | 20.50 | 27.30 |
| | person 2 | 0.90 | 7.70 | 11.20 | 17.40 | 27.10 |
| | mean value | 0.75 | 8.35 | 11.75 | 18.95 | 27.20 |
| 4 | person 1 | 0.90 | 7.50 | 10.60 | 17.90 | 23.20 |
| | person 2 | 0.60 | 6.30 | 11.00 | 23.80 | 29.20 |
| | mean value | 0.75 | 6.90 | 10.80 | 20.85 | 26.20 |

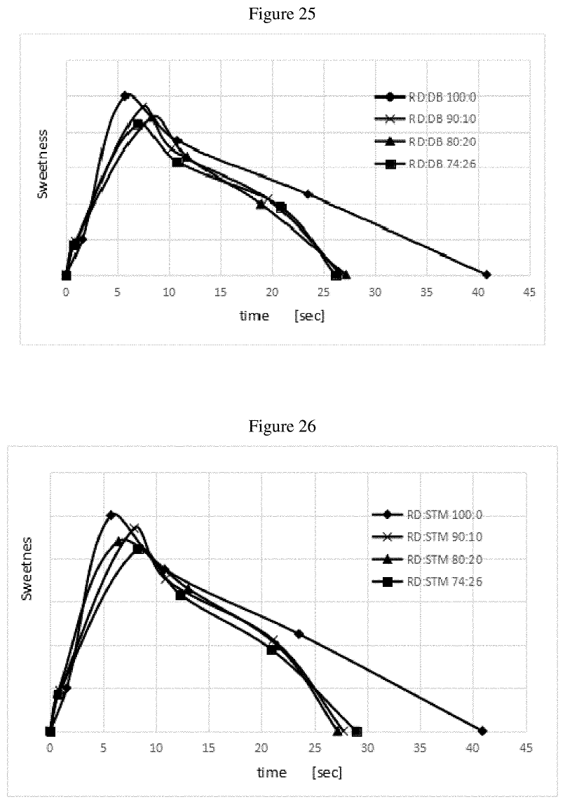

The sweetness profiles for different ratios of DB and RD based on the test data above was as shown in FIG. 25. The results showed that, the lingering of RD changed from 41 seconds to 27 seconds when DB was added. The effect of decreased lingering was significant and especially obvious when the ratio of DB to RD was higher than 10:90.

### Example 7 the Improvement of Steviolmonoside (STM) to the Sweetness Profile of Rebaudioside D (RD)

Materials:

STM is available from Sweet Green Fields and is produced according to the method described in Example 4.

RD (90%) is available from Sweet Green Fields.

Common Process:

STM and RD were weighed and uniformly mixed according to the weight shown in Table 7-1. Pure water was added to make the total volume to 100 ml, and subjected to a sensory evaluation test.

TABLE 7-1

| | The ratio | | |
|---|---|---|---|
| # | of RD to STM | Weight of RD (mg) | Weight of STM (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 7.40 | 10.80 | 23.50 | 34.00 |
| | person 2 | 2.53 | 4.09 | 10.84 | 23.57 | 47.80 |
| | mean value | 1.57 | 5.75 | 10.82 | 23.54 | 40.90 |
| 2 | person 1 | 1.20 | 8.40 | 11.50 | 21.50 | 30.70 |
| | person 2 | 0.60 | 7.50 | 10.30 | 20.70 | 24.80 |
| | mean value | 0.90 | 7.95 | 10.90 | 21.10 | 27.75 |
| 3 | person 1 | 0.80 | 4.60 | 12.70 | 25.70 | 30.80 |
| | person 2 | 0.60 | 8.30 | 13.40 | 17.30 | 23.60 |
| | mean value | 0.70 | 6.45 | 13.05 | 21.50 | 27.20 |
| 4 | person 1 | 0.70 | 8.70 | 12.85 | 22.40 | 34.10 |
| | person 2 | 0.80 | 7.90 | 11.80 | 19.40 | 23.80 |
| | mean value | 0.75 | 8.30 | 12.33 | 20.90 | 28.95 |

The sweetness profiles for different ratios of STM and RD based on the test data above was as shown in FIG. 26. The results showed that, the lingering of RD changed from 41 seconds to 28 seconds when STM was added. Therefore, STM had a good effect of decreased lingering on RD. The effect of decreased lingering was significant and especially obvious when the ratio of STM to RD was higher than 10:90.

### Example 8 the Improvement of Rubusoside (RU) to the Sweetness Profile of Rebaudioside (RD)

Materials:

Rubososide (90%) is available from LAYN, China.

RD (90%) is available from Sweet Green Fields.

Common Process:

RU and RD were weighed and uniformly mixed according to the weight shown in Table 8-1. Pure water was added to make the total volume to 100 ml, and subjected to a sensory evaluation test.

TABLE 8-1

| # | The ratio of RD to RU | Weight of RD (mg) | Weight of RU (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 7.40 | 10.80 | 23.50 | 34.00 |
| | person 2 | 2.53 | 4.09 | 10.84 | 23.57 | 47.80 |
| | mean value | 1.57 | 5.75 | 10.82 | 23.54 | 40.90 |
| 2 | person 1 | 0.90 | 7.70 | 11.70 | 19.70 | 28.60 |
| | person 2 | 2.60 | 4.06 | 7.98 | 15.35 | 23.52 |
| | mean value | 1.75 | 5.88 | 9.84 | 17.53 | 26.06 |
| 3 | person 1 | 1.10 | 7.40 | 10.50 | 19.40 | 27.90 |
| | person 2 | 2.50 | 5.20 | 10.02 | 17.59 | 26.49 |
| | mean value | 1.80 | 6.30 | 10.26 | 18.50 | 27.20 |
| 4 | person 1 | 1.20 | 8.40 | 11.30 | 21.00 | 27.90 |
| | person 2 | 2.83 | 6.33 | 12.33 | 24.68 | 27.90 |
| | mean value | 2.02 | 7.37 | 11.82 | 22.84 | 27.90 |

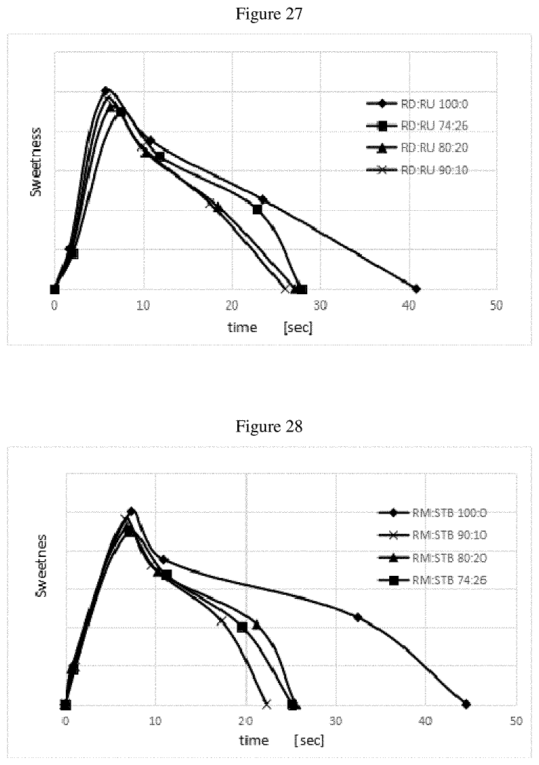

The sweetness profiles for different ratios of RU and RD based on the test data above was as shown in FIG. 27. The results showed that the lingering of RD changed from 41 seconds to 28 seconds when RU added. Therefore, RU had a good effect of decreased lingering on RD. The effect of decreased lingering was significant and especially obvious when the ratio of RU to RD was higher than 10:90.

### Example 9 the Improvement of Steviolbioside (STB) to the Sweetness Profile of Rebaudioside M (RM)

Materials:

STB is available from Sweet Green Fields and is produced according to the method described in Example 4.

RM(90%) is available from Sichuan Ingia Biosynthetic Co., ltd, China.

Common Process:

STB and RM were weighed and uniformly mixed according to the weight shown in Table 9-1. Pure water was added to make the total volume to 100 ml, and subjected to a sensory evaluation test.

TABLE 9-1

| # | The ratio of RM to STB | Weight of RM (mg) | Weight of STB (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.90 | 7.60 | 11.00 | 33.00 | 39.00 |
| | person 2 | 0.90 | 7.10 | 10.90 | 31.90 | 50.00 |
| | mean value | 0.90 | 7.35 | 10.95 | 32.45 | 44.50 |
| 2 | person 1 | 1.10 | 6.90 | 11.00 | 18.50 | 25.00 |
| | person 2 | 1.20 | 6.30 | 8.02 | 16.20 | 19.80 |
| | mean value | 1.15 | 6.60 | 9.51 | 17.35 | 22.40 |
| 3 | person 1 | 0.60 | 7.10 | 11.70 | 25.40 | 30.20 |
| | person 2 | 0.70 | 6.50 | 8.90 | 17.10 | 21.00 |
| | mean value | 0.65 | 6.80 | 10.30 | 21.25 | 25.60 |
| 4 | person 1 | 0.90 | 7.20 | 12.50 | 22.20 | 30.10 |
| | person 2 | 0.80 | 6.90 | 9.90 | 17.00 | 20.30 |
| | mean value | 0.85 | 7.05 | 11.20 | 19.60 | 25.20 |

The sweetness profiles for different ratios of STB and RM based on the test data above was as shown in FIG. 28. The results showed that the lingering of RM was shortened when STM was added. Therefore, STM showed a good effect of decreased lingering on RM. The effect of decreased lingering was significant and especially obvious when the ratio of STB to RM was higher than 10:90.

### Example 10 the Improvement of Dulcoside B (DB) to the Sweetness Profile of Rebaudioside (RM)

Materials:

DB is available from Sweet Green Fields and is produced according to the method described in Example 4.

RM(90%) is available from Sichuan Ingia Biosynthetic Co., ltd, China.

Common Process:

DB and RM were weighed and uniformly mixed according to the weight shown in Table 10-1. Pure water was added to make the total volume to 100 ml, and subjected to a sensory evaluation test.

TABLE 10-1

| # | The ratio of RM to DB | Weight of RM (mg) | Weight of DB (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.90 | 7.60 | 11.00 | 33.00 | 39.00 |
| | person 2 | 0.90 | 7.10 | 10.90 | 31.90 | 50.00 |
| | mean value | 0.90 | 7.35 | 10.95 | 32.45 | 44.50 |
| 2 | person 1 | 0.60 | 7.10 | 10.90 | 21.60 | 29.40 |
| | person 2 | 0.20 | 7.30 | 9.90 | 13.60 | 21.50 |
| | mean value | 0.40 | 7.20 | 10.40 | 17.60 | 25.45 |
| 3 | person 1 | 0.50 | 7.10 | 12.70 | 21.90 | 32.00 |
| | person 2 | 0.40 | 7.50 | 13.00 | 16.50 | 22.80 |
| | mean value | 0.45 | 7.30 | 12.85 | 19.20 | 27.40 |
| 4 | person 1 | 0.40 | 12.80 | 17.40 | 33.00 | 41.90 |
| | person 2 | 0.60 | 7.10 | 13.30 | 17.60 | 20.00 |
| | mean value | 0.50 | 9.95 | 15.35 | 25.30 | 30.95 |

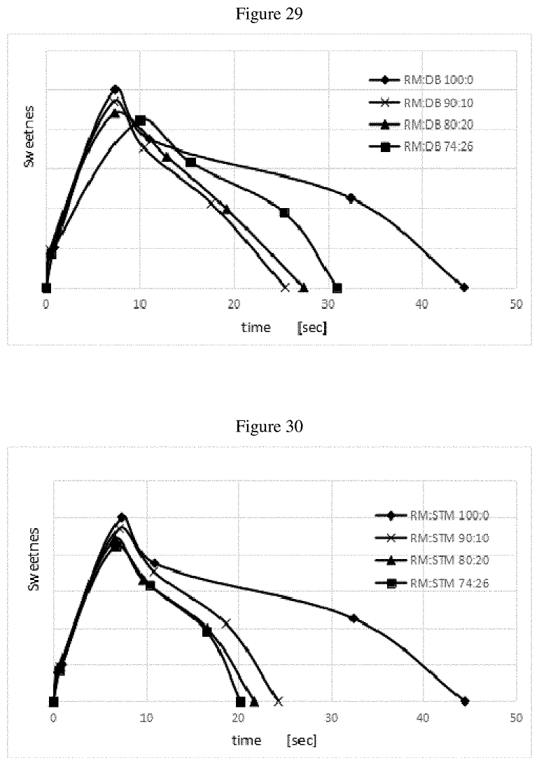

The sweetness profiles for different ratios of DB and RM based on the test data above was as shown in FIG. 29. The results showed that the lingering of RM was shortened when DB was added. Therefore, DB had a good effect of decreased lingering on RM. The effect of decreased lingering was significant and especially obvious when the ratio of DB to RM was higher than 10:90.

### Example 11 the Improvement of Steviolmonoside (STM) to the Sweetness Profile of Rebaudioside M (RM)

Materials:

STM is available from Sweet Green Fields and is produced according to the method described in Example 4.

RM(90%) is available from Sichuan Ingia Biosynthetic Co., ltd, China.

Common Process:

STM and RM were weighed and uniformly mixed according to the weight shown in Table 11-1. Pure water was added to make the total volume to 100 ml, and subjected to a sensory evaluation test.

TABLE 11-1

| | | Weight | |
|---|---|---|---|
| # | The ratio of RM to STM | of RM (mg) | Weight of STM (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.90 | 7.60 | 11.00 | 33.00 | 39.00 |
| | person 2 | 0.90 | 7.10 | 10.90 | 31.90 | 50.00 |
| | mean value | 0.90 | 7.35 | 10.95 | 32.45 | 44.50 |
| 2 | person 1 | 0.70 | 7.10 | 10.90 | 18.70 | 23.70 |
| | person 2 | 0.80 | 7.10 | 10.80 | 18.60 | 24.90 |
| | mean value | 0.75 | 7.10 | 10.85 | 18.65 | 24.30 |
| 3 | person 1 | 0.40 | 7.10 | 10.60 | 16.20 | 21.00 |
| | person 2 | 0.60 | 6.20 | 8.90 | 17.10 | 22.40 |
| | mean value | 0.50 | 6.65 | 9.75 | 16.65 | 21.70 |
| 4 | person 1 | 0.70 | 6.80 | 9.70 | 13.60 | 17.90 |
| | person 2 | 0.50 | 6.60 | 11.10 | 19.60 | 22.60 |
| | mean value | 0.60 | 6.70 | 10.40 | 16.60 | 20.25 |

The sweetness profiles for different ratios of STM and RM based on the test data above was as shown in FIG. 30. The results showed that the lingering of RM was shortened when STM was added. Therefore, STM had a good effect of decreased lingering on RM. The effect of decreased lingering was significant and especially obvious when the ratio of STM to RM was higher than 10:90.

### Example 12 the Improvement of Rubusoside (RU) to the Sweetness Profile of Rebaudioside M (RM)

Materials:

Rubososide (90%) is available from LAYN, China.

RM(90%) is available from Sichuan Ingia Biosynthetic Co., ltd, China.

Common Process:

RU and RM were weighed and uniformly mixed according to the weight shown in Table 12-1. Pure water was added to make the total volume to 100 ml, and subjected to a sensory evaluation test.

TABLE 12-1

| # | The ratio of RM to RU | Weight of RM (mg) | Weight of RU (mg) |
|---|---|---|---|
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.90 | 7.60 | 11.00 | 33.00 | 39.00 |
| | person 2 | 0.90 | 7.10 | 10.90 | 31.90 | 50.00 |
| | mean value | 0.90 | 7.35 | 10.95 | 32.45 | 44.50 |
| 2 | person 1 | 0.50 | 6.90 | 10.50 | 16.80 | 24.50 |
| | person 2 | 2.60 | 4.06 | 7.98 | 15.35 | 23.52 |
| | mean value | 1.55 | 5.48 | 9.24 | 16.08 | 24.01 |
| 3 | person 1 | 0.40 | 7.20 | 10.60 | 17.40 | 22.50 |
| | person 2 | 0.60 | 6.90 | 8.10 | 18.90 | 21.60 |
| | mean value | 0.50 | 7.05 | 9.35 | 18.15 | 22.05 |
| 4 | person 1 | 0.60 | 7.90 | 11.40 | 20.80 | 29.70 |
| | person 2 | 0.20 | 6.90 | 7.40 | 14.40 | 17.50 |
| | mean value | 0.40 | 7.40 | 9.40 | 17.60 | 23.60 |

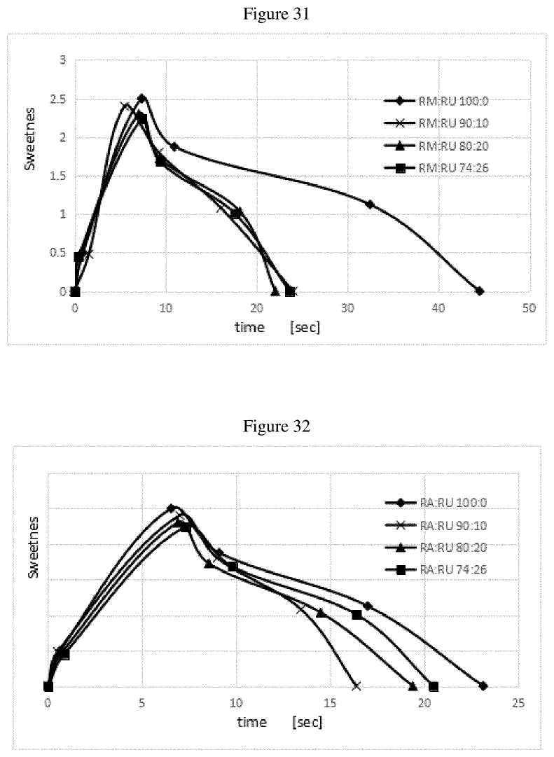

The sweetness profiles for different ratios of RU and RM based on the test data above was as shown in FIG. 31. The results showed that the lingering of RM changed from 44 seconds to 28 seconds when RU was added. Therefore, RU had a good effect of decreased lingering on RM. The effect of decreased lingering was significant and especially obvious when the ratio of STM to RM was higher than 10:90.

### Example 13 the Improvement of Rubusoside (RU) to the Sweetness Profile of Rebaudioside a 97% (RA97)

Materials:

Rubososide (90%) is available from LAYN, China.

RA97 (97%) is available from Sweet Green Fields.

Common Process:

RA97 and RU were weighed and uniformly mixed according to the weight shown in Table 13-1. Pure water was added to make the total volume to 100 ml, and subjected to a sensory evaluation test.

TABLE 13-1

| | | Weight | |
|---|---|---|---|
| | The ratio of RA97 to RU | of RA97 (mg) | Weight of RU (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test results A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| | | [sec] | [sec] | [sec] | [sec] | [sec] |
| 1 | person 1 | 0.60 | 6.80 | 9.40 | 18.30 | 23.69 |
| 1 | person 2 | 0.70 | 6.30 | 8.80 | 15.70 | 22.60 |
| | mean value | 0.65 | 6.55 | 9.10 | 17.00 | 23.15 |
| 2 | person 1 | 0.40 | 7.20 | 9.00 | 13.50 | 17.70 |
| 2 | person 2 | 0.60 | 6.80 | 9.00 | 13.40 | 15.10 |
| | mean value | 0.50 | 7.00 | 9.00 | 13.45 | 16.40 |
| 3 | person 1 | 0.60 | 7.10 | 9.10 | 14.60 | 19.30 |
| 3 | person 2 | 0.80 | 6.70 | 8.00 | 14.40 | 19.50 |
| | mean value | 0.70 | 6.90 | 8.55 | 14.50 | 19.40 |
| 4 | person 1 | 1.10 | 7.70 | 10.50 | 15.60 | 21.00 |
| 4 | person 2 | 0.60 | 6.90 | 9.10 | 17.20 | 20.00 |
| | mean value | 0.85 | 7.30 | 9.80 | 16.40 | 20.50 |

The sweetness profiles for different ratios of RA97 and RU based on the test data above was as shown in FIG. 32. The results showed that the lingering of RA97 was shortened when RU added. The effect of decreased lingering was significant when the ratio of RU to RA was higher than 10:90. Especially when the ratio of RU to RM was 10:90, the sweet lingering of RA was shortened very obviously.

### Example 14 the Improvement of Steviolbioside (STB) to the Sweetness Profile of RA97

STB is available from Sweet Green Fields and is produced according to the method described in Example 4.

RA97 is available from Sweet Green Fields.

Common Process:

RA97 and STB were weighed and uniformly mixed according to the weight shown in Table 14-1. Pure water was added to make the total volume to 100 ml, and subjected to a sensory evaluation test

TABLE 14-1

| | | Weight of RA97 | |
|---|---|---|---|
| | The ratio of RA97 to STB | (mg) | Weight of STB (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| | | [sec] | [sec] | [sec] | [sec] | [sec] |
| 1 | person 1 | 0.60 | 6.80 | 9.40 | 18.30 | 23.69 |
| 1 | person 2 | 0.70 | 6.30 | 8.80 | 15.70 | 22.60 |
| | mean value | 0.65 | 6.55 | 9.10 | 17.00 | 23.15 |
| 2 | person 1 | 0.90 | 6.50 | 9.10 | 14.50 | 18.50 |
| 2 | person 2 | 1.10 | 6.20 | 8.10 | 13.00 | 17.20 |
| | mean value | 1.00 | 6.35 | 8.60 | 13.75 | 17.85 |
| 3 | person 1 | 0.40 | 6.90 | 9.50 | 13.90 | 18.00 |
| 3 | person 2 | 0.70 | 6.50 | 8.90 | 14.20 | 16.90 |
| | mean value | 0.55 | 6.70 | 9.20 | 14.05 | 17.45 |
| 4 | person 1 | 1.10 | 6.60 | 9.40 | 14.80 | 17.10 |
| 4 | person 2 | 0.90 | 6.20 | 9.10 | 14.60 | 17.20 |
| | mean value | 1.00 | 6.40 | 9.25 | 14.70 | 17.15 |

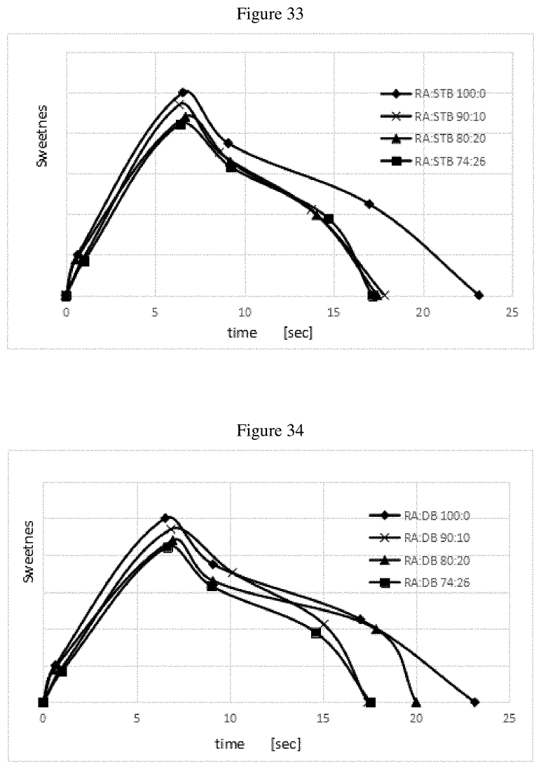

The sweetness profiles for different ratios of RA97 and STB based on the test data above was shown in FIG. 33. The results showed that the lingering of RA97 was shortened when STB was added. The effect of decreased lingering was significant when the ratio of STB to RA was higher than 10:90.

### Example 15 the Improvement of Dulcoside B (DB) to the Sweetness Profile of RA97

DB is available from Sweet Green Fields and is produced according to the method described in Example 4.

RA97 is available from Sweet Green Fields.

Common Process:

RA97 and DB were weighed and uniformly mixed according to the weight shown in Table 15-1. Pure water was added to make the total volume to 100 ml, and subjected to a sensory evaluation test.

TABLE 15-1

| | | Weight of RA97 | |
|---|---|---|---|
| # | The ratio of DB to STB | (mg) | Weight of DB (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 6.80 | 9.40 | 18.30 | 23.69 |
| | person 2 | 0.70 | 6.30 | 8.80 | 15.70 | 22.60 |
| | mean value | 0.65 | 6.55 | 9.10 | 17.00 | 23.15 |
| 2 | person 1 | 0.90 | 6.80 | 11.40 | 17.00 | 19.00 |
| | person 2 | 1.10 | 6.90 | 8.90 | 13.10 | 15.80 |
| | mean value | 1.00 | 6.85 | 10.15 | 15.05 | 17.40 |
| 3 | person 1 | 0.40 | 6.70 | 9.70 | 18.10 | 20.80 |
| | person 2 | 0.70 | 7.20 | 8.50 | 17.60 | 19.20 |
| | mean value | 0.55 | 6.95 | 9.10 | 17.85 | 20.00 |
| 4 | person 1 | 1.10 | 6.50 | 8.90 | 15.10 | 18.10 |
| | person 2 | 0.90 | 6.80 | 9.10 | 14.10 | 17.00 |
| | mean value | 1.00 | 6.65 | 9.00 | 14.60 | 17.55 |

The sweetness profile for different ratios of RA97 and DB based on the test data above was shown in FIG. 34. The results showed that the lingering of RA97 was shortened when DB was added. The effect of decreased lingering was significant when the ratio of DB to RA was higher than 10:90.

### Example 16 the Improvement of Steviolmonoside (STM) to the Sweetness Profile of RA97

STM is available from Sweet Green Fields and is produced according to the method described in Example 4.

RA97 is available from Sweet Green Fields.

Common Process:

RA97 and STM were weighed and uniformly mixed according to the weight shown in Table 16-1. Pure water was added to make the total volume to 100 ml, and subjected to a sensory evaluation test.

TABLE 16-1

| | | Weight of RA97 | Weight of STM |
|---|---|---|---|
| # | The ratio of STM to STM | (mg) | (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 6.80 | 9.40 | 18.30 | 23.69 |
| | person 2 | 0.70 | 6.30 | 8.80 | 15.70 | 22.60 |
| | mean value | 0.65 | 6.55 | 9.10 | 17.00 | 23.15 |
| 2 | person 1 | 0.90 | 7.20 | 9.00 | 13.50 | 17.70 |
| | person 2 | 1.10 | 6.70 | 8.00 | 14.40 | 19.50 |
| | mean value | 1.00 | 6.95 | 8.50 | 13.95 | 18.60 |
| 3 | person 1 | 0.40 | 7.10 | 9.10 | 14.60 | 19.30 |
| | person 2 | 0.70 | 6.80 | 9.00 | 13.40 | 15.10 |
| | mean value | 0.55 | 6.95 | 9.05 | 14.00 | 17.20 |
| 4 | person 1 | 1.10 | 7.70 | 10.50 | 15.60 | 21.00 |
| | person 2 | 0.90 | 8.00 | 9.20 | 14.00 | 19.60 |
| | mean value | 1.00 | 7.85 | 9.85 | 14.80 | 20.30 |

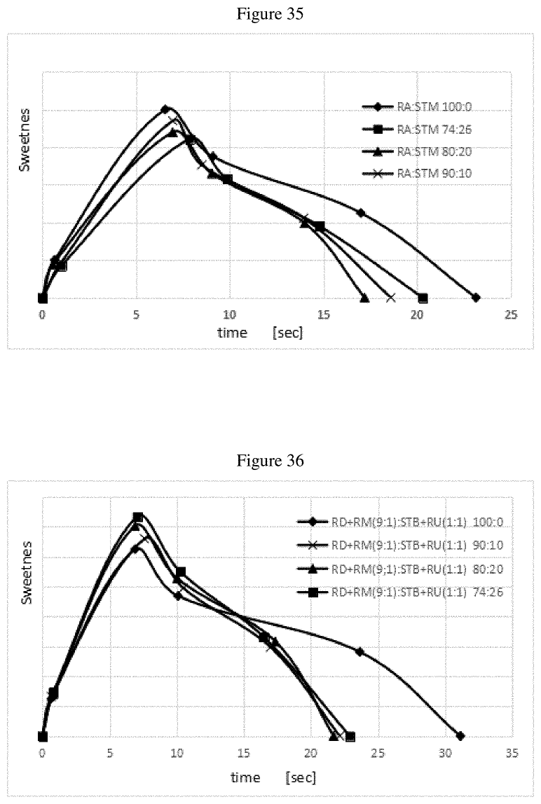

The sweetness profiles for different ratios of RA97 and STM based on the test data above was shown in FIG. 35. The results showed that the lingering of RA97 was shortened when STM was added. The effect of decreased lingering was significant when the ratio of STM to RA was higher than 10:90.

### Example 17 the Improvement of STB+RU(1/1) to the Sweetness Profile of RD+RM (9/1)

STB is available from Sweet Green Fields and is produced according to the method described in Example 4.

RD (90%) is available from Sweet Green Fields.

Rubososide (90%) is available from LAYN, China.

RM(90%) is available from Sichuan Ingia Biosynthetic Co., ltd, China.

Common Process:

STB+RU(1/1) and RD+RM (9/1) were weighed and uniformly mixed according to the weight shown in Table 17-1. Pure water was added to make the total volume to 100 ml, and subjected to a sensory evaluation test.

TABLE 17-1

| | | Weight of RD + RM | Weight of STB + |
|---|---|---|---|
| | The ratio of RD + RM | (9/1) | RU (1/1) |
| # | (9/1) to STB + RU(1/1) | solution (mg) | solution (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 6.80 | 9.90 | 27.20 | 32.40 |
| | person 2 | 0.70 | 7.00 | 10.30 | 20.10 | 29.90 |
| | mean value | 0.65 | 6.90 | 10.10 | 23.65 | 31.15 |
| 2 | person 1 | 0.90 | 7.70 | 10.50 | 16.70 | 20.30 |
| | person 2 | 0.40 | 7.50 | 10.50 | 17.30 | 24.00 |
| | mean value | 0.65 | 7.60 | 10.50 | 17.00 | 22.15 |
| 3 | person 1 | 0.90 | 7.30 | 10.30 | 18.40 | 22.00 |
| | person 2 | 0.70 | 6.50 | 9.80 | 16.30 | 21.40 |
| | mean value | 0.80 | 6.90 | 10.05 | 17.35 | 21.70 |
| 4 | person 1 | 0.70 | 7.00 | 10.10 | 17.00 | 24.40 |
| | person 2 | 0.90 | 7.10 | 10.40 | 16.00 | 21.40 |
| | mean value | 0.80 | 7.05 | 10.25 | 16.50 | 22.90 |

The sweetness profiles for different ratios of STB+RU(1/1) and RD+RM (9/1) based on the test data above was as shown in FIG. 36. The results showed that the lingering of the composition of RM/RD changed from 31 seconds to 22 seconds when STB+RU(1/1) was added. Therefore, STB+RU(1/1) had a good effect of decreased lingering on RD+RM (9/1). The effect of decreased lingering was significant when the ratio of STB+RU(1/1) to RD+RM (9/1) was higher than 10:90.

### Example 18 the Improvement of STB+STM(2/3) to the Sweetness Profile of RD+RM (5/5)

STB and STM are available from Sweet Green Fields and are produced according to the method described in Example 4.

RD (90%) is available from Sweet Green Fields.

RM(90%) is available from Sichuan Ingia Biosynthetic Co., ltd, China.

Common Process:

STB+STM(2/3) and RD+RM (5/5) were weighed and uniformly mixed according to the weight shown in Table 18-1. Pure water was added to make the total volume to 100 ml, and subjected to a sensory evaluation test.

TABLE 18-1

| | | Weight of RD + | Weight of STB + |
|---|---|---|---|
| | The ratio of RD + RM (5/5) | RM | STM (2/3) |
| # | to STB + STM (2/3) | (5/5) (mg) | (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 0.60 | 8.60 | 10.80 | 15.80 | 24.30 |
| | person 2 | 0.70 | 7.20 | 10.90 | 20.00 | 26.10 |
| | mean value | 0.65 | 7.90 | 10.85 | 17.90 | 25.20 |
| 2 | person 1 | 0.90 | 7.80 | 8.90 | 15.80 | 21.10 |
| | person 2 | 1.10 | 7.10 | 8.10 | 16.00 | 21.60 |
| | mean value | 1.00 | 7.45 | 8.50 | 15.90 | 21.35 |
| 3 | person 1 | 0.40 | 6.70 | 9.00 | 17.10 | 24.00 |
| | person 2 | 0.70 | 7.10 | 9.10 | 15.20 | 21.20 |
| | mean value | 0.55 | 6.90 | 9.05 | 16.15 | 22.60 |
| 4 | person 1 | 0.60 | 6.80 | 10.10 | 19.70 | 26.60 |
| | person 2 | 0.70 | 6.70 | 10.00 | 16.40 | 21.00 |
| | mean value | 0.65 | 6.75 | 10.05 | 18.05 | 23.80 |

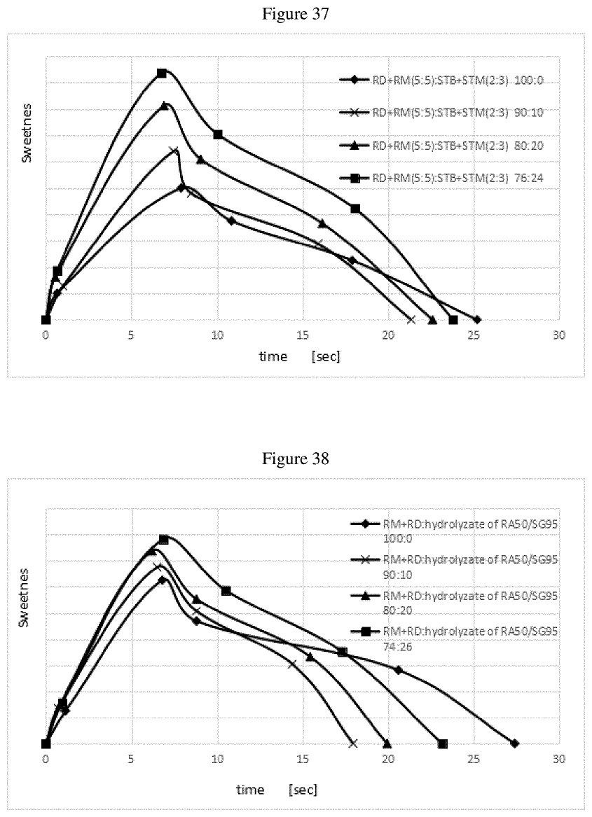

The sweetness profiles for different ratios of STB+STM(2/3) and RD+RM (5/5) based on the test data above was as shown in FIG. 37. The results showed that the lingering of the composition of RM/RD was shortened when STB+STM (2/3) added. The effect of decreased lingering was obvious when the ratio of STB+STM (2/3) to RD+RM (5/5) was higher than 10:90.

### Example 19 Method to Produce Hydrolyzate of RA50/SG95

16 g RA50/SG95 available from Sweet Green Fields (RA 53.95%, STV 35.2%, RC 3.39%, TSG(9SG) 95.9%) was dissolved into 140 ml pure water. 1 ml 20% NaOH was added into the reaction solution and heated to 95−100° C. for about 2 hours. Then the solution was cooled to room temperature and spray dried to provide a hydrolyzate of RA50/SG95 (RA 40.25%, STV 26.32%, RB 10.58%, STB 5.88%, TSG(9SG) 87.49%).

### Example 20 the Improvement of the Hydrolyzate of RA50/SG95 to the Sweet Profile of RM+RD(5/5)

Common Process:

The hydrolyzate of RA50/SG95 (the product of Example 19) and RM+RD(5/5) were weighed and uniformly mixed according to the weight shown in Table 20-1. Pure water was added to make the total volume to 100 ml, and subjected to a sensory evaluation test.

TABLE 20-1

| | The ratio of | | |
|---|---|---|---|
| | RM + RD (1/9) to | | |
| | the product of | Weight of | Weight of the product of |
| # | Example 19 | RM + RD (1/9) (mg) | Example 19 (mg) |
| 1 | 100/0 | 50 | 0 |
| 2 | 90/10 | 50 | 5.6 |
| 3 | 80/20 | 50 | 12.5 |
| 4 | 74/26 | 50 | 17.5 |

Sensory Evaluation Procedure:

The sample solutions were tested by a panel of two persons. Each person of the test panel drank different steviol glycoside solutions with defined concentrations. During the test, all persons had a time clock. They had to note the appearance-time for five specific points of a sweetness profile (onset, maximum sweetness, lingering on, lingering off and no taste).

Test Results

A mean value of all test person results was determined.

| | | | | LIN- | LIN- | |
|---|---|---|---|---|---|---|
| | | | | GERING | GERING | NO |
| | | ONSET | MAX | ON | OFF | TASTE |
| # | | [sec] | [sec] | [sec] | [sec] | [sec] |
| 1 | person 1 | 1.10 | 6.60 | 9.50 | 19.20 | 28.10 |
| | person 2 | 1.20 | 7.00 | 8.10 | 22.00 | 26.70 |
| | mean value | 1.15 | 6.80 | 8.80 | 20.60 | 27.40 |
| 2 | person 1 | 0.60 | 5.90 | 8.50 | 14.00 | 17.80 |
| | person 2 | 0.80 | 7.10 | 9.10 | 14.80 | 18.10 |
| | mean value | 0.70 | 6.50 | 8.80 | 14.40 | 17.95 |
| 3 | person 1 | 0.70 | 5.50 | 8.60 | 14.70 | 18.70 |
| | person 2 | 0.90 | 6.90 | 9.00 | 16.20 | 21.20 |
| | mean value | 0.80 | 6.20 | 8.80 | 15.45 | 19.95 |
| 4 | person 1 | 1.10 | 7.00 | 10.20 | 17.00 | 22.60 |
| | person 2 | 0.80 | 6.70 | 10.80 | 17.60 | 23.70 |
| | mean value | 0.95 | 6.85 | 10.50 | 17.30 | 23.15 |

The sweetness profiles for different ratios of the hydrolyzate of RA50/SG95 to RM+RD(5/5) based on the test data above was as shown in FIG. 38. The hydrolyzate of RA50/SG95 can be considered as a composition of steviol glycosides which contains high content of steviol glycosides with lower molecular weight. The results showed that the lingering of RM/RD composition was shortened when the hydrolyzate of RA50/SG95 was added. The effect of decreased lingering was obvious when the ratio of the hydrolyzate of RA50/SG95 to RD+RM (5/5) was higher than 10:90.

### Example 21 Method to Produce Stevia Composition Containing Rubusoside (RU) from a Stevia Composition Containing Stevioside by Using 5-Galactosidase

Materials:

Stevia composition containing stevioside: RA30/SG95 available from Sweet Green Fields which contains 33.12% RA and 58.07% STV as well as the total steviol glycosides is 98.33% TSG(9SG).

β-galactosidase: Lactase DS100 available from AMANO ENZYME INC.

Process:

50 g RA30/SG95 was dissolved into 100 ml pure water. The solution was stirred and heated to 40-45° C. 10 g β-galactosidase was added to the reaction solution. The solution was then stirred at 40-45° C. for about 36 hours. After the reaction was completed, the reaction solution was heated to 95-100° C. for 30 min to inactivate the enzyme. Then the solution was cooled to room temperature. The supernatant was separated by centrifugation at 4000 r/m for 10 min. The supernatant was the spray dried to provide the product. The composition of the product was 23.21% RA, 4.91% STV, 36.51% RU and the total steviol glycosides is 68.79% TSG(9SG).

### Example 22

In this example the taste improvement of the product of Example 21 to normal stevia extracts was evaluated.

Materials:

| Sample | Source | Specification |
|---|---|---|
| Enzyme- | The product of Example | 23.21% RA, 4.91% STV, |
| transformed | 21 | 36.51% RU and 68.79% |
| rubusoside | | TSG (9SG) |
| RA | Available from Sweet | 99.94% RA |
| | Green Fields | |
| RD | available from Sichuan | RD 94.39% |
| | Ingia Biosynthetic Co,.ltd, | |
| | China. | |
| RM | available from Sichuan | RM 93.03%, RD3.67% |
| | Ingia Biosynthetic Co,.ltd, | |
| | China. | |

Sample Solutions Preparation:

The enzyme-transformed RU (abbreviated as ET-RU below) and other stevia extracts were weighed and uniformly mixed according to the weight shown in the Tables below. Pure water was added to make the total volume to 100 ml, and subjected to a sensory evaluation test.

Sensory Evaluation Procedure:

For evaluation of the taste profile, the samples were tested by a panel of four people. The panel was asked to describe the taste profile and score values between 0-5 according to the increasing intensity of bitterness and metallic aftertaste. 1 trained taster tasted independently the samples first. The tester was allowed to re-taste, and then make notes for the sensory attributes perceived. Afterwards, another 3 tasters tasted the sample and the attributes were noted and discussed among the tasters to find a suitable description. In case that more than 1 taster disagreed with the result, the tasting was repeated. For example, a “5” for intensity of bitterness is the worst score for having a strong bitter taste and conversely a value of 0 or near zero means the bitterness is very slight. Similarly, a “5” for metallic aftertaste is not desired. A value of zero or near zero means that the metallic aftertaste is reduced or is removed.

For the sweet lingering, the sample solutions were tested by a panel of two persons. Each person of the test panel drank different steviol glycoside solutions with defined concentrations. During the test, all persons had a time clock. They had to note the time when sweetness disappears. The shorter time means the desired sweet lingering.

Results:

1. The improvement of enzyme-transformed RU to RA

TABLE 22-1
the weight of RA and ET-RU

| | The ratio | | |
|---|---|---|---|
| # | of RA to ET-RU | Weight of RA (mg) | Weight of ET-RU (mg) |
| 1-1 | 100/0 | 50 | 0 |
| 1-2 | 95/5 | 50 | 2.6 |
| 1-3 | 90/10 | 50 | 5.6 |
| 1-4 | 85/15 | 50 | 8.8 |
| 1-5 | 80/20 | 50 | 12.5 |
| 1-6 | 75/25 | 50 | 16.6 |

TABLE 22-2
the result in sensory evaluation

| # | Metallic aftertaste | bitterness | Sweet lingering |
|---|---|---|---|
| 1-1 | 4 | 2 | 37 s |
| 1-2 | 4 | 2 | 32 s |
| 1-3 | 4 | 2 | 34 s |
| 1-4 | 3 | 2 | 29 s |
| 1-5 | 3 | 1 | 30 s |
| 1-6 | 2 | 1 | 26 s |

The taste profile of RA can be improved by enzyme-transformed RU. As the ratio of ET-RU to RA increased, the bitterness and metallic aftertaste are reduced. The sweet lingering of RA can be shortened from about 37 seconds to about 30 seconds. Especially when the ratio of ET-RU to RA reached 25/75, the effect of decreased lingering was significant.

2. The improvement of enzyme-transformed RU to RD

TABLE 22-3
the weight of RD and ET-RU

| | | | Weight of ET-RU |
|---|---|---|---|
| # | The ratio of RD to ET-RU | Weight of RD (mg) | (mg) |
| 2-1 | 100/0 | 50 | 0 |
| 2-2 | 95/5 | 50 | 2.6 |
| 2-3 | 90/10 | 50 | 5.6 |
| 2-4 | 85/15 | 50 | 8.8 |
| 2-5 | 80/20 | 50 | 12.5 |
| 2-6 | 75/25 | 50 | 16.6 |

TABLE 22-4
the result in sensory evaluation

| # | Metallic aftertaste | bitterness | Sweet lingering |
|---|---|---|---|
| 2-1 | 2 | 0 | 37 s |
| 2-2 | 2 | 0 | 30 s |
| 2-3 | 2 | 0 | 26 s |
| 2-4 | 1 | 0 | 25 s |
| 2-5 | 1 | 0 | 18 s |
| 2-6 | 1 | 0 | 18 s |

The taste profile of RD can be improved by enzyme-transformed RU. As the ratio of ET-RU to RD increased, the metallic aftertaste is reduced although there is almost no bitterness in RD. The sweet lingering of RD can be shortened from about 37 seconds to about 20 seconds. Especially when the ratio of ET-RU to RD reached 20/80, the effect of decreased lingering was significant.

3. The improvement of enzyme-transformed RU to RM

TABLE 22-5
the weight of RM and ET-RU

| | | | Weight of ET-RU |
|---|---|---|---|
| # | The ratio of RM to ET-RU | Weight of RM (mg) | (mg) |
| 3-1 | 100/0 | 50 | 0 |
| 3-2 | 95/5 | 50 | 2.6 |
| 3-3 | 90/10 | 50 | 5.6 |
| 3-4 | 85/15 | 50 | 8.8 |
| 3-5 | 80/20 | 50 | 12.5 |
| 3-6 | 75/25 | 50 | 16.6 |

TABLE 22-6
the result in sensory evaluation

| # | Metallic aftertaste | bitterness | Sweet lingering |
|---|---|---|---|
| 3-1 | 1 | 0 | 25 s |
| 3-2 | 1 | 0 | 24 s |
| 3-3 | 1 | 0 | 20 s |
| 3-4 | 1 | 0 | 21 s |
| 3-5 | 2 | 0 | 18 s |
| 3-6 | 2 | 0 | 17 s |

The taste profile of RM can be improved by enzyme-transformed RU. Although there is almost no bitterness and metallic aftertaste in RM, the sweet lingering of RM is longer. The sweet lingering of RM can be shortened from about 25 seconds to about 20 seconds. Especially when the ratio of ET-RU to RM reached 20/80, the effect of decreased lingering was significant.

### Example 23

The experiments performed were designed to investigate the effect of Reb-B and rubusoside on the taste profile of NHDC.

Materials

Neohesperidine dihydrochalcone (NHDC), ≥96%, Lot # MKBT9446V, Sigma Aldrich, Rebaudioside-B, Lot # RB100122, EPC Lab, Rubusoside, Lot # EPC-182-80-01, Sweet Green Fields

Sample Preparation

A solution of 10 ppm NHDC was prepared in water. Increasing amounts of Reb-B (1-5 pmm) were added to the 10 ppm NHDC solution.

A solution of 10 ppm NHDC was prepared in water. Increasing amounts of Rubusoside were (1-5 ppm) added to the 10 ppm NHDC solution.

Sensory Evaluation

Before tasting the tasters discussed the upcoming series of samples to be tasted to arrive at a suitable description. Four trained tasters blind taste tested independently all samples of a series. The taste testers were allowed to re-taste and made notes for the sensory attributes perceived by the tester including the relative intensity.

The attributes noted were discussed amongst the testers to arrive at a suitable description agreeable to all. In the case that more than 1 taster disagreed with the description, the tasting was repeated.

Results

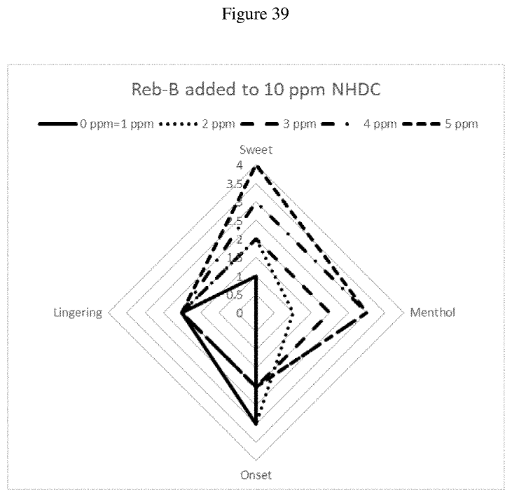

The effects of added Reb-B on a standard NHDC solution are shown in Table 23-1 and FIG. 39.

TABLE 23-1
Effect of Reb-B on a 10 ppm solution of NHDC

| Reb-B | Sweet | Menthol | Onset* | Lingering |
|---|---|---|---|---|
| 0 ppm | 1 | 0 | 3 | 2 |
| 1 ppm | 1 | 0 | 3 | 2 |
| 2 ppm | 2 | 1 | 2 | 3 |
| 3 ppm | 3 | 1 | 2 | 3 |
| 4 ppm | 4 | 2 | 1 | 4 |
| 5 ppm | 5 | 3 | 1 | 4 |

*Onset of sweetness (0-quick to 5-very slow)

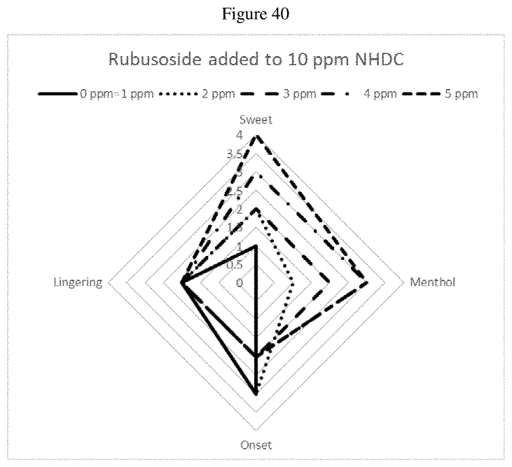

Table 23-2 and FIG. 40 demonstrate the effects of added rubusoside on a standard NHDC solution.

TABLE 23-2
Effect of rubusoside on a 10 ppm solution of NHDC

| Rubusoside | Sweet | Menthol | Onset* | Lingering |
|---|---|---|---|---|
| 0 ppm | 1 | 0 | 3 | 2 |
| 1 ppm | 1 | 0 | 3 | 2 |
| 2 ppm | 2 | 1 | 3 | 2 |
| 3 ppm | 2 | 2 | 2 | 2 |
| 4 ppm | 3 | 3 | 2 | 2 |
| 5 ppm | 4 | 3 | 2 | 2 |

*Onset of sweetness (0-quick to 5-very slow)

The results showed that small molecular stevia glycosides, such as Reb B or rubusoside, can influence the taste profile of NHDC or its related products significantly. Depending on type of low molecular weight steviol glycoside(s) added, it can create the surprisingly new flavor(s). For example, Reb-B and Rubusoside influence the sweetness profile of NHDC, and most surprisingly a menthol-type taste appears to be dose-dependent with the addition of Reb-B or Rubusoside. An embodiment of composition comprises low molecular weight steviol glycosides and NHDC or other naringine type of products, where the ratio between low molecular weight stevia glycosides with NHDC is from 1:99 to 99:1. Embodiments include methods to use compositions comprising low molecular weight stevia glycosides (LMWSGs) such as Reb B, and or rubusoside, to improve the taste of NDHC or naringine related products. Embodiments include compositions comprising low molecular weight stevia glycosides (LMWSGs) and NHDC in food and beverages, where the concentration of low molecular weight stevia glycosides is from 0.1 ppm to 1,000 ppm and concentration of NHDC is from 0.1 ppm to 30 ppm.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A composition comprising one or more of rubusoside (RU), steviolmonoside (STM) or steviolbioside (STB) with rebaudioside A (RA) and rebaudioside B (RB), wherein the composition has a reduced sweet lingering aspect that is less than that of the RA/RB combination without the RU, STM or STB present.

2. The composition of claim 1, wherein the combination of RA to RB is from about 10:90 to about 90:10 by weight.

3. The composition of claim 2, wherein the combination of RA to RB is about 75:15 by weight.

4. The composition of claim 1, wherein the ratio of the RU, STM or STB is about 1:4 by weight relative to the weight of the RA/RB combination.

5. The composition of claim 1, wherein the combination of RA to RB is about 75:15 by weight and the ratio of the RU, STM or STB is about 1:4 by weight of the weight of the RA/RB combination.

6. A composition comprising one or more of rebaudioside A (RA), rebaudioside B (RB) or rebaudioside D (RD) with rubusoside (RU), wherein a sweet lingering aspect of the composition is decreased in comparison to that of the RA, RB or RD without the RU present.

7. The composition of claim 6, wherein the ratio of RA, RB, or RD to RU is about 20:1 to about 5:1.

8. The composition of claim 7, wherein the ratio of RA, RB or RD to RU is about 9:1 by weight.

9. A composition comprising thaumatin and one or more of rubusoside (RU) or steviolbioside (STB), wherein a sweet lingering aspect of the composition is decreased in comparison to that of the thaumatin without the RU or STB present.

10. The composition of claim 9, wherein the ratio of thaumatin to the RU or STB is about 1:10 to about 1:1.

11. The composition of claim 10, wherein the ratio of the thaumatin to the RU or STB is about 5:9.

12. A composition comprising rebaudioside D (RD) in combination with one or more of steviolbioside (STB), dulcoside B (DB), steviolmonoside (STM) or rubusoside (RU), wherein a sweet lingering aspect of the composition is decreased in comparison to that of the RD without the STB, DB, STM or RU present.

13. The composition of claim 12, wherein the ratio of the RD to STB, DB, STM or RU is from about 20:1 to about 5:1.

14. The composition of claim 13, wherein the ratio of the RD to STB, DB, STM or RU is from about 9:1 to about 7.5:2.5.

15. A composition comprising rebaudioside M (RM) in combination with one or more of steviolbioside (STB), dulcoside B (DB), steviolmonoside (STM) or rubusoside (RU), wherein a sweet lingering aspect of the composition is decreased in comparison to that of the RM without the STB, DB, STM or RU present.

16. The composition or claim 15, wherein the ratio of the RM to STB, DB, STM or RU is from about 20:1 to about 5:1.

17. The composition of claim 16, wherein the ratio of the RM to STB, DB, STM or RU is from about 9:1 to about 7.5:2.5.

18. A composition comprising rebaudioside A (RA) in combination with one or more of steviolbioside (STB), dulcoside B (DB), steviolmonoside (STM) or rubusoside (RU), wherein a sweet lingering aspect of the composition is decreased in comparison to that of the RA without the STB, DB, STM or RU present.

19. The composition of claim 18, wherein the ratio of the RA to STB, DB, STM or RU is from about 20:1 to about 5:1.

20. The composition of claim 19, wherein the ratio of the RA to STB, DB, STM or RU is from about 9:1 to about 7.5:2.5.

21. A composition comprising neohesperidine dihydrochalcone (NHDC) in combination with a low molecular weight steviol glycoside (LMWSG) having a molecular weight of less than or equal to about 965 daltons, wherein a menthol aspect of the composition is increased in comparison to that of the NHDC without a LMWSG present.

22. The composition of claim 21, wherein the ratio of NHDC to the LMWSG is from about 10:1 to about 1:1.

23. The composition of claim 22, wherein the ratio of NHDC to the LMWSG is from about 5:1 to about 2:1.

24. The composition of claim 21, wherein the LMWSG comprises rebaudioside B (RB) or rubusoside (RU).

25. The composition of claim 24, wherein the ratio of the NHDC to RB or RU is from about 10:1 to about 1:1.

26. The composition of claim 25, wherein the ratio of the NHDC to RB or RU is from about 5:1 to about 2:1.

27. A composition comprising two or more steviol glycosides (SGs) having a low molecular weight (LMWSG) of equal to or less than 965 daltons.

28. The composition of claim 27, wherein the SGs comprise two or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A, Reb A2 (SG-7), Reb E and Reb H1.

29. A composition comprising rebaudioside A from about 50 to about 70% by weight of the total steviol glycosides in the composition and one or more steviol glycosides having a low molecular weight of less than or equal to 965 daltons present in greater than about 10 to about 30% by weight of the total steviol glycosides in the composition.

30. The composition of claim 29, wherein the low molecular weight steviol glycosides comprise one or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb B, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A2 (SG-7), Reb E or Reb H1.

31. A composition comprising rebaudioside A from about 50 to about 70% by weight and from about 5 to about 15% by weight rebaudioside B of the total steviol glycosides in the composition and one or more steviol glycosides having a molecular weight of less than or equal to 965 daltons present in greater than 10 to about 30% by weight of the total steviol glycosides in the composition.

32. The composition of claim 31, wherein the SGs comprise one or more of related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D, iso-Reb B, iso-stevioside, Reb G, Reb-KA, SG-13, stevioside, stevioside B (SG-15), Reb F, Reb R, SG-Unk2, SG-Unk3, Reb F3, (SG-11), Reb F2 (SG-14), Reb C, Reb C2/Reb S, stevioside E (SG-9), stevioside E2, SG-10, Reb L1, SG-2 Reb A3, (SG-8), iso-Reb A, Reb A2 (SG-7), Reb E and Reb H1.

33. A composition comprising a sweet tea extract, a stevia extract, a swingle extract, a glycosylated sweet tea extract, a glycosylated stevia extract, a glycosylated swingle extract, a glycosylated sweet tea glycoside, a glycosylated steviol glycoside, a glycosylated mogroside or mixtures thereof and one or more steviol glycoside(s) having a molecular weight of less than or equal to 965 daltons present in greater than about 10 to about 50% by weight of the total steviol glycosides in the composition.

34. The composition of claim 33, wherein the stevia extract is one or more of rebaudioside A, rebaudioside B, rebaudioside D, rebaudioside E, rebaudioside M, rebaudioside O, or mixtures thereof.

35. The composition of claim 33, wherein the swingle extract comprises one or more mogroside extract components.

36. The composition of claim 35, wherein the mogroside extract component is one or more of mogroside V, mogroside IV, siamenoside I, 11-oxomogroside V or mixtures thereof.

37. The composition of claim 33, wherein the glycosylated stevia extract comprises glycosylation products of steviol, stevioside, steviolbioside, rebaudioside A, rebaudioside B, rebaudioside C, rebaudioside D, rebaudioside E, rebaudioside F, rebaudioside M, rebaudioside O, rebaudioside H, rebaudioside I, rebaudioside L, rebaudioside N, rebaudioside K, rebaudioside J, rubusoside, dulcoside A or mixtures thereof.

38. The composition of claim 33, wherein the glycosylated steviol glycoside comprises glycosylation products of steviol, stevioside, steviolbioside, rebaudioside A, rebaudioside B, rebaudioside C, rebaudioside D, rebaudioside E, rebaudioside F, rebaudioside M, rebaudioside O, rebaudioside H, rebaudioside I, rebaudioside L, rebaudioside N, rebaudioside K, rebaudioside J, rubusoside, dulcoside A or mixtures thereof.

39. The composition of claim 33, wherein the glycosylated swingle extract comprises a glycosylated mogroside II, a glycosylated mogroside III, a glycosylated mogroside IV, a glycosylated mogroside V, a glycosylated siamenoside I or a glycosylated 11-oxomogroside V or mixtures thereof.

40. The composition of claim 33, wherein the glycosylated mogroside comprises a glycosylated mogroside II, a glycosylated mogroside III, a glycosylated mogroside IV, a glycosylated mogroside V, a glycosylated siamenoside I or a glycosylated 11-oxomogroside V or mixtures thereof.

41. A composition comprising rebaudioside D, rebaudioside M, a mixture of rebaudioside D and rebaudioside M or a mixture of rebaudioside A, rebaudioside D and rebaudioside M and a low molecular weight steviol glycoside (LMWSG) having a molecular weight of equal to or less than 965 daltons.

42. The composition of claim 41, wherein the LMWSG comprises related SvGn #1, steviol-monoside, steviol-monoside A, SG-4, dulcoside A1, iso-steviolbioside, Reb-G1, rubusoside, steviolbioside, related SvGn #3, Reb-F1, Reb-R1, stevioside F (SG-1), SG-Unk1, dulcoside A, dulcoside B (JECFA C), SG-3, stevioside D or mixtures thereof.

43. A flavoring or a sweetener composition comprising steviol glycosides wherein low molecular weight steviol glycosides (LMWSGs) having a molecular weight of less than or equal to 787 are present in solution at a range of 1 ppm to about 5000 ppm.

44. A flavoring or a sweetener composition comprising steviol glycosides consisting of at least two low molecular weight steviol glycoside(s) (LMWSGs).

45. A stevia composition comprising two or more steviol glycosides selected from steviol monoside, steviolbioside, rubusoside, dulcoside.

46. A method to reduce the lingering of sweetness in a sweetened composition comprising the step:
providing a sweetener composition having a sweetness lingering time period of X;
adding a low molecular weight steviol glycoside (LMWSG) to the sweetener composition, wherein the sweetness lingering time period of X is reduced by at least 30 percent.

47. The method of claim 46, wherein the LMWSG is selected from the group consisting of steviolmonoside, steviolbioside, dulcoside, rubusoside or mixtures thereof.

48. The method of claim 47, wherein the sweetener composition further comprises thaumatin or neohesperidine dihydrochalcone (NHDC).

49. The composition of claim 27, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

50. The composition of claim 29, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

51. The composition of claim 31, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

52. The composition of claim 33, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

53. The composition of claim 41, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

54. The composition of claim 43, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

55. The composition of claim 44, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

56. The composition of claim 45, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).

57. The method of claim 46, further comprising one or more monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, and cyclocarioside I, sugar alcohols, such as erythritol, sucralose, acesulfame acid and salts thereof, such as acesulfame-K and potassium acesulfame; N-(L-α-aspartyl)-L-phenylalanine methylester (Aspartame), N—[N-[3-(3-hydroxy-4-methoxyphenyl) propyl]-α-aspartyl]-L-phenylalanine (Advantame), N—[N-[3-(3-hydroxy-4-methoxyphenyl) propyl]-α-aspartyl]-L-phenylalanine 1-methyl ester (ANS9801), alitame, saccharin and salts thereof, neohesperidin dihydrochalcone, cyclamate, cyclamic acid and salts thereof, neotame, trehalose, raffinose, cellobiose, tagatose, allulose, inulin, or neohesperidine dihydrochalcone (NHDC).
